(19) United States
(12) Patent Application Publication
(10) Pub. No.: US 2005/0009845 A1
(43) Pub. Date: Jan. 13, 2005

(54) Thienopyrimidine compounds as protein tyrosine kinase inhibitors

(76) Inventors: Thomas R. Caferro, Durham, NC (US); Stanley Dawes Chamberlain, Durham, NC (US); Kelly Horne Donaldson, Durham, NC (US); Philip Anthony Harris, Durham, NC (US); Michael David Gaul, Yardley, PA (US); David Edward Uehling, Durham, NC (US); Dana Edward Vanderwall, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/499,247

(22) PCT Filed: Dec. 13, 2002
(86) PCT No.: PCT/US02/39872

(30) Foreign Application Priority Data
Dec. 19, 2001 (US) 60342207

(51) Int. Cl. A61K 31/519; C07D 498/02

(52) U.S. Cl. 514/260.1; 544/279

(57) ABSTRACT

The present invention relates to thienopyrmidine compounds of formula (I) (one of A¹ and A² is S and the other is CH), salts thereof, as well as use and preparation of the same. These compounds are inhibitors of various protein tyrosine kinases (PTKs) of the ErbB family and consequently are useful in the treatment of disorders mediated by aberrant activity of such kinases.

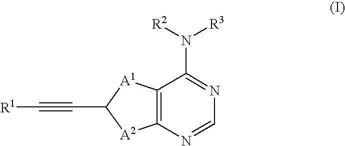

### FIELD OF THE INVENTION

The present invention relates to thienopyrimidine compounds, salts thereof, as well as use and preparation of the same. These compounds are inhibitors of various protein tyrosine kinases (PTKs) of the ErbB family and consequently are useful in the treatment of disorders mediated by aberrant activity of such kinases.

### BACKGROUND OF THE INVENTION

PTKs catalyze the phosphorylation of specific tyrosyl residues in various proteins involved in the regulation of cell growth and differentiation. (A. F. Wilks, Progress in Growth Factor Research, 1990, 2, 97-111; S. A. Courtneidge, Dev. Supp.l, 1993, 57-64; J. A. Cooper, Semin. Cell Biol., 1994, 5(6), 377-387; R. F. Paulson, Semin. Immunol., 1995, 7(4), 267-277; A. C. Chan. Curr. Opin. Immunol., 1996, 8(3), 394-401). Inappropriate or uncontrolled activation of many PTKs, i.e. aberrant PTK activity, for example by over-expression or mutation, has been shown to result in uncontrolled cell growth.

Aberrant protein tyrosine kinase (PTK) activity has been implicated in a variety of disorders including psoriasis, rheumatoid arthritis, bronchitis, as well as cancer. Development of effective treatments for such disorders is a constant and ongoing enterprise in the medical field. The ErbB family of PTKs, which includes c-ErbB-2, EGFR, and ErbB-4, is one group of PTKs that has attracted interest as a therapeutic target. Currently, of special interest, is the role of ErbB family PTKs in hyperproliferative disorders, particularly human malignancies. Elevated EGFR activity has, for example, been implicated in non-small cell lung, bladder, and head and neck cancers. Furthermore, increased c-ErbB-2 activity has been implicated in breast, ovarian, gastric and pancreatic cancers. Consequently, inhibition of ErbB family PTKs should provide a treatment for disorders characterized by aberrant ErbB family PTK activity. The biological role of ErbB family PTKs and their implication in various disease states is discussed, for instance in U.S. Pat. No. 5,773,476; International Patent Application WO 99/35146; M. C. Hung et al, Seminars in Oncology, 26: 4, Suppl. 12 (August) 1999, 51-59; Ullrich et al, Cell, 61: 203-212, Apr. 20, 1990; Modjtahedi et al, Int'l. J. of Oncology, 13: 335-342, 1998; and J. R. Woodbum, Pharmacol. Ther., 82: 2-3, 241-250, 1999.

International Patent Application PCT/EP99/00048 filed Jan. 8, 1999, and published as WO 99/35146 on Jul. 15, 1999, discusses PTKs including ErbB family PTKs. This published application discloses bicyclic heteroaromatic compounds, including N-{3-Chloro-4-[(3-fluorobenzyl)oxy]phenyl}-6-[5-({[2-(methanesulphonyl)ethyl]amino}methyl)-2-furyl]-4-quinazolinamine; (4-(3-Fluoro-benzyloxy)-3-chloro phenyl)-(6-(2-((2-methanesulphonyl-ethylaminoymethyl)-thiazol-4-yl)quinazolin-4-yl)-amine; and (4-(3-Fluoro-benzyloxy)-3-bromophenyl)-(6-(5-((2-methane sulphonyl-ethylamino)-methyl)-furan-2-yl)quinazolin-4-yl)-amine as well as hydrochloride salts thereof. These compounds show inhibition activity against ErbB family PTKs.

International Patent Application PCT/IB98/01691 filed Oct. 22, 1998, and published as WO 99/24440 on May 20, 1999, discusses the use of certain thienopyrimidine and thienopyrimidine derivatives and their use in treating hyperproliferative disorders.

International Patent Application PCT/GB00/01006 filed Mar. 17, 2000, and published as WO 00/56738 on Sep. 28, 2000, discloses certain pyrimidine and pyrimidine derivatives useful as inhibitors of cytokine mediated disease.

U.S. Pat. No. 6,174,889 B1 discloses certain bicyclic heteroaromatic compounds useful as protein tyrosine kinase inhibitors.

U.S. Pat. No. 5,747,486 discloses certain thienopyrimidine and thienopyrimidine derivatives useful as anti-inflammatory or bone resorption inhibiting agents.

U.S. Pat. No. 6,130,223 discloses certain thienopyrimidine compounds with phosphodiesterase V activity.

U.S. Pat. No. 6,133,271 discloses certain thienopyrimidine compounds for inducing or promoting apoptosis and for arresting uncontrolled neoplastic cell proliferation.

### SUMMARY OF THE INVENTION

The present invention provides compounds suitable for the treatment of disorders mediated by protein kinase activity, in particular hyperproliferative disorders.

In addition to the treatment of hyperproliferative disorders, the present invention contemplates that other disorders mediated by protein kinase activity may be treated by inhibition, including preferential inhibition, of the appropriate protein kinase activity.

Broad spectrum inhibition of protein kinase activity may not always provide optimal treatment of certain diseases, tumors for example, and could in certain cases even be detrimental to subjects since protein kinases provide an essential function in the regulation of normal cell growth.

It is another object of the present invention to provide compounds that preferentially inhibit protein tyrosine kinases, such as EGFR, c-ErbB-2, c-met, tie-2, PDGFr, s-src, Ick, Zap 70, and fyn. There is also perceived to be a benefit in the preferential inhibition involving small groups of protein tyrosine kinases, for example c-ErbB-2 and c-ErbB-4 or c-ErbB-4 and EGF-R.

A further object of the present invention is to provide compounds useful in the treatment of protein tyrosine kinase related diseases that minimize undesirable side effects in the recipient.

The present invention relates to heterocyclic compounds that may be used to treat disorders mediated by protein tyrosine kinases arid have anti-cancer properties. More particularly, the compounds of the present invention are potent inhibitors of protein tyrosine kinases such as EGFR, c-ErbB-2, c-ErbB-4, c-met, tie-2, PDGFr, c-src, Ick, Zap70, and fyn, thereby allowing clinical management of particular diseased tissues.

The present invention contemplates, in particular, the treatment of human malignancies, for example breast, non-small cell lung, ovary, stomach, and pancreatic tumors, especially those mediated by EGFR or ErbB-2, using the compounds of the present invention. For example, the invention includes compounds that are highly active against the c-ErbB-2 protein tyrosine kinase often in preference to the EGF receptor kinase, thereby allowing treatment of c-ErbB-2 mediated tumors. However, the invention also includes compounds that are highly active against both the c-ErbB-2 and EGFR receptor kinases, thereby allowing treatment of a broad range of tumors.

More particularly, the invention contemplates that disorders mediated by protein tyrosine kinase activity may be treated effectively by inhibition of the appropriate protein tyrosine kinase activity in a relatively selective manner, thereby minimizing potential side-effects.

### DETAILED DESCRIPTION OF THE INVENTION

Accordingly, the present invention provides compounds of formula (I), or salts thereof 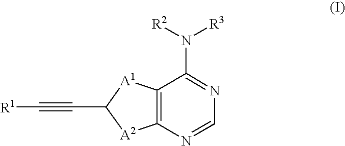
wherein:
one of A¹ and A² is S and the other is CH;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R⁶)—C(S)—N(R⁶)(R⁷), —N(R⁶)—C(O)—OR⁷, —N(R⁶)—C(O) —(CH₂)ₙ—R⁷, —N(R⁶)—SO₂R⁶, —(CH₂)ₙNR⁶R⁷, —(CH₂)ₙOR⁷, —(CH₂)ₙSR⁸, —(CH₂)ₙS(O)R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, —(CH₂)ₙS(O)₂R⁸ , —(CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
one of A¹ and A² is S and the other is CH;
R¹ is —(CR¹¹R¹¹)ₙ—R⁵;
R² is H;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, (CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₆cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
one of A¹ and A² is S and the other is CH;
R¹ is —(CR¹¹R¹¹)ₙ—R⁵;
R² is H;
R³ is aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
one of A¹ and A² is S and the other is CH;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R⁶)—C(S)—N(R⁶)(R⁷), —N(R⁶)—C(O)—OR⁷, —N(R⁶)—C(O)—(CH₂)ₙ—R⁷, —N(R⁶)—SO₂R⁶, —(CH₂)ₙNR⁶R⁷, —(CH₂)ₙOR⁷, —(CH₂)ₙSR⁸, —(CH₂)ₙS(O)R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, —(CH₂)ₙS(OR⁸, —(CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is CH and A² is S;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R⁶)—C(S)—N(R⁶)(R⁷), —N(R⁶)—C(O)—OR⁷, —N(R⁶)—C(O)—(CH₂)ₙ—R⁷, —N(R⁶)—SO₂R⁶, —(CH₂)ₙNR⁶R⁷, —(CH₂)ₙOR⁷, —(CH₂)ₙSR⁸, —(CH₂)ₙS(O)R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, —(CH₂)ₙS(O)₂R⁸, —(CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰ or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R₁₀, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R₁₀;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵;
R² is H;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is CH and A² is S;
R¹ is —(CR¹¹R¹¹)ₙ—R⁵;
R² is H;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR₈, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵;
R² is H;
R³ is aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ CH and A² is S;
R¹ is —(CR¹¹R¹¹)ₙ—R⁵;
R² is H;
R³ is aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and (CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is CH and A² is S;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R₁₀;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵;
R² is H;
R³ is aryl optionally substituted with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR9R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵;
R² is H;
R³ is aryl optionally substituted in the para position with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ is S and A² is CH;
R¹ is —(CR¹¹R¹¹)ₙ—R⁵, n=0-6;
R² is H;
R³ is aryl optionally substituted in the para position with —(CH₂)ₙOR⁴, and in the meta position with halogen, —CN, C₁₋₆alkyl, or alkynyl;
R⁴ is —(CH₂)ₙaryl in which aryl is optionally substituted with halo;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring, n=0-6;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n=0-6.

In another aspect of the invention are compounds of formula (I), wherein:
A¹ CH and A² is S;
R¹ is —(CR¹¹R¹¹)ₙ—R⁵;
R² is H;
R³ is aryl optionally substituted in the para position with —(CH₂)ₙOR⁴ and in the meta position with halogen, —CN, C₁₋₆alkyl, or alkynyl;
R⁴ is —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is —N(R⁶)—C(O)—N(R⁶)(R⁷), —N(R⁶)—C(O)—(CH₂)ₙ—R⁷, or —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ CH and A² is S;
R¹ is —(CR¹¹R¹¹)ₙ—R⁵;
R² is H;
R³ is aryl substituted in the para position with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵ is —N(R⁶)—C(O)—N(R⁶)(R⁷), —N(R⁶)—C(O)—(CH₂)ₙ—R⁷, or —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

In another aspect of the invention are compounds of formula (I), wherein:
A¹ CH and A² is S;
R¹ is —(CR¹¹R¹¹)ₙ—R⁵;
R² is H;
R³ is aryl substituted in the para position with —(CH₂)ₙOR⁴ and in the meta position with halogen, —CN, C₁₋₆alkyl, or alkynyl;
R⁴ is —(CH₂)ₙaryl substituted with halo;
R⁵ is —N(R⁶)—C(O)—N(R⁶)(R⁷), —N(R⁶)—C(O)—(CH₂)ₙ—R⁷, or —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n=0-6.

The present invention also provides the following compounds:
N-(2-benzyl-1H-benzimidazol-5-yl)-6-ethynylthieno[3,2-d]pyrimidin-4-amine;
N-(2-benzyl-1H-benzimidazol-5-yl)-6-(1H-pyrazol-4-ylethynyl)thieno[3,2-d]pyrimidin-4-amine;
N-{3-chloro-4-[(3-fluorobenzyl)oxy]phenyl}-6-ethynylthieno[3,2-d]pyrimidin-4-amine;
N-(2-benzyl-1H-benzimidazol-5-yl)-6-[3-(1,1-dioxidothiomorpholin-4-yl)prop-1-ynyl]thieno[3,2-d]pyrimidin-4-amine;
N-{3-chloro-4-[(3-fluorobenzyl)oxy]phenyl}-6-[3-(1,1-dioxidothiomorpholin-4-yl)prop-1-ynyl]thieno[3,2-d]pyrimidin-4-amine hydrochloride;
N-(1-benzyl-1H-indazol-5-yl)-6-[3-(1,1-dioxidothiomorpholin-4-yl)prop-1-ynyl]thieno[3,2-d]pyrimidin-4-amine hydrochloride;
N-(1-benzyl-1H-indazol-5-yl)-6-ethynylthieno[2,3-d]pyrimidin-4-amine;
N-(2-benzyl-1H-benzimidazol-6-yl)-6-ethynylthieno[2,3-d]pyrimidin-4-amine;
N-{3-chloro-4-[(3-fluorobenzyl)oxy]phenyl}-6-ethynylthieno[2,3-d]pyrimidin-4-amine;
N-(1-benzyl-1H-indazol-5-yl)-6-(3-morpholin-4-ylprop-1-ynyl)thieno[3,2-d]pyrimidin-4-amine hydrochloride;
N-(2-benzyl-1H-benzimidazol-5-yl)-6-(3-morpholin-4-ylprop-1-ynyl)thieno[3,2-d]pyrimidin-4-amine;
N-{3-chloro-4-[(3-fluorobenzyl)oxy]phenyl}-6-(3-morpholin-4-ylprop-1-ynyl)thieno[3,2-d]pyrimidin-4-amine hydrochloride;
N-(1-benzyl-1H-benzimidazol-5-yl)-6-(3-morpholin-4-ylprop-1-ynyl)thieno[3,2-d]pyrimidin-4-amine hydrochloride;
N-{3-[4-({3-chloro-4-[(3-fluorobenzyl)oxy]phenyl}amino)thieno[3,2-d]pyrimidin-6-yl]prop-2-ynyl}urea hydrochloride;
N-(3-{4-[(1-benzyl-1H-indazol-5-yl)amino]thieno[3,2-d]pyrimidin-6-yl}prop-2-ynyl)urea hydrochloride;
N-(3-{4-[(2-benzyl-1,3-benzoxazol-6-yl)amino]thieno[3,2-d]pyrimidin-6-yl}prop-2-ynyl)urea hydrochloride;
N-(2-benzyl-1,3-benzoxazol-6-yl)-6-ethynylthieno[3,2-d]pyrimidin-4-amine hydrochloride;
N-(1-benzyl-1H-indol-5-yl)-6-ethynylthieno[3,2-d]pyrimidin-4-amine hydrochloride;
N-(2-benzyl-1-benzofuran-5-yl)-6-ethynylthieno[3,2-d]pyrimidin-4-amine hydrochloride;
N-(3-{4-[(2-benzyl-1H-benzimidazol-5-yl)amino]thieno[3,2-d]pyrimidin-6-yl}prop-2-ynyl)urea hydrochloride;
N-(2-benzyl-1,3-benzothiazol-6-yl)-6-ethynylthieno[3,2-d]pyrimidin-4-amine hydrochloride;
N-(2-benzyl-1,3-benzothiazol-5-yl)-6-ethynylthieno[3,2-d]pyrimidin-4-amine hydrochloride;
N-(4-benzylphenyl)-6-ethynylthieno[3,2-d]pyrimidin-4-amine;
6-ethynyl-N-[4-(1-naphthyloxy)phenyl]thieno[3,2-d]pyrimidin-4-amine hydrochloride;
b 6-ethynyl-N-[4-(3-methoxyphenoxy)phenyl]thieno[3,2-d]pyrimidin-4-amine hydrochloride;
6-ethynyl-N-[4-(4-methylphenoxy)phenyl]thieno[3,2-d]pyrimidin-4-amine hydrochloride;
6-ethynyl-N-[4-(4-methylphenoxy)phenyl]thieno[3,2-d]pyrimidin-4-amine;
N-{3-chloro-4-[(3-fluorobenzyl)oxy]phenyl}-6-[3-(dimethylamino)prop-1-ynyl]thieno[3,2-d]pyrimidin-4-amine hydrochloride;
N-{3-{[4-({3-chloro-4-[(3-fluorobenzyl)oxy]phenyl}amino)thieno[3,2-d]pyrimidin-6-yl]ethynyl}phenyl)acetamide hydrochloride;
N-[3-({4-[(1-benzyl-1H-indazol-5-yl)amino]thieno[3,2-d]pyrimidin-6-yl}ethynyl)phenyl]acetamide hydrochloride;
N-[3-({4-[(2-benzyl-1H-benzimidazol-5-yl)amino]thieno[3,2-d]pyrimidin-6-yl}ethynyl)phenyl]acetamide hydrochloride;
tert-butyl 3-[4-({3-chloro-4-[(3-fluorobenzyl)oxy]phenyl}amino)thieno[3,2-d]pyrimidin-6-yl]prop-2-ynylcarbamate hydrochloride;
tert-butyl 3-{4-[(1-benzyl-1H-indazol-5-yl)amino]thieno[3,2-d]pyrimidin-6-yl}prop-2-ynylcarbamate hydrochloride;
N-{3-[4-({3-chloro-4-[(3-fluorobenzyl)oxy]phenyl}amino)thieno[3,2-d]pyrimidin-6-yl]prop-2-ynyl}acetamide hydrochloride;
N-(3-{4-[(1-benzyl-1H-indazol-5-yl)amino]thieno[3,2-d]pyrimidin-6-yl}prop-2-ynyl)acetamide hydrochloride;
N-(3-{4-[(2-benzyl-1H-benzimidazol-5-yl)amino]thieno[3,2-d]pyrimidin-6-yl}prop-2-ynyl)acetamide hydrochloride;
6-ethynyl-N-(4-{[3-(trifluoromethyl)phenyl]thio}phenyl)thieno[3,2-d]pyrimidin-4-amine;
6-ethynyl-N-[2-(3-methoxybenzyl)-1H-benzimidazol-5-yl]thieno[3,2-d]pyrimidin-4-amine;
6-(3-aminoprop-1-ynyl)-N-(1-benzyl-1H-indazol-5-yl)thieno[3,2d]pyrimidin-4-amine;
6-(3-aminoprop-1-ynyl)-N-{3-chloro-4-[(3-fluorobenzyl)oxy]phenyl}thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-N′-methylurea;
N-{3-[4-({3-chloro-4-[(3-fluorobenzyl)oxy]phenyl}amino)thieno[3,2-d]pyrimidin-6-yl]prop-2-ynyl}-N′-cyclopentylurea;
N-{3-[4-({3-chloro-4-[(3-fluorobenzyl)oxy]phenyl}amino)thieno[3,2-d]pyrimidin-6-yl]prop-2-ynyl}-4-methylbenzenesulfonamide;
N-{3-[4-({3-chloro-4-[(3-fluorobenzyl)oxy]phenyl}amino)thieno[3,2-d]pyrimidin-6-yl]prop-2-ynyl}-N′-phenylurea;
N-{3-[4-({3-chloro-4-[(3-fluorobenzyl)oxy]phenyl}amino)thieno[3,2-d]pyrimidin-6-yl]prop-2-ynyl}-2-(4-methylpiperazin-1-yl)acetamide;
N-{3-[4-({3-chloro-4-[(3-fluorobenzyl)oxy]phenyl}amino)thieno[3,2-d]pyrimidin-6-yl]prop-2-ynyl}-4-(morpholin-4-ylmethyl)benzamide;
N-{3-[4-({3-chloro-4-[(3-fluorobenzyl)oxy]phenyl}amino)thieno[3,2-d]pyrimidin-6-yl]prop-2-ynyl}-2-(1-methyl-1H-imidazol-4-yl)acetamide;
N-{3-chloro-4-[(3-fluorobenzyl)oxy]phenyl}-6-(3-{[2-(methylsulfonyl)ethyl]amino}prop-1-ynyl)thieno[3,2-d]pyrimidin-4-amine hydrochloride;
N-{3-[4-({3-chloro-4-[(3-fluorobenzyl)oxy]phenyl}amino)thieno[3,2-d]pyrimidin-6-yl]prop-2-ynyl}-4-[(4-methylpiperazin-1-yl)methyl]benzamide;
N-{3-[4-({3-chloro-4-[(3-fluorobenzyl)oxy]phenyl}amino)thieno[3,2-d]pyrimidin-6-yl]prop-2-ynyl}-4-[(dimethylamino)methyl]benzamide;
N-{3-[4-({3-chloro-4-[(3-fluorobenzyl)oxy]phenyl}amino)thieno[3,2-d]pyrimidin-6-yl]prop-2-ynyl}-4-(1H-imidazol-1-ylmethyl)benzamide;
N˜1˜-{3-[4-({3-chloro-4-[(3-fluorobenzyl)oxy]phenyl}amino)thieno[3,2-d]pyrimidin-6-yl]prop-2-ynyl}-N˜2˜,N˜2˜-dimethylglycinamide;
N-{3-[4-({3-chloro-4-[(3-fluorobenzyl)oxy]phenyl}amino)thieno[3,2-d]pyrimidin-6-yl]prop-2-ynyl}-2-pyridin-3-ylacetamide;
N-{3-[4-({3-chloro-4-[(3-fluorobenzyl)oxy]phenyl}amino)thieno[3,2-d]pyrimidin-6-yl]prop-2-ynyl}-2-pyridin-4-ylacetamide;
N-[({3-[4-({3-chloro-4-[(3-fluorobenzyl)oxy]phenyl}amino)thieno[3,2-d]pyrimidin-6-yl]prop-2-ynyl}amino)carbonyl]-4-methylbenzenesulfonamide;
N-(3-{4-[(3-chloro-4-fluorophenyl)amino]thieno[3,2-d]pyrimidin-6-yl}prop-2-ynyl)urea hydrochloride;
N-{3-[4-({3-chloro-4-[(3-fluorobenzyl)oxy]phenyl}amino)thieno[3,2-d]pyrimidin-6-yl]prop-2-ynyl}-4-methylpiperazine-1-carboxamide;
N′-{3-[4-({3-chloro-4-[(3-fluorobenzyl)oxy]phenyl}amino)thieno[3,2-d]pyrimidin-6-yl]prop-2-ynyl}-N,N-dimethylurea;
N-{3-[4-({3-chloro-4-[(3-fluorobenzyl)oxy]phenyl}amino)thieno[3,2-d]pyrimidin-6-yl]prop-2-ynyl}-4-(methylsulfonyl)benzenesulfonamide;
N˜1˜-{3-[4-({3-chloro-4-[(3-fluorobenzyl)oxy]phenyl}amino)thieno[3,2-d]pyrimidin-6-yl]prop-2-ynyl}N˜4˜-phenylsuccinamide;
N-{3-[4-({3-chloro-4-[(3-fluorobenzyl)oxy]phenyl}amino)thieno[3,2-d]pyrimidin-6-yl]prop-2-ynyl}-5-nitro-1H-pyrrole-3-carboxamide;
N-{3-[4-({3-chloro-4-[(3-fluorobenzyl)oxy]phenyl}amino)thieno[3,2-d]pyrimidin-6-yl]prop-2-ynyl}-2-pyridin-4-yl-1,3-thiazole-4-carboxamide;
N-{3-[4-({3-chloro-4-[(3-fluorobenzyl)oxy]phenyl}amino)thieno[3,2-d]pyrimidin-6-yl]prop-2-ynyl}-1,3-benzothiazole-6-carboxamide;
N-{3-[4-({3-chloro-4-[(3-fluorobenzyl)oxy]phenyl}amino)thieno[3,2-d]pyrimidin-6-yl]prop-2-ynyl}-2,2,3,3-tetramethylcyclopropanecarboxamide;
N-{3-[4-({3-chloro-4-[(3-fluorobenzyl)oxy]phenyl}amino)thieno[3,2-d]pyrimidin-6-yl]prop-2-ynyl}-3-(4-fluorophenyl)propanamide;
N-{3-[4-({3-chloro-4-[(3-fluorobenzyl)oxy]phenyl}amino)thieno[3,2-d]pyrimidin-6-yl]prop-2-ynyl}-2-(methylsulfonyl)acetamide;
N-{3-[4-({3-chloro-4-[(3-fluorobenzyl)oxy]phenyl}amino)thieno[3,2-d]pyrimidin-6-yl]prop-2-ynyl}-2-thien-3-ylacetamide;
2,6-dichloro-N-{3-[4-({3-chloro-4-[(3-fluorobenzyl)oxy]phenyl}amino)thieno[3,2-d]pyrimidin-6-yl]prop-2-ynyl}benzamide;
N-{3-[4-({3-chloro-4-[(3-fluorobenzyl)oxy]phenyl}amino)thieno[3,2-d]pyrimidin-6-yl]prop-2-ynyl}-1H-indole-5-carboxamide;
N-{3-[4-({3-chloro-4-[(3-fluorobenzyl)oxy]phenyl}amino)thieno[3,2-d]pyrimidin-6-yl]prop-2-ynyl}-2-tetrahydro-2H-pyran-4-ylacetamide;
4-(benzyloxy)-N-{3-[4-({3-chloro-4-[(3-fluorobenzyl)oxy]phenyl}amino)thieno[3,2-d]pyrimidin-6-yl]prop-2-ynyl}benzamide;
N-{3-[4-({3-chloro-4-[(3-fluorobenzyl)oxy]phenyl}amino)thieno[3,2-d]pyrimidin-6-yl]prop-2-ynyl}-2-pyridin-2-ylacetamide;
N-{3-[4-({3-chloro-4-[(3-fluorobenzyl)oxy]phenyl}amino)thieno[3,2-d]pyrimidin-6-yl]prop-2-ynyl}-2-(2-furyl)acetamide;
N-{3-[4-({3-chloro-4-[(3-fluorobenzyl)oxy]phenyl}amino)thieno[3,2-d]pyrimidin-6-yl]prop-2-ynyl}isonicotinamide;
N-{3-[4-({3-chloro-4-[(3-fluorobenzyl)oxy]phenyl}amino)thieno[3,2-d]pyrimidin-6-yl]prop-2-ynyl}quinoline-2-carboxamide;
N′-{3-[4-({3-chloro-4-[(3-fluorobenzyl)oxy]phenyl}amino)thieno[3,2-d]pyrimidin-6-yl]prop-2-ynyl}-N,N-diisopropylurea;
N-{3-[4-({3-chloro-4-[(3-fluorobenzyl)oxy]phenyl}amino)thieno[3,2-d]pyrimidin-6-yl]prop-2-ynyl}-1-methyl-1H-pyrrole-2-carboxamide;
N-{3-[4-({3-chloro-4-[(3-fluorobenzyl)oxy]phenyl}amino)thieno[3,2-d]pyrimidin-6-yl]prop-2-ynyl}cinnoline-4-carboxamide;
2-(benzyloxy)-N-{3-[4-({3-chloro-4-[(3-fluorobenzyl)oxy]phenyl}amino)thieno[3,2-d]pyrimidin-6-yl]prop-2-ynyl}acetamide;
(2E)-N-{3-[4-({3-chloro-4-[(3-fluorobenzyl)oxy]phenyl}amino)thieno[3,2-d]pyrimidin-6-yl]prop-2-ynyl}-3-(4-methylphenyl)prop-2-enamide;
N-{3-[4-({3-chloro-4-[(3-fluorobenzyl)oxy]phenyl}amino)thieno[3,2-d]pyrimidin-6-yl]prop-2-ynyl)1H-indazole-3-carboxamide;
(4R)-N-{3-[4-({3-chloro-4-[(3-fluorobenzyl)oxy]phenyl}amino)thieno[3,2-d]pyrimidin-6-yl]prop-2-ynyl}-2-oxo-1,3-thiazolidine-4-carboxamide;
N-{3-[4-({3-chloro-4-[(3-fluorobenzyl)oxy]phenyl}amino)thieno[3,2-d]pyrimidin-6-yl]prop-2-ynyl}-4-(dimethylamino)butanamide;
N-{3-[4-({3-chloro-4-[(3-fluorobenzyl)oxy]phenyl}amino)thieno[3,2-d]pyrimidin-6-yl]prop-2-ynyl}-4-(1H-indol-3-yl)butanamide;
N-{3-[4-({3-chloro-4-[(3-fluorobenzyl)oxy]phenyl}amino)thieno[3,2-d]pyrimidin-6-yl]prop-2-ynyl}morpholine-4-carboxamide;
N-{3-[4-({3-chloro-4-[(3-fluorobenzyl)oxy]phenyl}amino)thieno[3,2-d]pyrimidin-6-yl]prop-2-ynyl}-N′-[2-(methylsulfonyl)ethyl]urea;
N-{3-[4-({3-chloro-4-[(3-fluorobenzyl)oxy]phenyl}amino)thieno[3,2-d]pyrimidin-6-yl]prop-2-ynyl}-N′-(2-morpholin-4-ylethyl)urea;
N-{3-[4-({3-chloro-4-[(3-fluorobenzyl)oxy]phenyl}amino)thieno[3,2-d]pyrimidin-6-yl]prop-2-ynyl}-N′-[2-(dimethylamino)ethyl]urea;
3-[4-({3-chloro-4-[(3-fluorobenzyl)oxy]phenyl}amino)thieno[3,2-d]pyrimidin-6-yl]prop-2-yn-1-ol hydrochloride;
N-{3-[4-({3-chloro-4-[(3-fluorobenzyl)oxy]phenyl}amino)thieno[3,2-d]pyrimidin-6-yl]prop-2-ynyl}-N′-(2-cyanoethyl)urea;
tert-butyl-3-[4-({3-chloro-4-[(3-fluorobenzyl)oxy]phenyl}amino)thieno[3,2-d]pyrimidin-6-yl]prop-2-ynyl(methyl)carbamate;
N-{3-chloro-4-[(3-fluorobenzyl)oxy]phenyl}-6-[3-(methylamino)prop-1-ynyl]thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-2-(1H-indol-3-yl)acetamide;
N-{3-[4-({3-chloro-4-[(3-fluorobenzyl)oxy]phenyl}amino)thieno[3,2-d]pyrimidin-6-yl]prop-2-ynyl}-2-(3,4-dichlorophenyl)acetamide;
N-{3-[4-({3-chloro-4-[(3-fluorobenzyl)oxy]phenyl}amino)thieno[3,2-d]pyrimidin-6-yl]prop-2-ynyl}-2-(4-iodophenyl)acetamide;
6-(3-aminoprop-1-ynyl)-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-[3-(dipropylamino)prop-1-ynyl]thieno[3,2-d]pyrimidin-4-amine;
N-{3-chloro-4-[(3-fluorobenzyl)oxy]phenyl}-6-[3-(diethylamino)prop-1-ynyl]thieno[3,2-d]pyrimidin-4-amine;
N-{3-chloro-4-[(3-fluorobenzyl)oxy]phenyl}-6-[3-(isopropylamino)prop-1-ynyl]thieno[3,2-d]pyrimidin-4-amine;
6-[3-(benzylamino)prop-1-ynyl]-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-(3-{methyl[2-(methylsulfonyl)ethyl]amino}prop-1-ynyl)thieno[3,2-d]pyrimidin-4-amine;
3-[4-({3-chloro-4-[(3-fluorobenzyl)oxy]phenyl}amino)thieno[3,2-d]pyrimidin-6-yl]prop-2-ynyl methanesulfonate;
3-({3-[4-({3-chloro-4-[(3-fluorobenzyl)oxy]phenyl}amino)thieno[3,2-d]pyrimidin-6-yl]prop-2-ynyl}amino)propanenitrile;
N-{3-chloro-4-[(3-fluorobenzyl)oxy]phenyl}-6-[3-(ethylamino)prop-1-ynyl]thieno[3,2-d]pyrimidin-4-amine;
N-{3-chloro-4-[(3-fluorobenzylpoxy]phenyl}-6-[3-(propylamino)prop-1-ynyl]thieno[3,2-d]pyrimidin-4-amine;
6-(3-amino-3-methylbut-1-ynyl)-N-{3-chloro-4-[(3-fluorobenzyl)oxy]phenyl}thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-2-(1H-indol-3-yl)acetamide;
N-{3-[4-({3-chloro-4-[(3-fluorobenzyl)oxy]phenyl}amino)thieno[3,2-d]pyrimidin-6-yl]prop-2-ynyl}-2-(4-iodophenyl)acetamide;
N-{3-[4-({3-chloro-4-[(3-fluorobenzyl)oxy]phenyl}amino)thieno[3,2-d]pyrimidin-6-yl]prop-2-ynyl}-2-(3,4-dichlorophenyl)acetamide;
N-{3-chloro-4-[(3-fluorobenzyl)oxy]phenyl}-6-[3-(diethylamino)prop-1-ynyl]thieno[3,2d]pyrimidin-4-amine;
6-(3-aminoprop-1-ynyl)-N-[4-(1-naphthyloxy)phenyl]thieno[3,2-d]pyrimidin-4-amine;
N-{3-chloro-4-[(3-fluorobenzyl)oxy]phenyl}-6-[3-(methylamino)prop-1-ynyl]thieno[3,2-d]pyrimidin-4-amine;
tert-butyl 3-[4-({3-chloro-4-[(3-fluorobenzyl)oxy]phenyl}amino)thieno[3,2-d]pyrimidin-6-yl]prop-2-ynyl(methyl)carbamate hydrochloride;
N-{3-chloro-4-[(3-fluorobenzyl)oxy]phenyl}-6-[3-(dimethylamino)prop-1-ynyl]thieno[2,3-d]pyrimidin-4-amine;
tert-butyl 3-[4-({3-chloro-4-[(3-fluorobenzyl)oxy]phenyl}amino)thieno[2,3-d]pyrimidin-6-yl]prop-2-ynylcarbamate;
N-{3-chloro-4-[(3-fluorobenzyl)oxy]phenyl}-6-[3-(isopropylamino)prop-1-ynyl]thieno[3,2-d]pyrimidin-4-amine;
N-{3-chloro-4-[(3-fluorobenzyl)oxy]phenyl}-6-(3-{methyl[2-(methylsulfonyl)ethyl]amino}prop-1-ynyl)thieno[3,2-d]pyrimidin-4-amine;
N-{3-chloro-4-[(3-fluorobenzyl)oxy]phenyl}-6-[3-(dipropylamino)prop-1-ynyl]thieno[3,2-d]pyrimidin-4-amine hydrochloride;
N-{3-chloro-4-[(3-fluorobenzyl)oxy]phenyl}-6-[3-(diisobutylamino)prop-1-ynyl]thieno[3,2-d]pyrimidin-4-amine;
6-(3-aminoprop-1-ynyl)-N-{3-chloro-4-[(3-fluorobenzyl)oxy]phenyl}thieno[2,3-d]pyrimidin-4-amine;
N-{3-chloro-4-[(3-fluorobenzyl)oxy]phenyl}-6-(pyrimidin-2-ylethynyl)thieno[3,2-d]pyrimidin-4-amine;
6-[3-(benzylamino)prop-1-ynyl]-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-(pyrimidin-2-ylethynyl)thieno[2,3-d]pyrimidin-4-amine;
N-{3-chloro-4-[(3-fluorobenzyl)oxy]phenyl}-6-[3-(methylamino)prop-1-ynyl]thieno[2,3-d]pyrimidin-4-amine;
N-{3-chloro-4-[(3-fluorobenzyl)oxy]phenyl}-6-{3-[(pyridin-4-ylmethyl)amino]prop-1-ynyl}thieno[3,2-d]pyrimidin-4-amine;
N-{3-chloro-4-[(3-fluorobenzyl)oxy]phenyl}-6-(pyridin-2-ylethynyl)thieno[2,3-d]pyrimidin-4-amine;
N-{3-chloro-4-[(3-fluorobenzyl)oxy]phenyl}-6-(pyridin-2-ylethynyl)thieno[3,2-d]pyrimidin-4-amine;
N-{3-[4-({3-chloro-4-[(3-fluorobenzyl)oxy]phenyl}amino)thieno[2,3-d]pyrimidin-6-yl]prop-2-ynyl}-N′-[2-(methylsulfonyl)ethyl]urea;
N-{3-[4-({3-chloro-4-[(3-fluorobenzyl)oxy]phenyl}amino)thieno[2,3-d]pyrimidin-6-yl]prop-2-ynyl}-N′-(2-cyanoethyl)urea;
N′-{3-[4-({3-chloro-4-[(3-fluorobenzyl)oxy]phenyl}amino)thieno[2,3-d]pyrimidin-6-yl]prop-2-ynyl}-N,N-dimethylurea;
N-{3-[4-({3-chloro-4-[(3-fluorobenzyl)oxy]phenyl}amino)thieno[2,3-d]pyrimidin-6-yl]prop-2-ynyl}-N′-[2-(dimethylamino)ethyl]urea;
N-{3-chloro-4-[(3-fluorobenzyl)oxy]phenyl}-6-[3-(diisopentylamino)prop-1-ynyl]thieno[3,2-d]pyrimidin-4-amine hydrochloride;
N-{3-chloro-4-[(3-fluorobenzyl)oxy]phenyl}-6-[3-(1,1-dioxidothiomorpholin-4-yl)prop-1-ynyl]thieno[2,3-d]pyrimidin-4-amine;
N-{3-[4-({3-chloro-4-[(3-fluorobenzyl)oxy]phenyl}amino)thieno[2,3-d]pyrimidin-6-yl]prop-2-ynyl}urea;
N-{3-[4-({3-chloro-4-[(3-fluorobenzyl)oxy]phenyl}amino)thieno[2,3-d]pyrimidin-6-yl]prop-2-ynyl}acetamide;
N-{3-chloro-4-[(3-fluorobenzyl)oxy]phenyl}-6-(1,3-thiazol-2-ylethynyl)thieno[3,2-d]pyrimidin-4-amine;
N-{3-chloro-4-[(3-fluorobenzyl)oxy]phenyl}-6-(3-piperidin-1-ylprop-1-ynyl)thieno[3,2-d]pyrimidin-4-amine;
5-{[4-({3-chloro-4-[(3-fluorobenzyl)oxy]phenyl}amino)thieno[3,2-d]pyrimidin-6-yl]ethynyl}-2-furaldehyde;
N-{3-[4-({3-chloro-4-[(3-fluorobenzyl)oxy]phenyl}amino)thieno[2,3-d]pyrimidin-6-yl]prop-2-ynyl}-2-pyridin-4-ylacetamide;
N-{3-[4-({3-chloro-4-[(3-fluorobenzyl)oxy]phenyl}amino)thieno[2,3-d]pyrimidin-6-yl]prop-2-ynyl}-2-pyridin-2-ylacetamide;
N-{3-[4-({3-chloro-4-[(3-fluorobenzyl)oxy]phenyl}amino)thieno[2,3-d]pyrimidin-6-yl]prop-2-ynyl}-2-(1-methyl-1H-imidazol-4-yl)acetamide;
N-{3-[4-({3-chloro-4-[(3-fluorobenzyl)oxy]phenyl}amino)thieno[2,3-d]pyrimidin-6-yl]prop-2-ynyl}-2-thien-3-ylacetamide;
N-{3-[4-({3-chloro-4-[(3-fluorobenzyl)oxy]phenyl}amino)thieno[2,3-d]pyrimidin-6-yl]prop-2-ynyl}cinnoline-4-carboxamide;
3-[4-({3-chloro-4-[(3-fluorobenzyl)oxy]phenyl}amino)thieno[3,2]pyrimidin-6-yl]prop-2-ynyl methanesulfonate;
N-{3-chloro-4-[(3-fluorobenzyl)oxy]phenyl}-6-(1,3-thiazol-2-ylethynyl)thieno[2,3-d]pyrimidin-4-amine;
6-(3-amino-3-methylbut-1-ynyl)-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-[3-(ethylamino)prop-1-ynyl]thieno[3,2-d]pyrimidin-4-amine;
3-({3-[4-({3-chloro-4-(3-fluorobenzyl)oxy]phenyl}amino)thieno[3,2-d]pyrimidin-6-yl]prop-2-ynyl}amino)propanenitrile;
N-{3-chloro-4-[(3-fluorobenzyl)oxy]phenyl}-6-{3-[(2-methoxyethyl)amino]prop-1-ynyl}thieno[3,2-d]pyrimidin-4-amine;
N-{3-chloro-4-[(3-fluorobenzyl)oxy]phenyl}-6-[3-(propylamino)prop-1-ynyl]thieno[3,2-d]pyrimidin-4-amine;
N-{3-chloro-4-[(3-fluorobenzyl)oxy]phenyl}-6-{[5-({[2-(methylsulfonyl)ethyl]amino}methyl)-2-furyl]ethynyl}thieno[3,2-d]pyrimidin-4-amine;
N-{3-chloro-4-[(3-fluorobenzyl)oxy]phenyl}-6-(3-}[2-(1H-imidazol-4-yl)ethyl]amino}prop-1-ynyl)thieno[3,2-d]pyrimidin-4-amine;
4-[4-({3-chloro-4-[(3-fluorobenzyl)oxy]phenyl}amino)thieno[3,2-d]pyrimidin-6-yl]-2-methylbut-3-yn-2-ol;
N-{3-chloro-4-[(3-fluorobenzyl)oxy]phenyl}-6-(1H-imidazol-4-ylethynyl)thieno[3,2-d]pyrimidin-4-amine;
4-[4-({3-chloro-4-[(3-fluorobenzyl)oxy]phenyl}amino)thieno[3,2-d]pyrimidin-6-yl]but-3-yn-1-ol;
N-{3-chloro-4-[(3-fluorobenzyl)oxy]phenyl}-6-(phenylethynyl)thieno[3,2-d]pyrimidin-4-amine;
N-{3-chloro-4-[(3-fluorobenzyl)oxy]phenyl}-6-{[6-({[2-(methylsulfonyl)ethyl]amino}methyl)pyridin-2-yl]ethynyl}thieno[2,3-d]pyrimidin-4-amine hydrochloride; and
6-{[4-({3-chloro-4-[(3-fluorobenzyl)oxy]phenyl}amino)thieno[2,3-d]pyrimidin-6-yl]ethynyl}pyridine-2-carbaldehyde.

Additionally provided in the present invention are the following compounds:
(R,S)-6-(3-aminobut-1-ynyl)-N-{3-chloro-4-[(3-fluorobenzyl)oxy]phenyl}thieno[3,2-d]pyrimidin-4-amine;
(R)-6-(3-Aminobut-1-ynyl)-N-{3-chloro-4-[(3-fluorobenzyl)oxy]phenyl}thieno[3,2-d]pyrimidin-4-amine;
(S)-6-(3-Aminobut-1-ynyl)-N-{3-chloro-4-[(3-fluorobenzyl)oxy]phenyl}thieno[3,2-d]pyrimidin-4-amine;
(R,S)-6-(3-Aminopent-1-ynyl)-N-{3-chloro-4-[(3-fluorobenzyl)oxy]phenyl}thieno[3,2-d]pyrimidin-4-amine;
(R,S)-6-(3-aminobut-1-ynyl)-N-[3-chloro-4-(1-naphthyloxy)phenyl]thieno[3,2-d]pyrimidin-4-amine;
(R,S)-6-(3-aminobut-1-ynyl)-N-(2-benzyl-1H-benzimidazol-5-yl)thieno[3,2-d]pyrimidin-4-amine;
(R,S)-6-(3-aminobut-1-ynyl)-N-[1-(pyridin-3-ylmethyl)-1H-indol-5-yl]thieno[3,2-d]pyrimidin-4-amine;
(R,S)-N⁴-[6-(3-aminobut-1-ynyl)thieno[3,2-d]pyrimidin-4-yl]-2-chloro-N¹-(3-fluorobenzyl)benzene-1,4-diamine;
(R,S)-6-(3-aminobut-1-ynyl)-N-[1-(3-fluorobenzyl)-1H-indazol-5-yl]thieno[3,2-d]pyrimidin-4-amine;
(R,S)-6-(3-aminobut-1-ynyl)-N-{3-fluoro-4-[(3-fluorobenzyl)oxy]phenyl}thieno[3,2d]pyrimidin-4-amine;
(R,S)-6-(3-Aminobut-1-ynyl)-N-(4-benzylphenyl)thieno[3,2-d]pyrimidin-4-amine;
(R,S)-6-(3-Aminobut-1-ynyl)-N-[1-(2-fluorobenzyl)-1H-indazol-5-yl]thieno[3,2-d]pyrimidin-4-amine;
(R,S)-6-(3-Aminobut-1-ynyl)-N-[2-(2-fluorobenzyl)-1H-benzimidazol-5-yl]thieno[3,2d]pyrimidin-4-amine;
(R,S)-6-(3-aminobut-1-ynyl)-N-[1-(2,5-difluorobenzyl)-1H-indol-5-yl]thieno[3,2-d]pyrimidin-4-amine;
(R,S)-6-(3-aminobut-1-ynyl)-N-(1-benzyl-1H-indol-5-yl)thieno[3,2-d]pyrimidin-4-amine;
(R,S)-6-(3-aminobut-1-ynyl)-N-(1-benzyl-1H-indazol-5-yl)thieno[3,2-d]pyrimidin-4-amine;
(R,S)-6-(3-aminobut-1-ynyl)-N-[2-(3-fluorobenzyl)1H-benzimidazol-5-yl]thieno[3,2-d]pyrimidin-4-amine;
(2R,S)-N-{3-Chloro-4-[(3-fluorobenzyl)oxy]phenyl}-6-{3-[(2-methoxyethyl)amino]but-1-ynyl}thieno[3,2-d]pyrimidin-4-amine;
(2R)-2-amino-4-[4-({3-chloro-4-[(3-fluorobenzyl)oxy]phenyl}amino)thieno[3,2-d]pyrimidin-6-yl]but-3-yn-1-ol; and
(2S)-2-amino-4-[4-({3-chloro-4-[(3-fluorobenzyl)oxy]phenyl}amino)thieno[3,2-d]pyrimidin-6-yl]but-3-yn-1-ol.

The compounds according to the invention may contain one or more asymmetric atoms and thus occur as racemates, racemic mixtures, single enantiomers, diastereomeric mixtures and individual diastereoisomers. All such isomeric forms of these compounds are expressly included in the present invention. Each stereogenic atom may be of the R or S configuration. Although the specific compounds exemplified in this application may be depicted in a particular stereochemical configuration, compounds having either the opposite stereochemistry at any given chiral center or mixtures thereof are also envisioned.

As recited above n is 0-6. It is understood that each n is independently selected.

The term “alkyl”, alone or in combination with any other term, refers to a straight-chain or branched-chain saturated aliphatic hydrocarbon radical containing the specified number of carbon atoms, optionally substituted with hydroxy. Examples of alkyl radicals include, but are not limited to, methyl, hydroxymethyl, ethyl, hydroxyethyl, n-propyl, isopropyl, n-butyl, isobutyl, sec-butyl, tert-butyl, pentyl, isoamyl, n-hexyl and the like.

The term “alkylene,” alone or in combination with any other term, refers to a saturated aliphatic hydrocarbon radical in which the carbon atom(s) are generally substituted with zero, one, or two hydrogen atoms. An example of an alkylene radical is methylene, —CH₂—.

The terms “alkenyl” or “alkenylene” alone or in combination with any other term, refers to a straight-chain or branched-chain alkyl group with at least one carbon-carbon double bond. Examples of alkenyl and alkenylene radicals include, but are not limited to, ethenyl, propenyl, isopropenyl, butenyl, isobutenyl, pentenyl, hexenyl, hexadienyl and the like.

The term “alkynyl” refers to hydrocarbon groups of either a straight or branched configuration with one or more carbon-carbon triple bonds which may occur in any stable point along the chain, such as ethynyl, propynyl, butynyl, pentynyl, and the like.

The term “alkoxy” refers to alkyl ether radical, wherein the term “alkyl” is defined above. Examples of suitable alkyl ether radicals include, but are not limited to, methoxy, ethoxy, n-propoxy, isopropoxy, n-butoxy, isobutoxy, sec-butoxy, tert-butoxy and the like.

The term “cycloalkyl” refers to a saturated or partially saturated carbocyclic ring composed of 3-8 carbons in any chemically stable configuration. Examples of suitable carbocyclic groups include cyclopropyl, cyclobutyl, cyclopentyl, cyclohexyl, and cyclohexenyl.

The terms “aryl” refers to a carbocyclic aromatic moiety (such as phenyl or naphthyl) containing the specified number of carbon atoms, preferably from 6-14 carbon atoms, and more preferably from 6-10 carbon atoms. Examples of aryl radicals include, but are not limited to phenyl, naphthyl, indenyl, indanyl, azulenyl, fluorenyl, anthracenyl and the like.

As used herein, the term “heteroaryl” refers to a monocyclic five to seven membered aromatic ring, or to a fused bicyclic or tricyclic aromatic ring system comprising two of such monocyclic five to seven membered aromatic rings. These heteroaryl rings contain one or more nitrogen, sulfur, and/or oxygen heteroatoms, where N-oxides and sulfur oxides and dioxides are permissible heteroatom substitutions and may be optionally substituted with up to three members selected from a group consisting of C₁₋C₆ alkyl, C₁₋C₆ alkoxy, C₁₋C₆alkylsulfanyl, C₁₋C₆ alkylsulfenyl, C₁₋C₆ alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halo, C₁₋C₆ perfluoroalkyl, heteroaryl, or aryl, multiple degrees of substitution being allowed. Examples of “heteroaryl” groups used herein include, but are not limited to, furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolyl, quinolinyl, isoquinolyl, isoquinolinyl, benzofuranyl, benzothiophenyl, indolyl, indazolyl, indazolinyl, and substituted versions thereof.

The term “heterocycle,” “heterocyclic,” and “heterocyclyl” as used herein, refer to a non-aromatic 3- to 7- membered monocyclic heterocyclic ring or 8-to 11- membered bicyclic heterocyclic ring which is either saturated, partially saturated or unsaturated, and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and including any bicyclic group in which any of the above-defined heterocyclic rings is fused to a benzene ring. The heterocyclic ring may be attached at any carbon or heteroatom, which results in the creation of a stable structure. Preferred heterocycles include 5-7 membered monocyclic heterocycles and 8-10 membered bicyclic heterocycles. Examples of such groups include, but are not limited to tetrahydrofuranyl, 1,4-dioxanyl, 1,3-dioxanyl, piperidinyl, piperazinyl, 2,4-piperazinedionyl, pyrrolidinyl, imidazolidinyl, pyrazolidinyl, morpholinyl, thiomorpholinyl, tetrahydrothiopyranyl, tetrahydrothiophenyl, benzodioxyl, and the like as well as additional substituted versions thereof.

The term “halogen” refers to fluorine, chlorine, bromine or iodine.

Compounds of formula (I) may be subdivided into compounds of formulae (II) and (III), shown below.

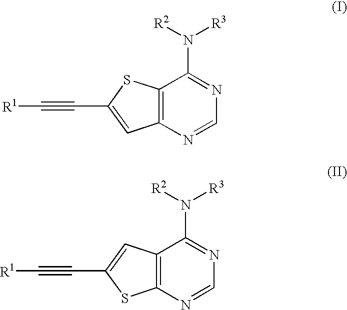

Compounds of general formulae (II) and (III) may be prepared by methods known to those of skill in the art. The following synthetic schemes are meant to represent examples only and are not meant to limit the invention in any way. In all of the schemes described below, it is understood that protecting groups may be employed where necessary in accordance with general principles known to those of skill in the art, for example, see T. W. Green and P. G. M. Wuts (1991) Protecting Groups in Organic Synthesis, John Wiley & Sons. These groups may be removed at a convenient stage of the compound synthesis using methods known those of skill in the art. The selection of processes as well as the reaction conditions and order of their execution shall be consistent with the preparation of compounds of formulae (II) and (III). Those of skill in the art will recognize that if a stereocenter exists in compounds of Formulas (II) and (III), the present invention is meant to include both enantiomers, mixtures of such enantiomers and the individual enantiomers substantially free of the opposite enantiomer. In addition, when a compound contains more than one stereocenter, one of skill in the art will recognize that the present invention is meant to include mixtures of diastereomeric compounds, mixtures of enantiomers and the individual enantiomers substantially free of the opposite enantiomer.

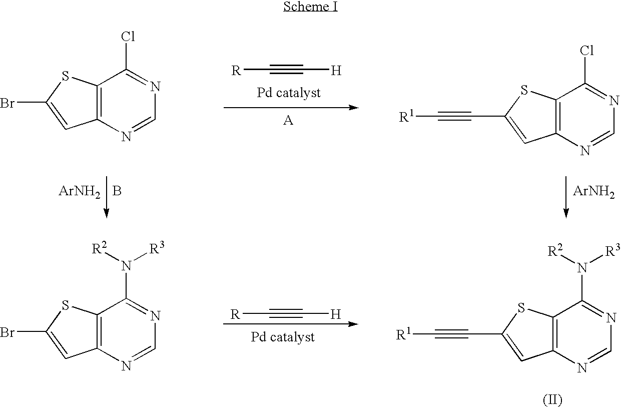

The compounds of formula (II), wherein R¹, R² and R³ are defined as above, may be prepared from the appropriate hal-substituted trienopyrimidine by the general synthetic routes depicted as A and B shown below in Scheme (I). In step 1 of route A, the hal-substituted thienopyrimidine is coupled with a terminal acetylenic compound. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C. The resulting product may then be allowed to react with an arylamine to displace the 6-chloro substituent on the pyrimidine moiety. These displacement reactions are typically performed in a solvent, isopropanol for example, and at a temperature from 25° C. to 175° C., preferably 50° C. to 80° C.

Alternatively, the compounds of formula (II) may be prepared by carrying out the displacement and coupling steps described above in reverse order using similar conditions.

The appropriate halogen-substituted thienopyrimidines are either commercially available or may be prepared using methods known to those of skill in the art. For example, 6-bromo-4-chlorothieno[3,2-d]pyrimidine may be prepared by the procedure described in published PCT application number WO 99/24440.

The acetylenyl reagents are either commercially available or can be prepared by methods known to those skilled in the art. For example, see Gilbert et al. (J. Org. Chem., 1982, 47, 1837) and Dinersterin et al. (US Pat. No. 5,409,492).

The arylamines are either commercially available or can be prepared by methods known to those skilled in the art. For example, seethe methods described in U.S. Pat. Nos. 6,174, 883 B1 and 6,207,669 B1, which are hereby incorporated by reference.

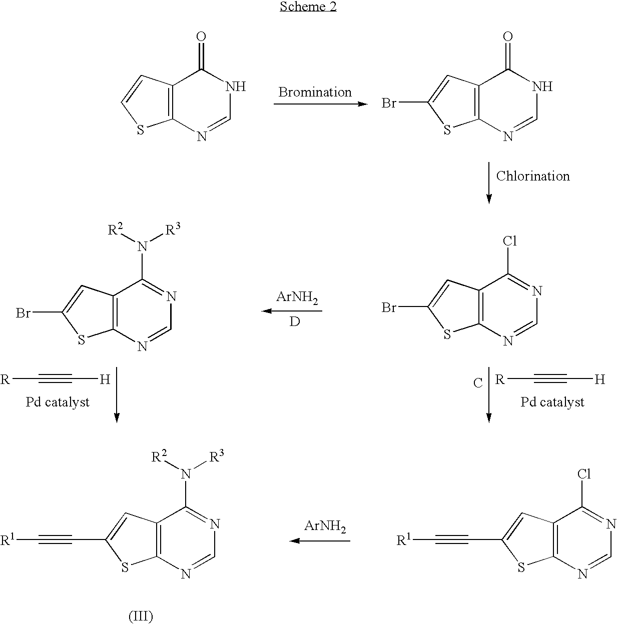

The compounds of the general structure (III) wherein R¹, R² and R³ are defined as above may be prepared by the procedure shown below in Scheme (2). In the first step, commercially available (Maybridge Chemical Co.) thieno[2,3-d]pyrimid-4(1H)one is allowed to react with a brominating agent to afford 6-bromo-thieno[2,3d]pyrimid-4(1H)-one. These reactions are generally performed in the presence of a brominating reagent such as bromine, a solvent, acetic acid for example, and at a temperature from 25° C. to 175° C., preferably 60° C. to 100° C.

Next, a substituent capable of acting as a leaving group, chlorine for example, is introduced into the pyrimidine portion of the 6-thienopyrmidine intermediate. The leaving group may be introduced using a reagent capable of reacting selectively with the pyrimidine portion of the molecule, phosphorous oxychloride for example, to afford an appropriately substituted product, These reactions are generally performed at a temperature from 25° C. to 175° C., preferably 80° C. to 106° C. For example, 6-bromo-thieno[2,3-d]pyrimid-4(1H)one was allowed to react with phosphorus oxychloride at 106° C. to afford 6-bromo-4-chlorothieno[2,3-d]pyrimidine.

The intermediate dihalogenated thieno[2,3-d]pyrimidines can then be converted to compounds of the general structure (III) by the two synthetic routes depicted as C and D in Scheme 2. In the first step of route C, an appropriate dihalogenated thieno[2,3-d]pyrimidine is allowed to react with reagents capable of selectively introducing an acetylenyl group into the 6-position. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C.

Lastly, the resulting alkyne is allowed to react with an arylamine to displace the 6-chloro substituent on the pyrimidine moiety as described above for step 2 of Scheme 1. These reactions are generally performed in a solvent, isopropanol for example, and at a temperature from 25° C. to 175° C., preferably 50° C. to 80° C.

The acetylenyl reagents are either commercially available or can be prepared by methods known to those skilled in the art. For example, see Gilbert et al. (J. Org. Chem., 1982, 47, 1837) and Dinersterin et al., U.S. Pat. No. 5,409,492, which is hereby incorporated by reference.

The arylamines are either commercially available or can be prepared by methods known to those skilled in the art. For example, see the methods described in U.S. Pat. Nos. 6,174,883 B1 and 6,207,669 B1, which are hereby incorporated by reference.

Alternatively, steps C and D in Scheme 2 may be carried out in reverse order using similar conditions as described above to afford the desired products.

Following the steps outlined in Schemes 1 and 2, the R¹ group of compounds of formula (II) and formula (III) may be further modified to prepare compounds of formula (I), wherein R¹ is —(CR¹¹R¹¹)ₙ—R⁵, wherein R⁵ is selected from the group consisting of heteroaryl optionally substituted with one or more substituents selected from the group consisting of halo, —CF₃, C₁₋₆alkoxy, —NO₂, C₁₋₆alky, —CN, —S(O)₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alky, —CN, —S(O)₂R⁹, and —(CH₂)ₙNR⁹R¹⁰. In this process, the acetylenyl reagent employed is a suitably protected acetylene derivative, such as commercially available trimethylsilylacetylene (R¹=TMS). The coupling of such a suitably protected acetylene derivative would yield compound of formula (IV) in Scheme 3. Further elaboration by deprotection, for example by treatment with tetrabutylammonium fluoride, yields compounds of formula (V). Such deprotection reactions are generally performed in a solvent, tetrahydofuran for example, and at a temperature from 0° C. to 100° C., preferably 0° C. to 25° C. Compounds of formula (V) may be coupled with halogen substituted heteroaryl or aryl compounds to provide the desired heteroaryl derivatives. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C. The halogen substituted heteroaryl and aryl compounds are either commercially available or can be prepared by methods known to those skilled in the art.

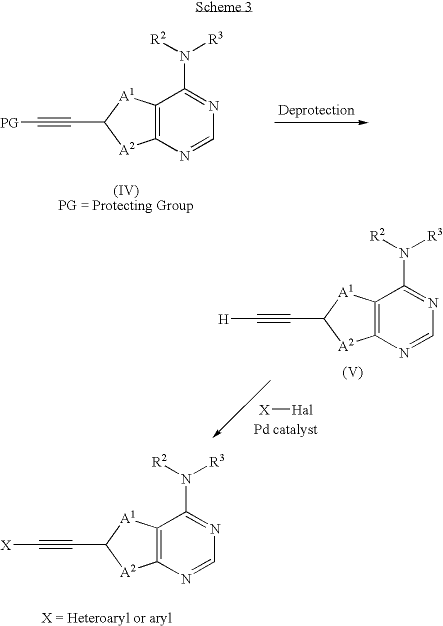

Compounds of formula (I), wherein R¹ is —(CR¹¹R¹¹)ₙ—R⁵, R⁵ is heteroaryl optionally substituted with one or more substituents selected from the group consisting of halo, —CF₃, C₁₋₆alkoxy, —NO₂, C₁₋₆alky, —CN, —S(O)₂R⁹, and —(CH₂)ₙNR⁹R¹⁰, or aryl optionally substituted with one or more substituents selected from the group consisting of halo, —CF₃, C₁₋₆alkoxy, —NO₂, C₁₋₆alky, —CN, —S(O)₂R⁹, and —(CH₂)ₙNR⁹R¹⁰, and R⁹ and R¹⁰ are as hereinbefore defined, may be prepared from an appropriately substituted 6-halotheinopyrimidine derivative, such as those shown in Schemes 1 and 2, by reaction with an appropriately substituted heteroaryl or aryl acetylene derivative, for example, commercially available 3-phenyl-1-propyne. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C. The appropriately substituted heteroaryl or aryl acetylene derivatives are either commercially available or may be prepared using methods known to those of skill in the art.

Compounds of formula (I), wherein R¹ is —(CR¹¹R¹¹)ₙ—R⁵, R⁵ is C(O)NR⁶R⁷, and R⁶ and R⁷ are as hereinbefore defined, may be prepared using an appropriately substituted 6-halotheinopyrimidine derivative, such as those shown in Schemes 1 and 2, by reaction with an appropriately substituted alkyne carboxylic acid derviative, for example commercially available propiolic acid. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C. The resulting carboxylic acid derivative may then be allowed to react with an appropriately substituted amine to afford the desired compounds. These reactions are generally performed in the presence of a coupling reagent, diethyl cyanophosphonate for example, and a base, triethylamine for example, in a solvent, dimethylformamide for example, and at a temperature from 25° C. to 175° C., preferably 25° C.

The appropriately substituted alkyne carboxylic acid derviatives are either commercially available or may be prepared using methods known to those of skill in the art.

The appropriately substituted amines are either commercially available or may be prepared by methods known to those of skill in the art. Compounds of formula (I), wherein R¹ is —(CR¹¹R¹¹)ₙ—R⁵, R⁵ is heterocyclyl, —(CH₂)ₙNR⁶R⁷, —(CH₂)ₙOR⁷, or —(CH₂)ₙSR⁸, and wherein R⁶, R⁷, and R⁸ are as hereinbefore defined, may be prepared from compounds of formula (I), wherein R⁵ is —(CH₂)ₙOH. In one process, the alcohol functionality may be converted to a leaving group known by those of skill in the art to be suitable, for example mesylate as shown in Scheme 4.

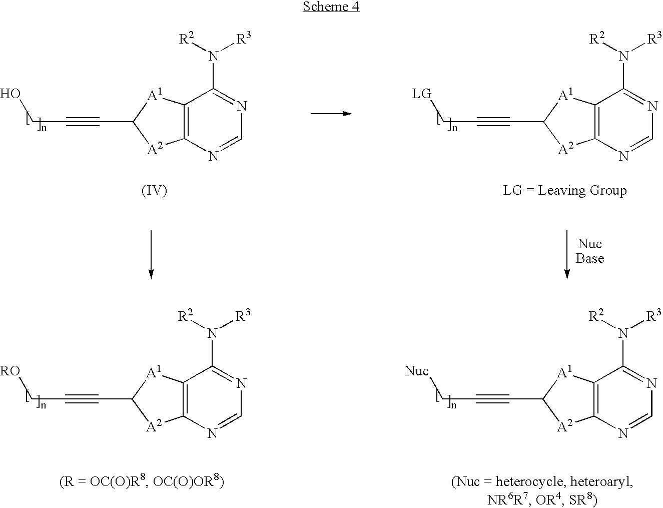

These reactions are generally performed using a mesylating reagent, methanesufonic anhydride or methansulfonyl chloride for example, in the presence of a base, diethylisopropylamine for example, in a solvent, N,N-dimethylacetamide for example, and at a temperature from 0° C. to 175° C., preferably 60° C. to 100° C. The mesylate leaving group may then be displaced by an appropriate nucleophilic heterocycle, amine, alcohol or thiol containing compound. These displacement reactions are generally performed in the presence of a suitable base, diethylisopropylamine for example, in a solvent, N,N-dimethylacetamide for example, and at a temperature from 0° C. to 175° C., preferably 60° C. to 100° C. as shown in Scheme 4.

The appropriate heterocycle, amine, alcohol or thiol containing compounds are either commercially available or can be prepared by methods known to those skilled in the art.

Compounds of formula (I), wherein R¹ is —(CR¹¹R¹¹)ₙ—R⁵, R⁵ is —(CH₂)ₙS(O)R⁸ or —(CH₂)ₙS(O)₂R⁸, and R⁸ is as hereinbefore defined, may be prepared from compounds of formula (I), wherein R¹ is —(CR¹¹R¹¹)ₙ—R⁵, and R⁵ is —(CH₂)ₙSR⁸ by reaction with a reagents or reagents capable of selectively oxidizing the thiol functionality. These reactions are generally performed using an oxidizing reagent, metachloroperbenzoic for example, in a solvent, ether for example, and at a temperature from 0° C. to 175° C., preferably 0° C. to 25° C. Compounds of formula (I), wherein R¹ is —(CR¹¹R¹¹)ₙ—R⁵, R⁵ is —OC(O)R⁸ and R⁸ is as hereinbefore defined, may be prepared from compounds of formula (I), wherein R¹ is —(CR¹¹R¹¹)ₙ—R⁵, and R⁵ is —(CH₂)ₙOH by reaction with an appropriate carboxylic acid. These reactions are generally performed in the presence of a coupling reagent, diethyl cyanophosphonate for example, and a base, triethylamine for example, in a solvent, dimethylformamide for example, and at a temperature from 25° C. to 175° C., preferably 25° C.

The appropriate carboxylic acids are either commercially available or may be prepared by methods known to those of skill in the art. Compounds of formula (I), wherein R¹ is —(CR¹¹R¹¹)ₙ—R⁵, R⁵ is —OC(O)OR⁸ and R⁸ is as hereinbefore defined, may be prepared from compounds of formula (I), wherein R¹ is —(CR¹¹R¹¹)ₙ—R⁵, and R⁵ is —(CH₂)ₙOH by reaction with an appropriate chloroformate. These reactions are generally performed in the presence of a base, pyridine for example, in a solvent, dichloromethane for example, and at a temperature from 25° C. to 175° C., preferably 25° C.

The appropriate chloroformates are either commercially available or may be prepared by methods known to those of skill in the art.

Compounds of formula (I), wherein R¹ is —(CR¹¹R¹¹)ₙ—R⁵, and R⁵ is —(CH₂)ₙOH, may be prepared from an appropriately substituted 6-halotheinopyrimidine derivative, such as those shown in Schemes 1 and 2, by reaction with an appropriately substituted hydroxy alkyne derviative, such as commercially available propargyl alcohol. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C.

Compounds of formula (I), wherein R¹ is —(CR¹¹R¹¹)ₙ—R⁵, R⁵ is N(R⁶)—C(O)—N(R⁶)(R⁷), and R⁶ and R⁷ are as hereinbefore defined may be prepared from compounds of formula (I), wherein R¹ is —(CR¹¹R¹¹)ₙ—R⁵, R⁵ is —NR⁶R⁷, wherein R⁶ is as hereinbefore defined and R⁷ is hydrogen, by reaction with an appropriate isocyanate. These reactions are generally performed in a solvent, chloroform for example, and at a temperature from 25° C. to 175° C., preferably 25° C. Alternatively, the amine may be allowed to react with 1,1′-Alterantively, the isocyanate may be formed in situ, by reaction of the amine with an agent capable of forming the isocyanate, carbonyl diimidazole for example, followed by reaction with a second amine. These reactions are generally performed in a solvent, N,N-dimethylacetamide for example, and at a temperature from 25° C. to 175° C., preferably 25° C.

The appropriate isocyantes and amines are either commercially available or can be prepared using methods known to those of skill in the art. Compounds of formula (I) wherein R⁵ is N(R⁶)—C(S)—N(R⁶)(R⁷), and R⁶ and R⁷ are as hereinbefore defined, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with an appropriate isothiocyanate. These reactions are generally performed in a solvent, chloroform for example, and at a temperature from 25° C. to 175° C., preferably 25° C.

The appropriate isothiocyanates are either commerically available or can be prepared using methods known to those of skill in the art.

Compounds of formula (I), wherein R¹ is —(CR¹¹R¹¹)ₙ—R⁵, R⁵ is —N(R⁶)—C(O)—OR⁷, and R⁶ and R⁷ are as hereinbefore defined, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with an appropriate chloroformate. These reactions are generally performed in the presence of a base, pyridine for example, in a solvent, dichloromethane for example, and at a temperature from 25° C. to 175° C., preferably 25° C.

Alternatively, compounds of formula (I), wherein R¹ is —(CR¹¹R¹¹)ₙ—R⁵, R⁵ is —N(R⁶)—C(O)—OR⁷, and R⁶ and R⁷ are as hereinbefore defined, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with a reagent capable of forming an isocyanate in situ, carbonyl diimidazole for example, followed by reaction with an appropriate alcohol. These reactions are generally performed in a solvent, N,N-dimethylacetamide for example, and at a temperature from 25° C. to 175° C., preferably 25° C.

Compounds of formula (I), wherein R¹ is —(CR¹¹R¹¹)ₙ—R⁵, R⁵ is —N(R⁶)—C(O)R⁷, wherein R⁶ and R⁷ are as hereinbefore defined, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with an appropriate carboxylic acid. These reactions are generally performed in the presence of a coupling reagent, diethyl cyanophosphonate for example, and a base, triethylamine for example, in a solvent, dimethylformamide for example, and at a temperature from 25° C. to 175° C., preferably 25° C.

The carboxylic acids are either commercially available or may be prepared by methods known to those of skill in the art.

Alterantively, compounds of formula (I), wherein R¹ is —(CR¹¹R¹¹)ₙ—R⁵, R⁵ is —N(R⁶)—C(O)R⁷, wherein R⁶ and R⁷ are as hereinbefore defined, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with an appropriate acid chloride. These reactions are generally performed in the presence of a base, pyridine for example, in a solvent, dichloromethane for example, and at a temperature from 25° C. to 175° C., preferably 25° C.

The appropriate acid chlorides are either commercially available or may be prepared by methods known to those of skill in the art. Compounds of formula (I), wherein R¹ is —(CR¹¹R¹¹)ₙ—R⁵, R⁵ is —NR⁶R⁷, R⁶ is as hereinbefore defined and R⁷ is hydrogen may be prepared from appropriately substituted 6-halotheinopyrimidine derivative, such as those shown in Schemes 1 and 2, by reaction with an appropriately substituted amine substituted alkyne derviative. These reactions are generally performed in the presence of a palladium catalyst, bis(triphenylphosphine)palladium dichloride for example, a copper catalyst, copper(I) iodide for example, a base, triethylamine for example, a solvent, tetrahydrofuran (THF) for example, and at a temperature from 25° C. to 175° C., preferably 50° C. to 60° C. Generally, the amine substituted alkune derivative is first protected with a suitable protecting group, such as the tert-butyloxycarbonyl. These protections are generally performed using a suitable protecting group, tert-butyloxycarbonyl anhydride for example, in a solvent, THF for example, and at a temperature from 0° C. to 175° C., preferably 0° C. to 25° C. Such suitably protected alkynamines are then used as the acetylenyl reagent for coupling with the substituted 6-halotheinopyrimidine derivative as shown in Schemes 1 and 2. Subsequent deprotection of the amine affords the desired compounds of formula (I), wherein R¹ is —(CR¹¹R¹¹)ₙ—R⁵, R⁵ is —NR⁶R⁷, R⁶ is as hereinbefore defined and R⁷ is hydrogen.

The appropriate amine substituted alkyne derivatives are either commercially available or may be prepared using methods known to those of skill in the art.

Compounds of formula (I), wherein R¹ is —(CR¹¹R¹¹)ₙ—R⁵, R⁵ is —NR⁶R⁷, R⁶ is as hereinbefore defined and R⁷ is C₁₋₆alkyl, may be prepared from compounds of formula (I), wherein R¹ is —(CR¹¹R¹¹)ₙ—R⁵, R⁵ is —NR⁶R⁷, R⁶ is as hereinbefore defined and R⁷ is hydrogen, by reaction with an appropriate aldehyde, and in the presence of a reducing agent, such as sodium triacetoxyborohydride. These reactions are generally performed in a solvent, ethanol and acetic acid for example, and at a temperature from 25° C. to 175° C., preferably 25° C.

The appropriate aldehydes are either commercially available or may be prepared by methods known to those of skill in the art.

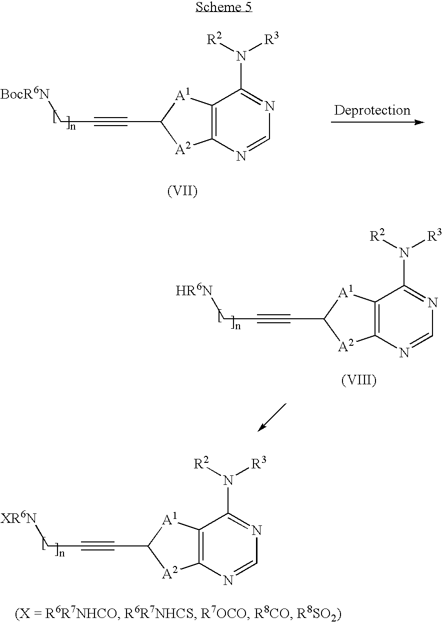

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the anticancer agent.

As used herein, the compounds according to the invention are defined to include pharmaceutically acceptable derivatives thereof. A “pharmaceutically acceptable derivative” means any pharmaceutically acceptable salt, ester, salt of an ester, or other derivative of a compound of this invention which, upon administration to a recipient, is capable of providing (directly or indirectly) a compound of this invention or an inhibitorily active metabolite or residue thereof. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g. sodium), alkaline earth metal (e.g., magnesium), ammonium and NW₄⁺ (wherein W is C₁₋₄alkyl). Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of said compound in combination with a suitable cation such as Na⁺, NH₄⁺, and NW₄⁺ (wherein W is a C₁₋₄alkyl group).

Any reference to any of the above compounds also includes a reference to a pharmaceutically acceptable salt thereof.

While it is possible that, for use in therapy, therapeutically effective amounts of a compound of formula (I), or salts or anhydrate or hydrate forms thereof, may be administered as the raw chemical, it is possible to present the active ingredient as a pharmaceutical composition. Accordingly, the invention further provides pharmaceutical compositions, which include therapeutically effective amounts of compounds of the formula (I), or salts or anhydrate or hydrate forms thereof, and one or more pharmaceutically acceptable carriers, diluents, or excipients. The compounds of the formula (I), or salts or anhydrate or hydrate forms thereof, are as described above. The carrier(s), diluent(s) or excipient(s) must be acceptable in the sense of being compatible with the other ingredients of the formulation and not deleterious to the recipient thereof. According to another aspect of the invention there is also provided a process for the preparation of a pharmaceutical formulation including admixing a compound of formula (I), or salts or anhydrate or hydrate forms thereof, with one or more pharmaceutically acceptable carriers, diluents or excipients.

Compounds of formula (I), or salts or anhydrate or hydrate forms thereof, may be formulated for administration by any route, and the appropriate route will depend on the disease being treated as well as the subjects to be treated. Suitable pharmaceutical formulations include those for oral, rectal, nasal, topical (including buccal, sub-lingual, and transdermal), vaginal or parenteral (including intramuscular, sub-cutaneous, intravenous, and directly into the affected tissue) administration or in a form suitable for administration by inhalation or insufflation. The formulations may, where appropriate, be conveniently presented in discrete dosage units and may be prepared by any of the methods well know in the pharmacy art.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agents can also be present.

Capsules are made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I), or salts or anhydrate or hydrate forms thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I), or salts or anhydrate and hydrate forms thereof, may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation, through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insulators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may include other agents conventional in the art having regard to the type of formulation in question, for example those suitable for oral administration may include flavouring agents.

Also provided in the present invention, is a method for treating a disorder in a mammal characterized by aberrant activity of at least one erbB family PTK which includes administering a therapeutically effective amount of a compound of formula (I) or a salt thereof, to said mammal. The compounds of formula (I) and salts thereof are as described above.

The aberrant PTK activity referred to herein is any ErbB family PTK activity that deviates from the normal ErbB family protein kinase activity expected in a particular mammalian subject. Aberrant ErbB family PTK activity may take the form of, for instance, an abnormal increase in activity, or an aberration in the timing and or control of PTK activity. Such aberrant activity may result then, for example, from overexpression or mutation of the protein kinase leading to inappropriate or uncontrolled activation. Furthermore, it is also understood that unwanted PTK activity may reside in an abnormal source, such as a malignancy. That is, the level of PTK activity does not have to be abnormal to be considered aberrant, rather the activity derives from an abnormal source.

The compounds of formula (I) or salts thereof, are inhibitors of one or more ErbB family PTKs and as such have utility in the treatment of disorders in mammals which are characterized by aberrant PTK activity, particularly humans. In one embodiment of the present invention, the disorder treated is characterized by at least one ErbB family PTK, selected from EGFR, c-ErbB-2 and c-ErbB-4, exhibiting aberrant activity. In another embodiment, the disorder treated is characterized by at least two ErbB family PTKs, selected from EGFR, c-ErbB-2 and c-ErbB-4, exhibiting aberrant activity. In one embodiment of the treatment method, the compounds of formula (I) or salts thereof, inhibit at least one ErbB family PTK, selected from EGFR, c-ErbB-2 and c-ErbB-4. In another embodiment of the treatment method, the compounds of formula (I) or salts thereof inhibit at least two ErbB family PTKs selected from EGFR, c-ErbB-2 and c-ErbB-4.

Accordingly, also provided is a method of treating a disorder mediated by aberrant protein tyrosine kinase activity in a mammal, including: administering to said mammal an amount of a compound of formula (I) or salts thereof, effective to inhibit at least one ErbB family protein. In one embodiment, the method includes administering an amount of a compound of formula (I) or salts thereof, effective to inhibit at least two ErbB family proteins.

The disorders referred to may be any disorder which is characterized by aberrant PTK activity. As recited above such disorders include, but are not limited to, cancer and psoriasis. In a preferred embodiment, the disorder is cancer. In a more preferred embodiment, the cancer is non-small cell lung, bladder, prostate, brain, head and neck, breast, ovarian, gastric, colorectal, or pancreatic cancers.

The compounds of formula (I) and salts thereof have anticancer activity as demonstrated hereinafter by their inhibition of the protein tyrosine kinase c-ErbB-2, c-ErbB-4 and/or EGF-r enzymes and their effect on selected cell lines whose growth is dependent on c-ErbA-2 or EGF-r tyrosine kinase activity.

The present invention thus also provides compounds of formula (I) and pharmaceutically acceptable salts thereof for use in medical therapy, and particularly in the treatment of disorders mediated by aberrant protein tyrosine kinase activity such as human malignancies and the other disorders mentioned above. The compounds of the present invention are especially useful for the treatment of disorders caused by aberrant c-ErbB-2 and/or EGF-r activity such as breast, ovarian, gastric, pancreatic, non-small cell lung, bladder, head and neck cancers, and psoriasis.

A further aspect of the invention provides a method of treatment of a human or animal subject suffering from a disorder mediated by aberrant protein tyrosine kinase activity, including susceptible malignancies, which comprises administering to said subject an effective amount of a compound of formula (I) or a pharmaceutically acceptable salt or solvate thereof.

A further aspect of the present invention provides the use of a compound of formula (I), or a pharmaceutically acceptable salt or solvate thereof, in therapy.

A further aspect of the present invention provides the use of a compound of formula (I), or a pharmaceutically acceptable salt or solvate thereof, in the preparation of a medicament for the treatment of cancer and malignant tumors.

A further aspect of the present invention provides the use of a compound of formula (I), or a pharmaceutically acceptable salt thereof, in the preparation of a medicament for the treatment of psoriasis.

While it is possible for the compounds of the present invention, or salts or solvates thereof, to be administered as the new chemical, it is preferred to present them in the form of a pharmaceutical formulation.

According to a further feature of the present invention there is provided a pharmaceutical formulation comprising at least one compound of formula (I), or a pharmaceutically acceptable salt or solvate thereof, together with one or more pharmaceutically acceptable carriers, diluents or excipients.

A therapeutically effective amount of a compound of formula (I) or salts thereof, will depend on a number of factors including, but not limited to, the age and weight of the mammal, the precise disorder requiring treatment and its severity, the nature of the formulation, and the route of administration, and will ultimately be at the discretion of the attendant physcian or veternarian. Typically, the compounds of formula (I) or salts thereof, will be given for treatment in the range of 0.1 to 100 mg/kg body weight of recipient (mammal) per day and more usually in the range of 1 to 10 mg/kg body weight per day. Acceptable daily dosages, may be from about 0.1 to about 1000 mg/day, and preferably from about 0.1 to about 100 mg/day.

It should be understood that in addition to the ingredients particularly mentioned above, the formulations may include other agents conventional in the art having regard to the type of formulation in question, for example those suitable for oral administration may include flavouring agents.

The animal requiring treatment with a compound, salt or solvate of the present invention is usually a mammal, such as a human being.

The compounds of formula (I) or salts thereof, described above, are useful in therapy and in the preparation of medicaments for treating a disorder in a mammal, which is characterized by aberrant activity of at least one ErbB family PTK. In one embodiment of the present invention, the medicament prepared is useful in treating a disorder characterized by at least one ErbB family PTK, selected from EGFR, c-ErbB-2 and c-ErbB-4, exhibiting aberrant activity. In another embodiment, the medicament prepared is useful in treating a disorder characterized by at least two ErbB family PTKs, selected from EGFR, c-ErbB-2 and c-ErbB-4, exhibiting aberrant activity. In one embodiment of the use, the compounds of formula (I) or anhydrate or hydrate forms thereof, which are used to form the medicament, inhibit at least one ErbB family PTK, selected from EGFR, c-ErbB-2 and c-ErbB-4. In another embodiment of the use, the compounds of formula (I) or salts thereof, which are used to form the medicament, inhibit at least two ErbB family PTKs selected from EGFR, c-ErbB-2 and c-ErbB-4,

The disorders treated are as described above.

The compounds of the present invention and their salts and solvates, and physiologically functional derivatives thereof, may be employed alone or in combination with other therapeutic agents for the treatment of the above-mentioned conditions. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of formula (I), or a pharmaceutically acceptable salt or solvate thereof, or a physiologically functional derivative thereof, and the use of at least one other cancer treatment method. Preferably, combination therapies according to the present invention comprise the administration of at least one compound of formula (I), or a pharmaceutically acceptable salt or solvate thereof, or a physiologically functional derivative thereof, and at least one other pharmaceutically active agent, preferably an anti-neoplastic agent. The compounds of formula (I) or and the other pharmaceutically active agents may be administered together or separately and, when administered separately this may occur simultaneously or sequentially in any order. The amounts of the compounds of formula (I) and the other pharmaceutically active agents and the relative timings of administration will be selected in order to achieve the desired combined therapeutic effect.

The compounds of the Formula (I) or salts, solvates, or physiologically functional derivatives thereof and at least one additional cancer treatment therapy may be employed in combination concomitantly or sequentially in any therapeutically appropriate combination with such other anti-cancer therapies. In one embodiment, the other anti-cancer therapy is at least one additional chemotherapeutic therapy including administration of at least one anti-neoplastic agent. The administration in combination of a compound of formula (l), or salts, solvates, or physiologically functional derivatives thereof, ith other anti-neoplastic agents may be in combination in accordance with the invention by administration concomitantly in (1) a unitary pharmaceutical composition including both compounds or (2) separate pharmaceutical compositions each including one of the compounds. Altematively, the combination may be administered separately in a sequential manner wherein one anti-neoplastic agent is administered first and the other second or vice versa. Such sequential administration may be close in time or remote in time.

Anti-neoplastic agents may induce anti-neoplastic effects in a cell-cycle specific manner, i.e., are phase specific and act at a specific phase of the cell cycle, or bind DNA and act in a non cell-cycle specific manner, i.e., are non-cell cycle specific and operate by other mechanisms.

Anti-neoplastic agents useful in combination with the compounds and salts, solvates or physiologically functional derivatives thereof of formula I include the following:
(1) cell cycle specific anti-neoplastic agents including, but not limited to, diterpenoids such as paclitaxel and its analog docetaxel; vinca alkaloids such as vinblastine, vincristine, vindesine, and vinorelbine; epipodophyllotoxins such as etoposide and teniposide; fluoropyrimidines such as 5-fluorouracil and fluorodeoxyuridine; antimetabolites such as allopurinol, fludurabine, methotrexate, cladrabine, cytarabine, mercaptopurine and thioguanine; and camptothecins such as 9-amino camptothecin, irinotecan, CPT-11 and the various optical forms of 7-(4-methylpiperazino-methylene)-10,11-ethylenedioxy-20-camptothecin;
(2) cytotoxic chemotherapeutic agents including, but not limited to, alkylating agents such as melphalan, chlorambucil, cyclophosphamide, mechlorethamine, hexamethylmelamine, busulfan, carmustine, lomustine, and dacarbazine; anti-tumour antibiotics such as doxorubicin, daunomycin, epirubicin, idarubicin, mitomycin-C, dacttinomycin and mithramycin; and platinum coordination complexes such as cisplatin, carboplatin, and oxaliplatin; and
(3) other chemotherapeutic agents including, but not limited to, anti-estrogens such as tamoxifen, toremifene, raloxifene, droloxifene and iodoxyfene; progestrogens such as megestrol acetate; aromatase inhibitors such as anastrozole, letrazole, vorazole, and exemestane; antiandrogens such as flutamide, nilutamide, bicalutamide, and cyproterone acetate; LHRH agonists and antagagonists such as goserelin acetate and luprolide, testosterone 5α-dihydroreductase inhibitors such as finasteride; metalloproteinase inhibitors such as marimastat; antiprogestogens; urokinase plasminogen activator receptor function inhibitors; cyclooxygenase type 2 (COX-2) inhibitors such as celecoxib; other angiogenic inhibiting agents such as VEGFR inhibitors other than those described herein and TIE-2 inhibitors; growth factor function inhibitors such as inhibitors of the functions of hepatocyte growth factor; erb-B2, erb-B4, epidermal growth factor receptor (EGFR), platelet derived growth factor receptor (PDGFr), vascular endothelial growth factor receptor (VEGFR) other than those described in the present invention, and TIE-2; and other tyrosine kinase inhibitors such as cyclin dependent inhibitors such as CDK2 and CDK4 inhibitors.

Certain embodiments of the present invention will now be illustrated by way of example only. The following examples are intended for illustration only and are not intended to limit the scope of the invention in any way. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| i.v. (intravenous); | Hz (Hertz); |
| MHz (megahertz); | mol (moles); |
| mmol (millimoles); | rt (room temperature); |
| min (minutes); | h (hours); |
| mp (melting point); | TLC (thin layer chromatography); |
| Tᵣ (retention time); | RP (reverse phase); |
| MeOH (methanol); | i-PrOH (isopropanol); |
| TEA (triethylamine); | TFA (trifluoroacetic acid); |
| TFAA (trifluoroacetic anhydride); | THF (tetrahydrofuran); |
| DMSO (dimethylsulfoxide); | AcOEt (ethyl acetate); |
| DME (1,2-dimethoxyethane); | DCM (dichloromethane); |
| DCE (dichloroethane); | DMF (N,N-dimethylformamide); |
| DMPU (N,N′-dimethylpropyleneurea); | (CDI (1,1-carbonyldiimidazole); |
| IBCF (isobutyl chloroformate); | HOAc (acetic acid); |
| HOSu (N-hydroxysuccinimide); | HOBT (1-hydroxybenzotriazole); |
| mCPBA (meta-chloroperbenzoic acid; | EDC (ethylcarbodiimide hydrochloride); |
| BOC (tert-butyloxycarbonyl); | FMOC (9-fluorenylmethoxycarbonyl); |
| DCC (dicyclohexylcarbodiimide); | CBZ (benzyloxycarbonyl); |
| Ac (acetyl); | atm (atmosphere); |
| TMSE (2-(trimethylsilyl)ethyl); | TMS (trimethylsilyl); |
| TIPS (triisopropylsilyl); | TBS (t-butyldimethylsilyl); |
| DMAP (4-dimethylaminopyridine); | BSA (bovine serum albumin) |
| ATP (adenosine triphosphate); | HRP (horseradish peroxidase); |
| DMEM (Dulbecco's modified Eagle medium); | |
| HPLC (high pressure liquid chromatography); | |
| BOP (bis(2-oxo-3- | |
| oxazolidinyl)phosphinic chloride); | |
| TBAF (tetra-n-butylammonium fluoride); | |
| HBTU (O-Benzotriazole-1-yl- | |
| N,N,N′,N′-tetramethyluronium | |
| hexafluorophosphate). | |
| HEPES (4-(2-hydroxyethyl)-1- | |
| piperazine ethane sulfonic acid); | |
| DPPA (diphenylphosphoryl azide); | |
| fHNO₃ (fumed HNO₃); and | |
| EDTA (ethylenediaminetetraacetic acid). | |

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a JOEL JMS-AX505HA, JOEL SX-102, or a SCIEX-APliii spectrometer; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. All reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

Reported HPLC retention times (RT) were obtained on a Waters 2795 instrument attached to a Waters 996 diode array detector reading 210-500 nm. The column used was a Synergi Max-RP (50×2 mm) model #00B-4337-B0. Solvent gradient was 15% methanol:water to 100% methanol (0.1% formic acid) over 6 min. Flow rate was 0.8 mL/min. Injection volume was 3 microliters.

Chiral HPLC retention times for Examples 110 and 111 were obtained on a Berger Analytical SFC instrument attached to an HP 1100 diode array detector reading 280 nm. The column used was a Diacel ChiralCel-OJ (25×0.46 cm) model #OJH0CE-CF013. Eluting solvent was 30% methanol: 70% CO2 at 3000 psi and 40 C over 5 min. Flow rate was 2.0 mL/min. Injection volume was 10 microliters.

### Example 1

### Preparation of 3-[4-({3-Chloro-4-[(3-fluorobenzyl)oxy]phenyl}amino)thieno[3,2-d]pyrimidin-6-yl]prop-2-yn-1-ol hydrochloride

### Step A

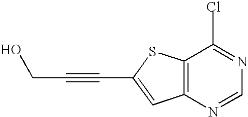

### 4-Chloro-thieno[3,2-d]pyrimidin-6-yl]prop-2-yn-1-ol

6-Bromo-4-chlorothieno[2,3-d]pyrimidine (Ref: M. J. Munchhof and S. B. Sobolov-Jaynes, Preparation of thienopyrimidines and thienopyridines as anticancer agents (PCT lnt. Appl. (1999), WO 9924440) (4.0 g, 16.0 mmol) was combined with propargyl alcohol (1.04 mL, 17.6 mmol), dichlorobis(triphenylphosphine) palladium (II) (0.32 g), copper (I) iodide (0.32 g, 1.7 mmol), and triethylamine (5.6 mL, 40.0 mmol) in 80 mL THF. The reaction mixture was heated to 60 C for 0.5 h, then cooled to room temperature and filtered through Celite. Silica gel was added to the filtrate and the solvent was removed in vacuo. The resulting solid was loaded on to a column of silica gel and eluted with 10-50% ethyl acetate in hexane gradient to give 2.4 g intermediate 4-chloro-thieno[3,2-d]pyrimidin-6-yl]prop-2-yn-1-ol. APCl MS: 225 (MH⁺), HPLC RT: 3.19 min.

### Step B

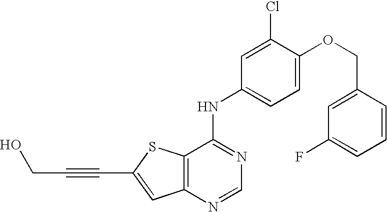

### 3-[4-({3-Chloro-4-[(3-fluorobenzyl)oxy]phenyl}amino)thieno[3,2-d]pyrimidin-6-yl]prop-2-yn-1-ol hydrochloride

4-chloro-thieno[3,2-d]pyrimidin-6-yl]prop-2-yn-1-ol (2.39 g, 10.6 mmol) was combined with known 3-chloro-4-[(3-fluorobenzyl)oxy]aniline (G. S. Cockerill and K. E. Lackey, Preparation of anilinoquinazolines as protein tyrosine kinase inhibitors. PCT Appl. (2001), WO0104111) (2.68 g, 10.6 mmol) in 50 mL isopropyl alcohol. The mixture was heated to 60 C for 16 h. Tan solid precipitated and the reaction mixture was cooled to room temperature. The solid was filtered, rinsed with 10 mL ethyl acetate, and dried in vacuo to give 4.4 g of the title compound as the HCl salt. ¹H NMR (DMSO-d₆) δ 10.77 (br s, 1H), 8.76 (s,1H), 7.87 (d, 1H), 7.65 (s, 1H), 7.58 (dd, 1H), 7.50-7.43 (m, 1H). 7.33-7.28 (m, 3H), 7.21-7.15 (m, 1H), 5.27 (s, 2H), 4.40 (s, 2H), —OH signal not observed due to broadening. HPLC RT: 3.77 min. HRMS: 553.1591 (MH⁺).

### Example 2

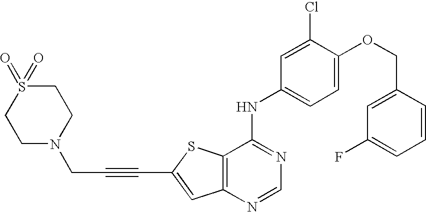

### N-{3-Chloro-4-[(3-fluorobenzyl)oxy]phenyl}-6-[3-(1,1-dioxidothiomorpholin-4-yl)prop-1-ynyl]thieno[3,2-d]pyrimidin-4-amine hydrochloride

The title compound was prepared as the HCl salt from 6-bromo-4-chlorotliieno[2,3-d]pyrimidine by a procedure analogous to example 1 using commercially available 4-(2-propynyl)-thiomorpholine 1,1-dioxide and known 3-chloro-4-[(3-fluorobenzyl)oxy]aniline. HPLC RT: 3.76 min. HRMS: 557.0876 (MH⁺).

### Example 3

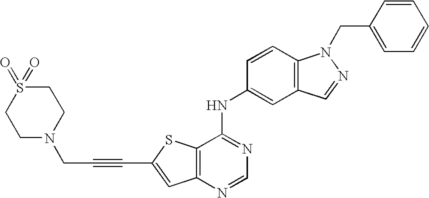

### N-(1-Benzyl-1H-indazol-5-yl)6-[3-(1,1-dioxidothiomorpholin-4-yl)prop-1-ynyl]thieno[3,2-d]pyrimidin4-amine hydrochloride

The title compound was prepared as the HCl salt from 6-bromo-4-chlorothieno[2,3d]pyrimidine by a procedure analogous to example 1 using commercially available 4-(2-propynyl)-thiomorpholine 1,1-dioxide and known 5-amino-1-benzyl-indazole (G. S. Cockerill, K. E. Lackey, Preparation of quinazolinylamines and analogs as protein tyrosine kinase inhibitors. PCT Appl. 1999, WO9935132). HPLC RT: 3.13 min. HRMS: 529.1496 (MH⁺).

### Example 4

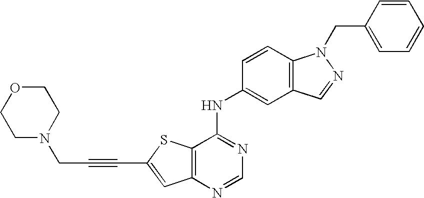

### N-(1-Benzyl-1H-indazol-5-yl)-6-(3-morpholin-4-ylprop-1-ynyl)thieno[3,2-d]pyrimidin-4-amine hydrochloride

The title compound was prepared as the HCl salt from 6-bromo-4-chlorothieno[2,3-d]pyrimidine by a procedure analogous to example 1 using commercially available 4-(2-propynyl)morpholine and known 5-amino-1-benzyl-indazole, HPLC RT: 2.82 min. HRMS: 481.1795 (MH⁺).

### Example 5

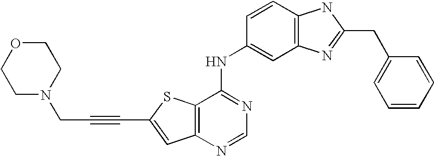

### N-(2-Benzyl-1H-benzimidazol-5-yl)6-(3-morpholin-4-ylprop-1-ynyl)thieno[3,2-d]pyrimidin4-amine

The title compound was prepared from 6-bromo4-chlorothieno[2,3-d]pyrimidine by a procedure analogous to example 1 using commercially available 4-(2-propynyl)-morpholine and known 5-amino-2-benzyl-1H-benzimidazole (G. S. Cockerill, K. E. Lackey, Preparation of quinazolinylamines and analogs as protein tyrosine kinase inhibitors. PCT Appl. (1999), WO9935132), HPLC RT: 1.91 min. HRMS: 481.1803 (MH⁺).

### Example 6

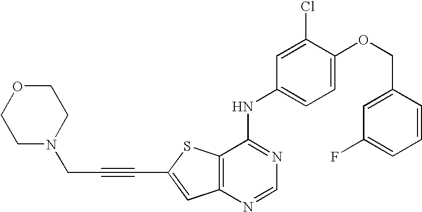

### N-{3-Chloro-4-[(3-fluorobenzyl)oxy]phenyl}-6-(3-morpholin-4-ylprop-1-ynyl)thieno[3,2-d]pyrimidin-4-amine hydrochloride

The title compound was prepared as the HCl salt from 6-bromo4-chlorothieno[2,3-d]pyrimidine by a procedure analogous to example 1 using commercially available 4-(2-propynyl)-morpholine and known 3-chloro-4-[(3-fluorobenzyl)oxy]aniline. HPLC RT: 3.54 min. HRMS: 509.1209 (MH⁺).

### Example 7

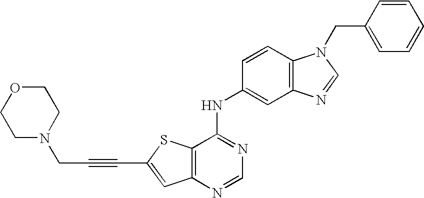

### N-(1-Benzyl-1H-benzimidazol-5-yl)6-(3-morpholin-4-ylprop-1-ynyl)thieno[3,2-d]pyrimidin-4-amine hydrochloride

The title compound was prepared as the HCl salt from 6-bromo4-chlorothieno[2,3-d]pyrimidine by a procedure analogous to example 1 using commercially available 4-(2-propynyl)-morpholine and known 5-amino-1-benzyl-benzimidazole (G. S. Cockerill, et al., Preparation of heterocyclyl-substituted quinazolines as protein tyrosine kinase inhibitors. PCT Appl. (1997) WO9703069). HPLC RT: 2.17 min. HRMS: 481.1821 (MH⁺).

### Example 8

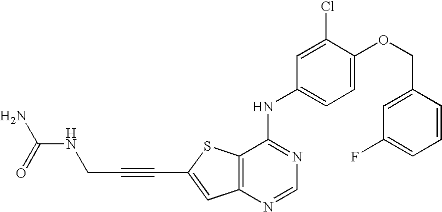

### N-{3-[4-({3-Chloro-4-[(3-fluorobenzyl)oxy]phenyl}amino)thieno[3,2-d]pyrimidin-6-yl]prop-2-ynyl}urea hydrochloride

The title compound was prepared as the HCl salt from 6-bromo-4-chlorothieno[2,3-d]pyrimidine by a procedure analogous to example 1 using commercially available 2-propynylurea and known 3-chloro-4-[(3-fluorobenzyl)oxy]aniline, ¹H NMR (DMSO-d₆) δ 10.73 (br s, 1H), 8.76 (s, 1H), 7.86 (d, 1H), 7.62 (s, 1H), 7.57 (dd, 1H), 7.49-7.44 (m, 1H), 7.33-7.28 (m, 3H), 7.21-7.16 (m, 1H), 6.50 (br s, 1H), 5.50 (br s, 2H), 5.28 (s, 2H), 4.13 (s, 2H). HPLC RT: 3.59 min. HRMS: 482.0860 (MH⁺).

### Example 9

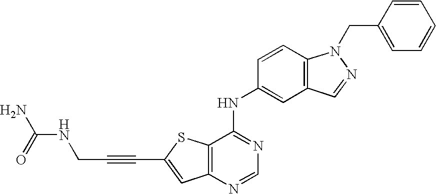

### N-(3-{4-[(1-Benzyl-1H-indazol-5-yl)amino]thieno[3,2-d]pyrimidin-6-yl}prop-2-ynyl)urea hydrochloride

The title compound was prepared as the HCl salt from 6-bromo-4-chlorothieno[2,3-d]pyrimidine by a procedure analogous to example 1 using commercially available 2-propynylurea and known 5-amino-1-benzyl-indazole, HPLC RT: 2.85 min. HRMS: 454.1458 (MH⁺).

### Example 10

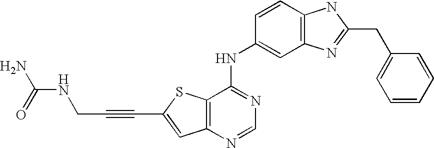

### N-(3-{4-[(2-Benzyl-1H-benzimidazol-5-yl)amino]thieno[3,2-d]pyrimidin-6-yl}prop-2-ynyl)urea hydrochloride

The title compound was prepared as the HCl salt from 6-bromo-4-chlorothieno[2,3-d]pyrimidine by a procedure analogous to example 1 using commercially available 2-propynylurea and known 5-amino-2-benzyl-1H-benzimidazole. HPLC RT: 2.02 min. HRMS: 454.1445 (MH⁺).

### Example 11

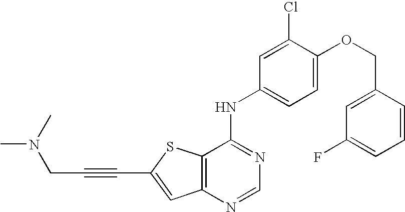

### N-{3-Chloro-4-[(3-fluorobenzyl)oxy]phenyl}-6-[3-(dimethylamino)prop-1-ynyl]thieno[3,2-d]pyrimidin-4-amine hydrochloride

The title compound was prepared as the HCl salt from 6-bromo-4-chlorothieno[2,3-d]pyrimidine by a procedure analogous to example 1 using commercially available N,N-dimethyl-2-propyn-1-amine and known 3-chloro-4-[(3-fluorobenzyl)oxy]aniline. HPLC RT: 2.85 min. HRMS: 467.1126 (MH⁺).

### Example 12

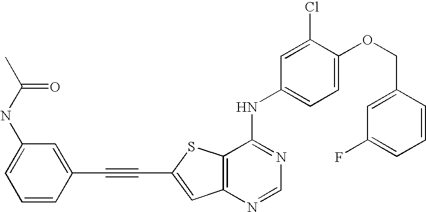

### N-(3-{[4-({3-Chloro-4-[(3-fluorobenzyl)oxy]phenyl}amino)thieno[3,2-d]pyrimidin-6-yl]ethynyl}phenyl)acetamide hydrochloride

The title compound was prepared as the HCl salt from 6-bromo-4-chlorothieno[2,3-d]pyrimidine by a procedure analogous to example 1 using commercially available N-(3-ethynylphenyl)-acetamide and known 3-chloro-4-[(3-fluorobenzyl)oxy]aniline. HPLC RT: 4.18 min. HRMS: 543.1057 (MH⁺).

### Example 13

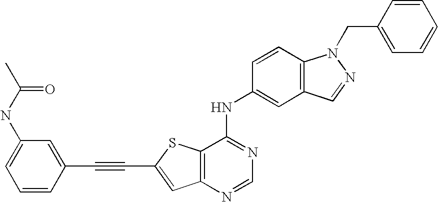

### N-[3-({4-[(1-Benzyl-1H-indazol-5-yl)amino]thieno[3,2-d]pyrimidin-6-yl}ethynyl)phenyl]acetamide hydrochloride

The title compound was prepared as the HCl salt from 6-bromo4-chlorothieno[2,3-d]pyrimidine by a procedure analogous to example 1 using commercially available N-(3-ethynylphenyl)-acetamide and known 5-amino-1-benzyl-indazole. HPLC RT: 3.70 min. HRMS: 515.1664 (MH⁺).

### Example 14

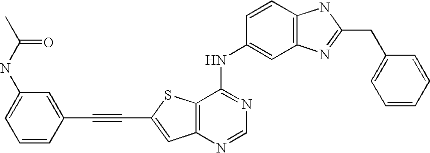

### N-[3-({4-[(2-Benzyl-1H-benzimidazol-5-yl)amino]thieno[3,2-d]pyrimidin-6-yl}ethynyl)phenyl]acetamide hydrochloride

The title compound was prepared as the HCl salt from 6-bromo-4-chlorothieno[2,3-d]pyrimidine by a procedure analogous to example 1 using commercially available N-(3-ethynylphenyl)-acetamide and known 5-amino-2-benzyl-1H-benzimidazole. HPLC RT: 2.94 min. HRMS: 515.1643 (MH⁺).

### Example 15

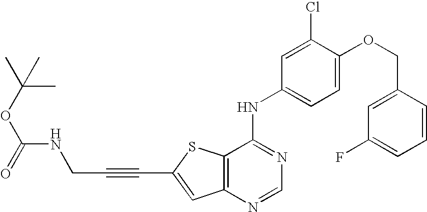

### tert-Butyl 3-[4-({3-chloro-4-[(3-fluorobenzyl)oxy]pheny}amino)thieno[3,2-d]pyrimidin-6-yl]prop-2-ynylcarbamate hydrochloride

The title compound was prepared as the HCl salt from 6-bromo-4-chlorothieno[2,3-d]pyrimidine by a procedure analogous to example 1 using known N-(tert-butoxycarbonyl)propargylamine (N. Pitt, et al.,Tetrahedron Lett. (1999), 40, 3811-3814.) and known 3-chloro-4-[(3-fluorobenzyl)oxy]aniline. HPLC RT: 4.16 min. HRMS: 539.1321 (MH⁺).

### Example 16

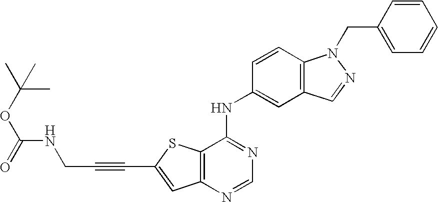

### tert-Butyl 3-{4-[(1-benzyl-1H-indazol-5-yl)amino]thieno[3,2-d]pyrimidin-6-yl}prop-2-ynylcarbamate hydrochloride

The title compound was prepared as the HCl salt from 6-bromo-4-chlorothieno[2,3-d]pyrimidine by a procedure analogous to example 1 using known N-(tert-butoxycarbonyl)propargylamine and known 5-amino-1-benzyl-indazole, HPLC RT: 3.67 min. HRMS: 511.1900 (MH⁺).

### Example 17

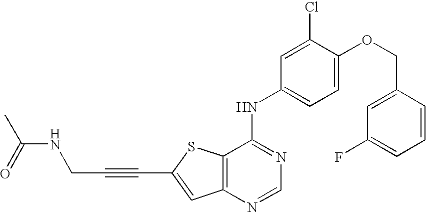

### N-{3-[4-({3-Chloro-4-[(3-fluorobenzyl)oxy]phenyl}amino)thieno[3,2-d]pyrimidin-6-yl]prop-2-ynyl}acetamide hydrochloride

The title compound was prepared as the HCl salt from 6-bromo-4-chlorothieno[2,3-d]pyrimidine by a procedure analogous to example 1 using known N-2-propynyl-acetamide (A. De Meijere, et al., Synthesis (1991) 547-60) and known 3-chloro-4-[(3-fluorobenzyl)oxy]aniline., HPLC RT: 3.74 min. HRMS: 481.0905 (MH⁺).

### Example 18

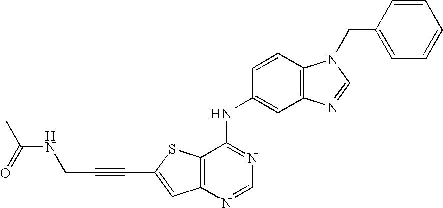

### N-(3-{4-[(1-Benzyl-1H-indazol-5-yl)amino]thieno[3,2-d]pyrimidin-6-yl}prop-2-ynyl)acetamide hydrochloride

The title compound was prepared as the HCl salt from 6-bromo-4-chlorothieno[2,3-d]pyrimidine by a procedure analogous to example 1 using known N-2-propynyl-acetamide. and known 5-amino-1-benzyl-indazole, HPLC RT: 3.04 min. HRMS: 453.1487 (MH⁺).

### Example 19

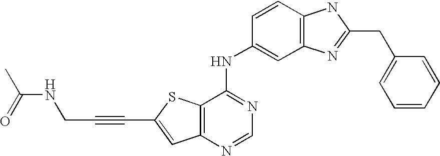

### N-(3-{4-[(2-Benzyl-1H-benzimidazol-5-yl)amino]thieno[3,2-d]pyrimidin-6-yl)prop-2-ynyl)acetamide hydrochloride

Title compound was prepared as the HCl salt from 6-bromo-4-chlorothieno[2,3-d]pyrimidine by a procedure analogous to example 1 using known N-2-propynyl-acetamide and known 5-amino-2-benzyl-1H-benzimidazole. HPLC RT: 2.19 min. HRMS: 453.1501 (MH⁺).

### Example 20

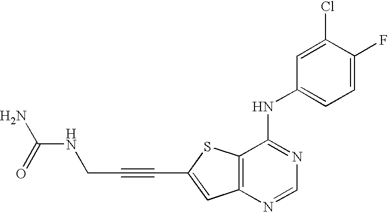

### N-(3-{4-[(3-Chloro-4-fluorophenyl)amino]thieno[3,2-d]pyrimidin-6-yl}prop-2-ynyl)urea hydrochloride

Title compound was prepared as the HCl salt from 6-bromo-4-chlorothieno[2,3-d]pyrimidine by a procedure analogous to example 1 using commercially available 2-propynylurea and commercially available 3-chloro-4-fluoroaniline, HPLC RT: 3.19 min. HRMS: 376.0432 (MH⁺).

### Example 21

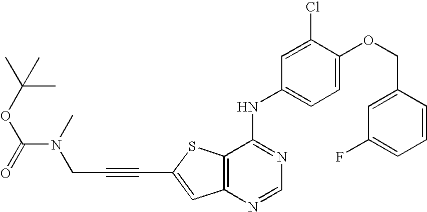

### tert-butyl 3-[4-({3-chloro-4-[(3-fluorobenzyl)oxy]phenyl}amino)thieno[3,2-d]pyrimidin-6-yl]prop-2-ynyl(methyl)carbamate

The title compound was prepared as the HCl salt from 6-bromo4-chlorothieno[2,3-d]pyrimidine by a procedure analogous to example 1 using known N-methyl-N-(tert-butoxycarbonyl)propargylamine (B. J. Bradbury, et al., J. Med. Chem. (1990) 33, 741-8.) and known 3-chloro-4-[(3-fluorobenzyl)oxy]aniline. HPLC RT: 4.34 min. HRMS: 553.1482 (MH⁺).

### Example 22

### N-(1-Benzyl-1H-indol-5-yl)6-ethynylthieno[3,2-d]pyrimidin-4-amine hydrochloride

### Step A

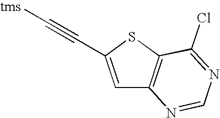

### 6-(Trimethylsilyl)ethynyl-4-chloro-thieno[3,2-d]pyrimidine

6-Bromo-4-chlorothieno[2,3-d]pyrimidine (0.80 g, 3.2 mmol) was combined with trimethylsilyl acetylene (0.54 mL, 3.8 mmol), dichlorobis(triphenylphosphine) palladium (II) (0.11 g), copper (I) iodide (0.06 g, 0.32 mmol), and triethylamine (0.90 mL, 6.4 mmol) in 20 mL THF. The reaction mixture was heated to 60 C for 0.5 h, then cooled to room temperature and filtered through Celite and concentrated in vacuo. The resulting crude was loaded on to a column of silica gel and eluted with 5-10% ethyl acetate in hexane gradient to give 0.44 of 6-(trimethylsilyl)ethynyl-4-chloro-thieno[3,2-d]pyrimidine. APCl MS: 267 (MH⁺) HPLC RT: 4.56 min.

### Step B

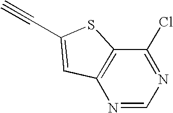

### 6-(Ethynyl-4-chloro-thieno[3,2-d]pyrimidine

6-(Trimethylsilyl)ethynyl-4-chloro-thieno[3,2-d]pyrimidine (0.44 g, 1.6 mmol) was dissolved in 20 mL THF and cooled in an ice bath. A 1.0M solution of tetrabutylammonium fluoride in THF (1.8 mL, 1.8 mmol) was added and the reaction was stirred 5 min. The reaction mixture was poured into 75 mL water and extracted with ethyl acetate (2×50 mL). The extracts were washed with brine (50 mL), dried over sodium sulfate, filtered and concentrated. The resulting crude was loaded on to a column of silica gel and eluted with 10-25% ethyl acetate in hexane gradient to give 0.19 g of 6-ethynyl-4-chloro-thieno[3,2-d]pyrimidine. APCl MS: 195 (MH⁺) HPLC RT: 3.28 min.

### Step C

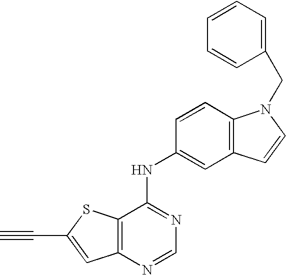

### N-(1-Benzyl-1H-indol-5-yl)-6-ethynylthieno[3,2-d]pyrimidin-4-amine hydrochloride

6-Ethynyl-4-chloro-thieno[3,2-d]pyrimidine (0.020 g, 0.103 mmol) was combined with known 5-amino-1-benzylindole (G. S. Cockerill, et al., Preparation of heterocyclyl-substituted quinazolines as protein tyrosine kinase inhibitors. PCT Appl. (1997) WO9703069) (0.023 g, 0.103 mmol) in 1.5 mL isopropyl alcohol. The mixture was heated to 60 C for 16 h. The reaction mixture was cooled to room temperature. The solid was filtered, rinsed with 2 mL ethyl acetate, and dried in vacuo to give 0.011 g of product as the HCl salt. ¹H NMR (DMSO-d₆) δ 11.00 (br s, 1H), 8.74 (s, 1H), 7.74 (s, 1H), 7.65 (s, 1H), 7.62 (d, 1H), 7.54 (d, 1H), 7.33-7.17 (m, 6H), 6.57 (d, 1H), 5.49 (s, 2H), 5.14 (s, 1H). HPLC RT: 3.50 min. HRMS: 381.1184 (MH⁺).

### Example 23

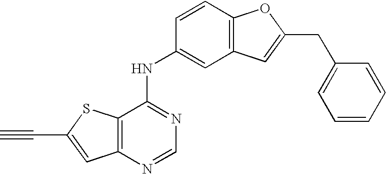

### N-(2-Benzyl-1-benzofuran-5-yl)6-ethynylthieno[3,2-d]pyrimidin-4-amine hydrochloride

The title compound was prepared as the HCl salt from 6-bromo-4-chlorothieno[2,3-d]pyrimidine and known 5-amino-2-(benzyl)benzofuran (S. Jegham, et al., Compounds derived from 3-(benzofuran-5-yl)oxazolidin-2-one, their preparation, and their therapeutic use as MAO inhibitors. PCT Appl. (1997) WO9717346) by a procedure analogous to example 22. HPLC RT: 3.92 min. HRMS: 382.1022 (MH⁺).

### Example 24

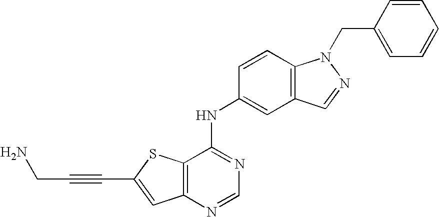

### 6-(3-Aminoprop-1-ynyl)-N-(1-benzyl-1H-indazol-5-yl)thieno[3,2-d]pyrimidin-4-amine

tert-Butyl-3-{4-[(1-benzyl-1H-indazol-5-yl)amino]thieno[3,2-d]pyrimidin-6-yl}prop-2-ynylcarbamate hydrochloride (example 16) (0.011 g, 0.020 mmol) was covered with chloroform (1 mL) and trifluoroacetic acid (0.2 mL) was added. The reaction mixture was stirred 1 h. The reaction mixture was poured into saturated sodium bicarbonate (10 mL), extracted with chloroform (2×20 mL), dried over sodium sulfate, filtered and concentrated in vacuo to give 0.006 g of product. HPLC RT: 2.16 min. HRMS: 411.1399 (MH⁺).

### Example 25

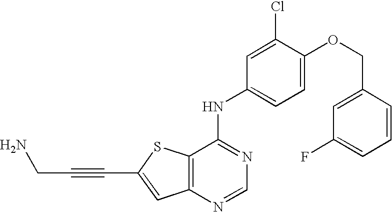

### 6-(3-Aminoprop-1-ynyl)-N-{3-chloro-4-[(3-fluorobenzyl)oxy]phenyl}thieno[3,2-d]pyrimidin-4-amine

Title compound was prepared from tert-butyl-3-[4-({3-chloro-4-[(3-fluorobenzyl)oxy]phenyl}amino)thieno[3,2-d]pyrimidin-6-yl]prop-2-ynylcarbamate (example 15) by a procedure analogous to example 24. ¹H NMR (CDCl₃) δ 8.63 (s, 1H), 8.09 (s, 1H), 7.87 (s, 1H), 7.39-7.22 (m, 9H), 5.65 (s, 2H), 3.66 (s, 2H), —NH₂ signal not observed due to broadening. HPLC RT: 2.80 min. HRMS: 439.0799 (MH⁺).

### Example 26

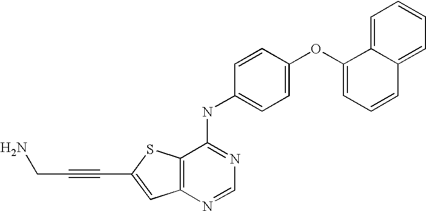

### 6-(3-Aminoprop-1-ynyl)-N-[4-(1-naphthyloxy)phenyl]thieno[3,2-d]pyrimidin-4-amine

tert-Butyl-3-(4-chlorothieno[3,2-d]pyrimidin-6-yl)prop-2-ynylcarbamate prepared by a procedure analogous to example 1 was combined with 4-(1-naphthyloxy)aniline (J. Schuhmacher, et al., (1998) DE19740785) in isopropyl alcohol. The mixture was heated to 90° C. for 16 h. Three drops concentrated HCl were added and the mixture was subsequently heated at 50° C. for 1-3 h. The reaction cooled, filtered and treated with aqueous saturated NaHCO₃ and then partitioned into ethyl acetate. The ethyl acetate was then dried over sodium sulfate, filtered, and concentrated to dryness to give a brown solid. ¹H NMR (300 MHz, DMSO-d₆) δ 4.14 (m, 2H), 7.00 (d, 1H), 7.11 (d, 2H), 7.49 (m, 1H), 7.58 (m, 2H), 7.72 (m, 4H), 8.00 (d,1H), 8.11 (d, 1H), 8.42 (bs, 2H), 8.61 (s, 1H), 10.04 (s, 1H). LC-MS (ES+) m/z 423 (M⁺+H); HPLC RT: 2.98 min.

### Example 27

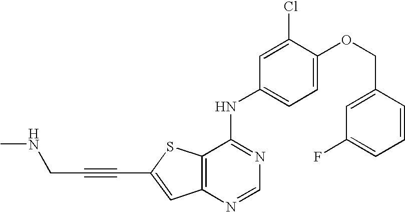

### N-{3-chloro-4-[(3-fluorobenzyl)oxy]phenyl}-6-[3-(methylamino)prop-1-ynyl]thieno[3,2-d]pyrimidin-4-amine

Title compound was prepared from ted-butyl 3-[4-({3-chloro-4-[(3-fluorobenzyl)oxy]phenyl}amino)thieno[3,2-d]pyrimidin-6-yl]prop-2-ynyl(methyl)carbamate by a procedure analogous to example 24. ¹H NMR (CDCl₃) δ 8.65 (s, 1H), 7.62 (d, 1H), 7.42 (s, 1H), 7.58 (dd, 1H), 7.39-7.33 (m, 2H), 7.26-7.22 (m, 2H), 7.06-7.02 (m, 1H), 6.99 (d, 1H), 6.88 (br s, 1H),5.20 (s, 2H), 6.67 (s, 2H), 2.54 (s, 3H). HPLC RT: 2.83 min. HRMS: 453.0955 (MH⁺).

### Example 28

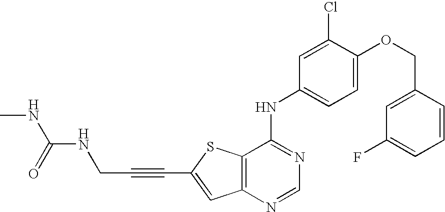

### N-{3-[4-({3-Chloro-4-[(3-fluorobenzyl)oxy]phenyl}amino)thieno[3,2-d]pyrimidin-6-yl]prop-2-ynyl}-N′-methylurea

6-(3-Aminoprop-1-ynyl)-N-(1-benzyl-1H-indazol-5-yl)thieno[3,2-d]pyrimidin4-amine (example 25) (0.040 g, 0.091 mmol) was combined with methyl isocyanate (0.005 mL, 0.101 mmol) in chloroform (2 mL) and stirred for 1 h. The reaction mixture was poured into water (25 mL), extracted with ethyl acetate (2×25 mL), washed with brine (25 mL), dried over sodium sulfate, filtered and concentrated in vacuo to give 0.030 g of product. ¹H NMR (DMSO-d₆) δ 9.80 (s, 1H), 8.56 (s, 1H), 7.92 (d, 1H), 7.61 (dd, 1H), 7.57 (s, 1H), 7.50-7.42 (m, 1H), 7.32-7.15 (m, 4H), 6.48 (br t, 1H), 6.02 (br q, 1H), 5.24 (s, 2H), 4.13 (d, 2H), 2.56 (d, 3H). HPLC RT: 3.66 min. HRMS: 496.1028 (MH⁺).

### Example 29

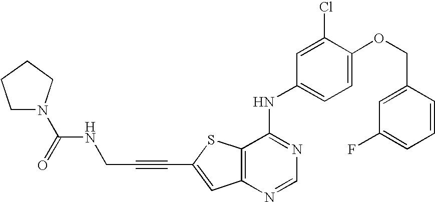

### N-{3-[4-({3-Chloro-4-[(3-fluorobenzyl)oxy]pheny}amino)thieno[3,2-d]pyrimidin-6-yl]prop-2-ynyl}-N′-cyclopentylurea

The title compound was prepared from 6-(3-aminoprop-1-ynyl)-N-(1-benzyl-1H-indazol-5-yl)thieno[3,2-d]pyrimidin-4-amine and commercially available cyclopentyl isocyanate by a procedure analogous to example 28. HPLC RT: 4.02 min. HRMS: 550.1481 (MH⁺).

### Example 30

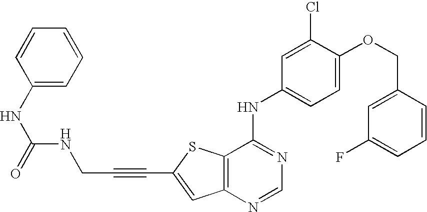

### N-{3-[4-({3-Chloro-4-[(3-fluorobenzyl)oxy]phenyl}amino)thieno[3,2d]pyrimidin-6-yl]prop-2-ynyl)N′-phenylurea

The title compound was prepared from 6-(3-aminoprop-1-ynyl)-N-(1-benzyl-1H-indazol-5-yl)thieno[3,2-d]pyrimidin-4-amine (example 25) and commercially available phenyl isocyanate by a procedure analogous to example 28. HPLC RT: 4.01 min. HRMS: 558.1155 (MH⁺).

### Example 31

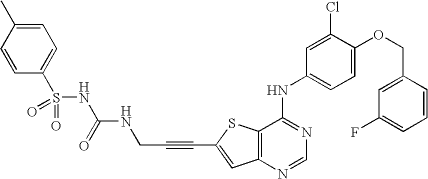

### N-[({3-[4-({3-Chloro-4-[(3-fluorobenzyl)oxy]phenyl}amino)thieno[3,2-d]pyrimidin-6-yl]prop-2-ynyl}amino)carbonyl]-4-methylbenzenesulfonamide

The title compound was prepared from 6-(3-aminoprop-1-ynyl)-N-(1-benzyl-1H-indazol-5-yl)thieno[3,2-d]pyrimidin-4-amine (example 25) and commercially available tosyl isocyanate by a procedure analogous to example 28. HPLC RT: 3.98 min. HRMS: 636.0943 (MH⁺).

### Example 32

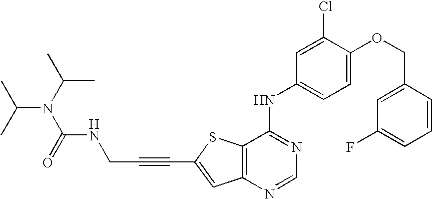

### N′-{3-[4-({3-Chloro-4-[(3-fluorobenzyl)oxy]phenyl}amino)thieno[3,2-d]pyrimidin-6-yl]prop-2-ynyl}-N,N-diisopropylurea

6-(3-aminoprop-1-ynyl)-N-(1-benzyl-1H-indazol-5-yl)thieno[3,2-d]pyrimidin-4-amine (0.040 g, 0.091 mmol) (example 25) was combined with 1,1′-carbonyl diimidazole (0.015 g, 0.091 mmol) in dimethylacetamide (1 mL) and stirred for 0.25 h. Diisopropylamine (0.038 mL, 0.270 mmol) was added and the reaction stirred 16 h. The reaction mixture was poured into water (25 mL), extracted with ethyl acetate (2×25 mL), washed with brine (25 mL), dried over sodium sulfate, filtered and concentrated in vacuo. Column chromatography (5% methanol in dichloromethane as eluent) gave 0.031 g of product. ¹H NMR (CDCl₃) δ 8.63 (s, 1H), 7.61 (d, 1H), 7.42 (s, 1H), 7.40-7.33 (m, 2H), 7.25-7.21 (m, 2H), 7.05-7.01 (m, 1H), 6.98 (d, 1H), 5.19 (s, 2H), 4.45 (t, 1H), 4.33 (d, 2H), 3.85 (septet, 2H), 1.26 (d, 12H), —NH signal not observed due to broadening. HPLC RT: 4.14 min. HRMS: 566.1784 (MH⁺).

### Example 33

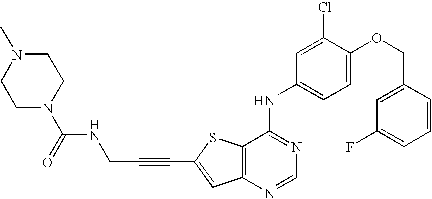

### N-{3-[4-({3-Chloro-4-[(3-fluorobenzyl)oxy]phenyl}amino)thieno[3,2-d]pydmidin-6-yl]prop-2-ynyl}-4-methylpiperazine-1-carboxamide

The title compound was prepared from 6-(3-aminoprop-1-ynyl)-N-(1-benzyl-1H-indazol-5-yl)thieno[3,2-d]pyrimidin-4-amine (example 25) and N-methylpiperazine by a procedure analogous to example 32. HPLC RT: 2.85 min. HRMS: 565.1569 (MH⁺).

### Example34

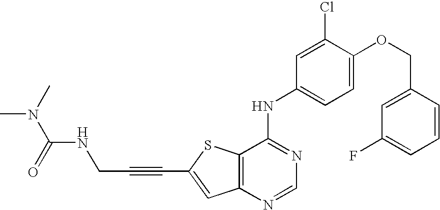

### N′-{3-[4-({3-Chloro-4-[(3-fluorobenzyl)oxy]phenyl}amino)thieno[3,2-d]pyrimidin-6-yl]prop-2-ynyl}-N,N-dimethylurea

The title compound was prepared from 6-(3-aminoprop-1-ynyl)-N-(1-benzyl-1H-indazol-5-yl)thieno[3,2-d]pyrimidin-4-amine (example 25) and dimethylamine by a procedure analogous to example 32. HPLC RT: 3.74 min. HRMS: 510.1176 (MH⁺).

### Example 35

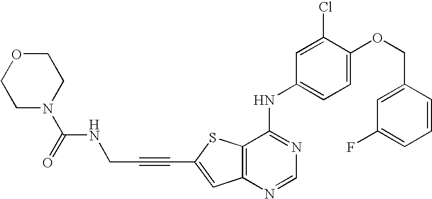

### N-{3-[4-({3-Chloro-4-[(3-fluorobenzyl)oxy]phenyl}amino)thieno[3,2-d]pyrimidin-6-yl]prop-2-ynyl}morpholine-4-carboxamide

The title compound was prepared from 6-(3-aminoprop-1-ynyl)-N-(1-benzyl-1H-indazol-5-yl)thieno[3,2-d]pyrimidin-4-amine (example 25) and 2-(methylsulfonyl)ethylamine by a procedure analogous to example 32. HPLC RT: 3.74 min. HRMS: 552.1278 (MH⁺).

### Example 36

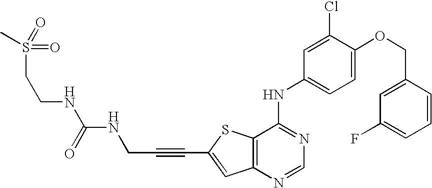

### N-{3-[4-({3-Chloro-4-[(3-fluorobenzyl)oxy]phenyl}amino)thieno[3,2-d]pyrimidin-6-yl]prop-2-ynyl)-N′-[2-(methylsulfonyl)ethyl]urea

The title compound was prepared from 6-(3-aminoprop-1-ynyl)-N-(1-benzyl-1H-indazol-5-yl)thieno[3,2-d]pyrimidin-4-amine and known 2-(methylsulfonyl)ethylamine (G. S. Cockerill, et al., Preparation of anilinoquinazolines as protein tyrosine kinase inhibitors. PCT Appl. (2001) WO0104111) by a procedure analogous to example 32. HPLC RT: 3.58 min. HRMS: 588.0933 (MH⁺).

### Example 37

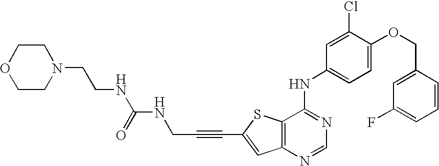

### N-{3-[4-({3-Chloro-4-[(3-fluorobenzyl)oxy]phenyl}amino)thieno[3,2-d]pyrimidin-6-yl]prop-2-ynyl}-N′-(2-morpholin-4-ylethyl)urea

The title compound was prepared from 6-(3-aminoprop-1-ynyl)-N-(1-benzyl-1H-indazol-5-yl)thieno[3,2-d]pyrimidin-4-amine (example 25) and commercially available N-(2-aminoethyl)morpholine by a procedure analogous to example 32. HPLC RT: 2.89 min. HRMS: 595.1681 (MH⁺).

### Example 38

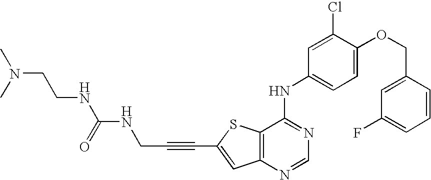

### N-{3-[4-({3-Chloro-4-[(3-fluorobenzyl)oxy]phenyl}amino)thieno[3,2-d]pyrimidin-6-yl]prop-2-ynyl}-N′-[2-(dimethylamino)ethyl]urea

The title compound was prepared from 6-(3-aminoprop-1-ynyl)-N-(1-benzyl-1H-indazol-5-yl)thieno[3,2-d]pyrimidin-4-amine (example 25) and commercially available 2-(N,N-dimethylamino)ethylamine by a procedure analogous to example 32. HPLC RT: 2.84 min. HRMS: 553.1591 (MH⁺).

### Example 39

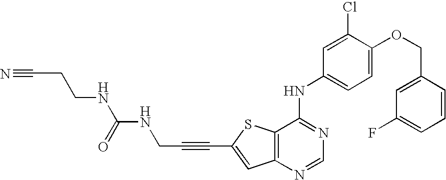

### N-{3-[4-({3-Chloro-4-[(3-fluorobenzyl)oxy]phenyl}amino)thieno[3,2-d]pyrimidin-6-yl]prop-2-ynyl}-N′-(2-cyanoethyl)urea

The title compound was prepared from 6-(3-aminoprop-1-ynyl)-N-(1-benzyl-1H-indazol-5-yl)thieno[3,2-d]pyrimidin-4-amine (example 25) and commercially available 2-cyanoethylamine by a procedure analogous to example 32. HPLC RT: 3.64 min. HRMS: 535.1118 (MH⁺).

### Example 40

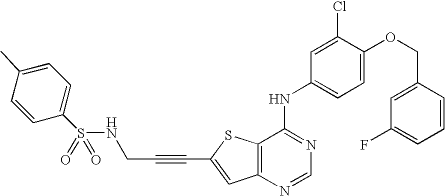

### N-{3-[4-({3-Chloro-4-[(3-fluorobenzyl)oxy]phenyl}amino)thieno[3,2-d]pyrimidin-6-yl]prop-2-ynyl}-4-methylbenzenesulfonamide

6-(3-aminoprop-1-ynyl)-N-(1-benzyl-1H-indazol-5-yl)thieno[3,2-d]pyrimidin-4-amine (0.040 g, 0.091 mmol) (example 25) was combined with tosyl chloride (0.019 g, 0.100 mmol) in chloroform (3 mL). Triethylamine (0.025 mL, 0.182 mmol) was added and the reaction mixture was stirred for 2.5 h. The reaction mixture was poured into water (25 mL), extracted with ethyl acetate (2×25 mL), washed with brine (25 mL), dried over sodium sulfate, filtered and concentrated in vacuo. Column chromatography (ethyl acetate as eluent) gave 0.048 g of product. ¹H NMR (DMSO-d₆) δ 9.93 (br s, 1H), 8.60 (s, 1H), 8.24-8.20 (m, 1H) 7.89 (d, 1H), 7.74 (d, 2H), 7.58 (dd, 1H), 7.50-7.08 (m, 8H), 5.25 (s, 2H), 4.08 (d, 2H), 2.29 (s, 3H). HPLC RT: 4.06 min. HRMS: 593.0893 (MH⁺).

### Example 42

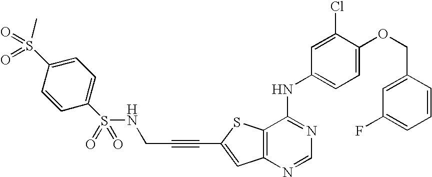

### N-[3-[4-({3-Chloro-4-[(3-fluorobenzyl)oxy]phenyl}amino)thieno[3,2-d]pyrimidin-6-yl]prop-2-ynyl}-4-(methylsulfonyl)benzenesulfonamide

The title compound was prepared from 6-(3-aminoprop-1-ynyl)-N-(1-benzyl-1H-indazol-5-yl)thieno[3,2-d]pyrimidin-4-amine (example 25) and commercially available 4-(methylsulfonyl)benzenesulfonyl chloride by a procedure analogous to example 40. HPLC RT: 3.77 min. HRMS: 657.0500 (MH⁺).

### Example 42

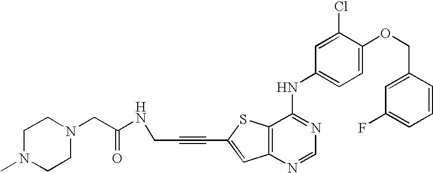

### N-{3-[4-({3-Chloro-4-[(3-fluorobenzyl)oxy]phenyl}amino)thieno[3,2-d]pyrimidin-6-yl]prop-2-ynyl}-2-(4-methylpiperazin-1-yl)acetamide

To a mixture of 6-(3-aminoprop-1-ynyl)-N-{3-chloro-4-[(3-fluorobenzyl)oxy]phenyl}thieno[3,2-d]pyrimidin-4-amine (example 25) (50 mg, 0.114 mmol), known 4-methylpiperazine acetic acid (J. Rautio, et al., J. Med. Chem. (2000) 43, 1489-1494) (27 mg, 0.117 mmol) and triethylamine (32 mg, 0.32 mmol) in DMF (3 mL) was added diethyl cyanophosphonate (30 mg, 0.19 mmol). The reaction mixture was stirred at room temperature and monitored by LCMS until all starting material was consumed. The reaction was quenched with brine (10 mL) and the resulting solid filtered off and washed well with water to afford 30 mg of the product. ¹H NMR (d₆-DMSO) δ 9.72 (s, 1H), 8.55 (s, 1H), 8.26 (t, 1H), 7.89 (d, 1H), 7.56-7.59 (m, 2H), 7.42-7.48 (m, 1H), 7.29 (t, 2H), 7.23 (d, 1H), 7.16 (d, 1H), 5.23 (s, 2H), 4.21 (d, 2H), 2.94 (s, 2H), 2.20-2.45 (m, 8H), 2.14 (s, 3H). MS (ES): 579 (MH⁺), 581 (MH⁺).

### Example 43

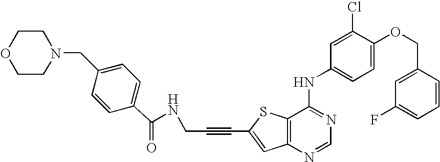

### N-{3-[4-({3-chloro-4-[(3-fluorobenzyl)oxy]phenyl}amino)thieno[3,2-d]pyrimidin-6-yl]prop-2-ynyl}-4-(morpholin-4-ylmethyl)benzamide

The title compound was prepared from 6-(3-aminoprop-1-ynyl)-N-(1-benzyl-1H-indazol-5-yl)thieno[3,2-d]pyrimidin-4-amine (example 25) and commercially available 4-(morpholinomethyl)benzoic acid by a procedure analogous to example 42. HPLC RT: 3.12 min. HRMS: 642.1742 (MH⁺).

### Example 44

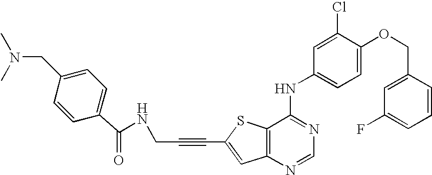

### N-{3-[4-({3-chloro-4-[(3-fluorobenzyl)oxy]phenyl}amino)thieno[3,2-d]pyrimidin-6-yl]prop-2-ynyl}-4-[(dimethylamino)methyl]benzamide

The title compound was prepared from 6-(3-aminoprop-1-ynyl)-N-(1-benzyl-1H-indazol-5-yl)thieno[3,2-d]pyrimidin-4-amine (example 25) and known 4-[(dimethylamino)methyl]benzoic acid (H. G. Kazmirowski, et al, Pharmazie (1967)22, 465-70) by a procedure analogous to example 42. HPLC RT: 3.03 min. HRMS: 600.1636 (MH⁺).

### Example 45

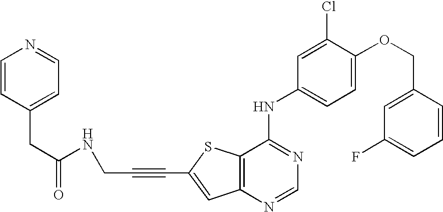

### N-{3-[4-({3-chloro-4-[(3-fluorobenzyl)oxy]phenyl}amino)thieno[3,2-d]pyrimidin-6-yl]prop-2-ynyl)-2-pyridin-4-ylacetamide

The title compound was prepared from 6-(3-aminoprop-1-ynyl)-N-(1-benzyl-1H-indazol-5-yl)thieno[3,2-d]pyrimidin-4-amine and commercially available 4-pyridine-acetic acid by a procedure analogous to example 42. HPLC RT: 3.42 min. HRMS: 558.1167 (MH⁺).

### Example 46

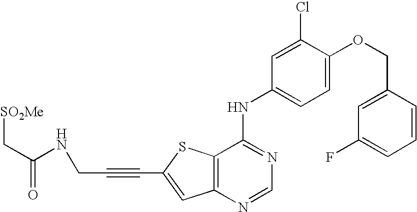

### N-{3-[4-({3-chloro-4-[(3-fluorobenzyl)oxy]phenyl}amino)thieno[3,2-d]pyrimidin-6-yl]prop-2-ynyl}-2-(methylsulfonyl)acetamide

The title compound was prepared from 6-(3-aminoprop-1-ynyl)-N-(1-benzyl-1H-indazol-5-yl)thieno[3,2-d]pyrimidin-4-amine (example 25) and commercially available methanesulfonylacetic acid by a procedure analogous to example 42. HPLC RT: 3.77 min. HRMS: 559.0677 (MH⁺).

### Example 47

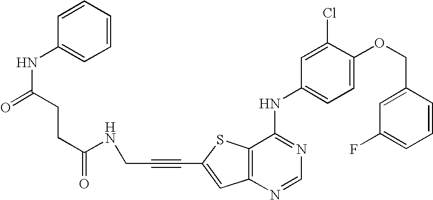

### N˜1˜-(3-[4-({3-chloro-4-[(3-fluorobenzyl)oxy]phenyl}amino)thieno[3,2-d]pyrimidin-6-yl]prop-2-ynyl}-N˜4˜-phenylsuccinamide

The title compound was prepared from 6-(3-aminoprop-1-ynyl)-N-(1-benzyl-1H-indazol-5-yl)thieno[3,2-d]pyrimidin-4-amine (example 25) and commercially available succinanilic acid by a procedure analogous to example 42. HPLC RT: 4.13 min. HRMS: 614.1429 (MH⁺).

### Example 48

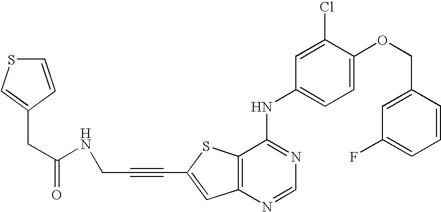

### N-{3-[4-({3-chloro-4-[(3-fluorobenzyl)oxy]phenyl}amino)thieno[3,2-d]pyrimidin-6-yl]prop-2-ynyl}-2-thien-3-ylacetamide

The title compound was prepared from 6-(3-aminoprop-1-ynyl)-N-(1-benzyl-1H-indazol-5-yl)thieno[3,2-d]pyrimidin-4-amine and commercially available 3-thiophene-acetic acid by a procedure analogous to example 42. HPLC RT: 4.12 min. HRMS: 563.0778 (MH⁺).

### Example 49

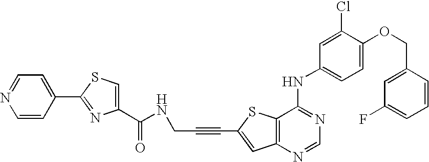

### N-{3-[4-({3-chloro-4-[(3-fluorobenzyl)oxy]phenyl}amino)thieno[3,2-d]pyrimidin-6-yl]prop-2-ynyl}-2-pyridin-4-yl-1,3-thiazole-4-carboxamide

The title compound was prepared from 6-(3-aminoprop-1-ynyl)-N-(1-benzyl-1H-indazol-5-yl)thieno[3,2-d]pyrimidin-4-amine (example 25) and commercially available 2-(pyrid-4-yl)thiazole-4-carboxylic acid by a procedure analogous to example 42. HPLC RT: 4.11 min. HRMS: 627.0840 (MH⁺).

### Example 50

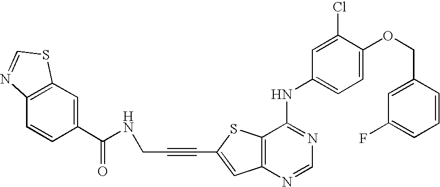

### N-{3-[4-({3-chloro-4-[(3-fluorobenzyl)oxy]phenyl}amino)thieno[3,2-d]pyrimidin-6-yl]prop-2-ynyl}-1,3-benzothiazole-6-carboxamide

The title compound was prepared from 6-(3-aminopmp-1-ynyl)-N-(1-benzyl-1H-indazol-5-yl)thieno[3,2-d]pyrimidin-4-amine and commercially available benzothiazole-6-carboxylic acid by a procedure analogous to example 42. HPLC RT: 4.07 min. HRMS: 600.0731 (MH⁺).

### Example 51

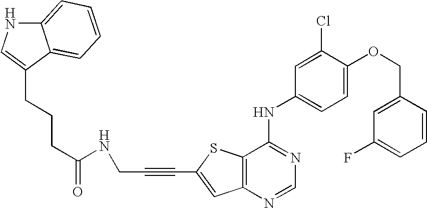

### N-{3-[4-({3-chloro-4-[(3-fluorobenzyl)oxy]phenyl}amino)thieno[3,2-d]pyrimidin-6-yl]prop-2-ynyl}-4-(1H-indol-3-yl)butanamide

The title compound was prepared from 6-(3-aminoprop-1-ynyl)-N-(1-benzyl-1H-indazol-5-yl)thieno[3,2-d]pyrimidin-4-amine and commercially available 3-indolebutyric acid by a procedure analogous to example 42. HPLC RT: 4.18 min. HRMS: 624.1636 (MH⁺).

### Example 52

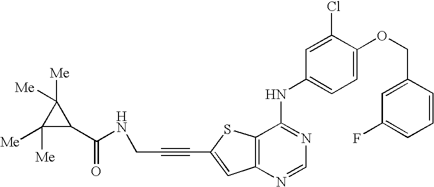

### N-{3-[4-(}3-chloro-4-[(3-fluorobenzyl)oxy]phenyl}amino)thieno[3,2-d]pyrimidin-6-yl]prop-2-ynyl}-2,2,3,3-tetramethylcyclopropanecarboxamide

The title compound was prepared from 6-(3-aminoprop-1-ynyl)-N-(1-benzyl-1H-indazol-5-yl)thieno[3,2-d]pyrimidin-4-amine and commercially available 2,23,3-tetramethylcyclopropane carboxylic acid by a procedure analogous to example 42. HPLC RT: 4.40 min. HRMS: 563.1684 (MH⁺).

### Example 53

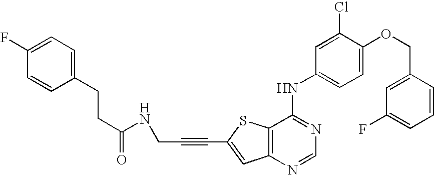

### N-{3-[4-({3-chloro-4-[(3-fluorobenzyl)oxy]phenyl}amino)thieno[3,2-d]pyrimidin-6-yl]prop-2-ynyl}-3-(4-fluorophenyl)propanamide

The title compound was prepared from 6-(3-aminoprop-1-ynyl)-N-(1-benzyl-1H-indazol-5-yl)thieno[3,2-d]pyrimidin-4-amine and commercially available 3-(4-fluorophenyl)propionic acid by a procedure analogous to example 42. HPLC RT: 4.25 min. HRMS: 589.1277 (MH⁺).

### Example 54

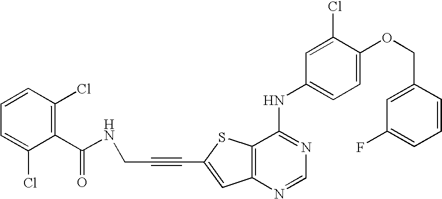

### 2,6-dichloro-N-{3-[4-({3-chloro-4-[(3-fluorobenzyl)oxy]phenyl}amino)thieno[3,2-d]pyrimidin-6-yl]prop-2-ynyl}benzamide

The title compound was prepared from 6-(3-aminoprop-1-ynyl)-N-(1-benzyl-1H-indazol-5-yl)thieno[3,2-d]pyrimidin-4-amine and commercially available 2,6-dichlorobenzoic acid by a procedure analogous to example 42. HPLC RT: 4.14 min. HRMS: 611.0278 (MH⁺).

### Example 55

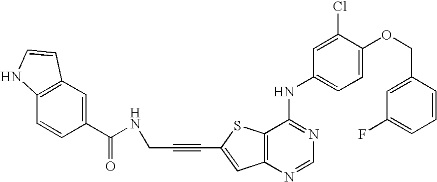

### N-{3-[4-({3-chloro-4-[(3-fluorobenzyl)oxy]phenyl}amino)thieno[3,2-d]pyrimidin-6-yl]prop-2-ynyl}-1H-indole-5-carboxamide

The title compound was prepared from 6-(3-aminoprop-1-ynyl)-N-(1-benzyl-1H-indazol-5-yl)thieno[3,2-d]pyrimidin-4-amine and commercially available indole-5-carboxylic acid by a procedure analogous to example 42. HPLC RT: 4.01 min. HRMS: 582.1167 (MH⁺).

### Example 56

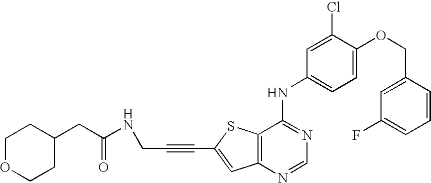

### N-{3-[4-({3-chloro-4-[(3-fluorobenzyl)oxy]phenyl}amino)thieno[3,2-d]pyrimidin-6-yl]prop-2-ynyl}-2-tetrahydro-2H-pyran-4-ylacetamide

The title compound was prepared from 6-(3-aminoprop-1-ynyl)-N-(1-benzyl-1H-indazol-5-yl)thieno[3,2-d]pyrimidin-4-amine and commercially available tetrahydropyranyl-4-acetic acid by a procedure analogous to example 42. HPLC RT: 3.98 min. HRMS: 565.1476 (MH⁺).

### Example 57

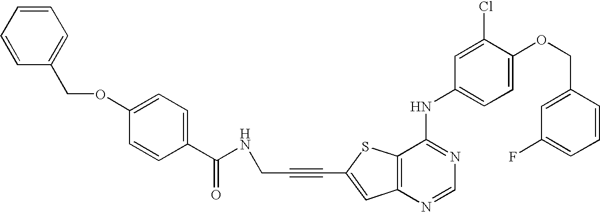

### 4-(benzyloxy)-N-{3-[4-({3-chloro-4-[(3-fluorobenzyl)oxy]phenyl}amino)thieno[3,2-d]pyrimidin-6-yl]prop-2-ynyl}benzamide

The title compound was prepared from 6-(3-aminoprop-1-ynyl)-N-(1-benzyl-1H-indazol-5-yl)thieno[3,2-d]pyrimidin-4-amine and commercially available 4-benzyloxybenzoic acid by a procedure analogous to example 42. HPLC RT: 4.56 min. HRMS: 649.1476 (MH⁺).

### Example 58

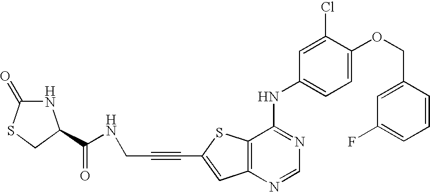

### (4R)—N-{3-[4-({3-chloro-4-[(3-fluorobenzyl)oxy]phenyl}amino)thieno[3,2-d]pyrimidin-6-yl]prop-2-ynyl}-2-oxo-1,3-thiazolidine-4-carboxamide

The title compound was prepared from 6-(3-aminoprop-1-ynyl)-N-(1-benzyl-1H-indazol-5-yl)thieno[3,2-d]pyrimidin-4-amine and commercially available (−) 2-oxo4-thiazolidine carboxylic acid by a procedure analogous to example 42. HPLC RT: 3.84 min. HRMS: 568.0680 (MH⁺).

### Example 59

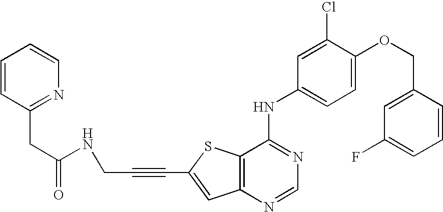

### N-{3-[4-({3-chloro-4-[(3-fluorobenzyl)oxy]phenyl}amino)thieno[3,2-d]pyrimidin-6-yl]prop-2-ynyl}-2-pyridin-2-ylacetamide

The title compound was prepared from 6-(3-aminoprop-1-ynyl)-N-(1-benzyl-1H-indazol-5-yl)thieno[3,2-d]pyrimidin-4-amine and commercially available 2-pyridylacetic acid by a procedure analogous to example 42. HPLC RT: 3.78 min. HRMS: 558.1167 (MH⁺).

### Example 60

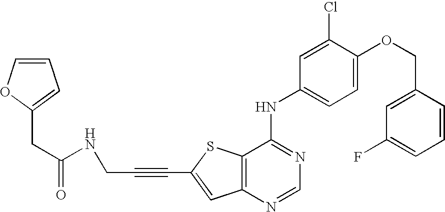

### N-{3-[4-({3-chloro-4-[(3-fluorobenzyl)oxy]phenyl}amino)thieno[3,2-d]pyrimidin-6-yl]prop-2-ynyl}-2-(2-furyl)acetamide

The title compound was prepared from 6-(3-aminoprop-1-ynyl)-N-(1-benzyl-1H-indazol-5-yl)thieno[3,2-d]pyrimidin-4-amine and commercially available 2-furylacetic acid by a procedure analogous to example 42. HPLC RT: 4.02 min. HRMS: 547.1007 (MH⁺).

### Example 61

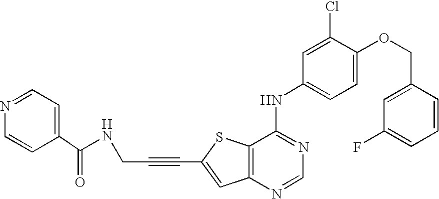

### N-{3-[4-({3-chloro-4-[(3-fluorobenzyl)oxy]phenyl}amino)thieno[3,2-d]pyrimidin-6-yl]prop-2-ynyl}isonicotinamide

The title compound was prepared from 6-(3-aminoprop-1-ynyl)-N-(1-benzyl-1H-indazol-5-yl)thieno[3,2-d]pyrimidin-4-amine and commercially available isonicotinic acid by a procedure analogous to example 42. HPLC RT: 3.95 min. HRMS: 544.1010 (MH⁺).

### Example 62

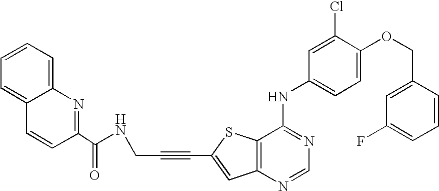

### N-{3-[4-({3-chloro-4-[(3-fluorobenzyl)oxy]phenyl}amino)thieno[3,2-d]pyrimidin-6-yl]prop-2-ynyl}quinoline-2-carboxamide

The title compound was prepared from 6-(3-aminoprop-1-ynyl)-N-(1-benzyl-1H-indazol-5-yl)thieno[3,2-d]pyrimidin-4-amine and commercially available quinaldic acid by a procedure analogous to example 42. HPLC RT: 4.43 min. HRMS: 594.1167 (MH⁺).

### Example 63

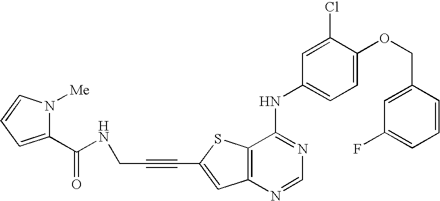

### N-{3-[4-({3-chloro-4-[(3-fluorobenzyl)oxy]phenyl}amino)thieno[3,2-d]pyrimidin-6-yl]prop-2-ynyl}-1-methyl-1H-pyrrole-2-carboxamide

The title compound was prepared from 6-(3-aminoprop-1-ynyl)-N-(1-benzyl-1H-indazol-5-yl)thieno[3,2-d]pyrimidin-4-amine and commercially available 1-methyl-2-pyrrole carboxylic acid by a procedure analogous to example 42. HPLC RT: 4.10 min. HRMS: 546.1167 (MH⁺).

### Example 64

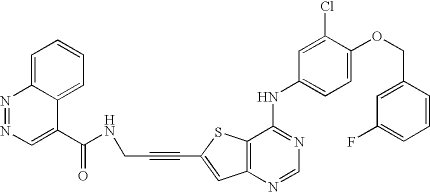

### N-{3-[4-({3-chloro-4-[(3-fluorobenzyl)oxy]phenyl}amino)thieno[3,2-d]pyrimidin-6-yl]prop-2-ynyl}cinnoline-4-carboxamide

The title compound was prepared from 6-(3-aminoprop-1-ynyl)-N-(1-benzyl-1H-indazol-5-yl)thieno[3,2-d]pyrimidin-4-amine and commercially available cinnoline 4-carboxylic acid by a procedure analogous to example 42. HPLC RT: 4.08 min. HRMS: 595.1119 (MH⁺).

### Example 65

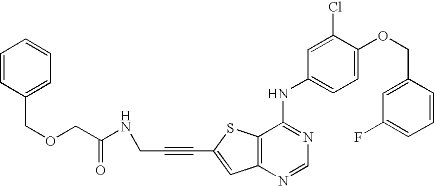

### 2-(benzyloxy)-N-{3-[4-({3-chloro-4-[(3-fluorobenzyl)oxy]phenyl}amino)thieno[3,2-d]pyrimidin-6-yl]prop-2-ynyl}acetamide

The title compound was prepared from 6-(3-aminoprop-1-ynyl)-N-(1-benzyl-1H-indazol-5-yl)thieno[3,2-d]pyrimidin-4-amine and commercially available benzyloxyacetic acid by a procedure analogous to example 42. HPLC RT: 4.26 min. HRMS: 587.1320 (MH⁺).

### Example 66

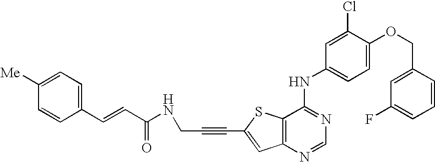

### (2E)-N-{3-[4-({3-chloro-4-[(3-fluorobenzyl)oxy]phenyl}amino)thieno[3,2-d]pyrimidin-6-yl]prop-2-ynyl}-3-(4-methylphenyl)prop-2-enamide

The title compound was prepared from 6-(3-aminoprop-1-ynyl)-N-(1-benzyl-1H-indazol-5-yl)thieno[3,2-d]pyrimidin-4-amine and commercially available 4-methylcinnamic acid by a procedure analogous to example 42. HPLC RT: 4.38 min. HRMS: 583.1371 (MH⁺).

### Example 67

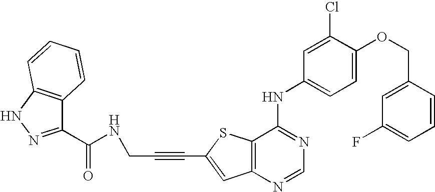

### N-{3-[4-({3-chloro-4-[(3-fluorobenzyl)oxy]phenyl}amino)thieno[3,2-d]pyrimidin-6-yl]prop-2-ynyl}-1H-indazole-3-carboxamide

The title compound was prepared from 6-(3-aminoprop-1-ynyl)-N-(1-benzyl-1H-indazol-5-yl)thieno[3,2-d]pyrimidin-4-amine and commercially available indazole-3-carboxylic acid by a procedure analogous to example 42. HPLC RT: 4.17 min. HRMS: 583.1119 (MH⁺).

### Example 68

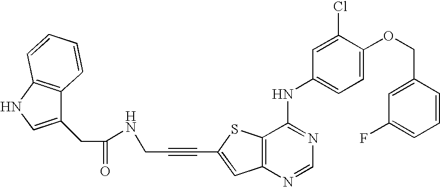

### N-{3-[4-({3-chloro-4-[(3-fluorobenzyl)oxy]phenyl}amino)thieno[3,2-d]pyrimidin-6-yl]prop-2-ynyl}-2-(1H-indol-3-yl)acetamide

The title compound was prepared from 6-(3-aminoprop-1-ynyl)-N-(1-benzyl-1H-indazol-5-yl)thieno[3,2-d]pyrimidin-4-amine and commercially available indole-3-acetic acid by a procedure analogous to example 42. HPLC RT: 4.12 min. HRMS: 596.1323 (MH⁺).

### Example 69

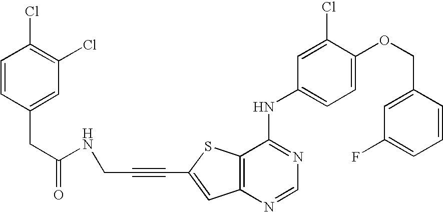

### N-{3-[4-({3-chloro-4-[(3-fluorobenzyl)oxy]phenyl}amino)thieno[3,2-d]pyrimidin-6-yl]prop-2-ynyl}-2-(3,4-dichlorophenyl)acetamide

Title compound was prepared from 6-(3-aminoprop-1-ynyl)-N-(1-benzyl-1H-indazol-5-yl)thieno[3,2-d]pyrimidin-4-amine and commercially available 3,4-dichlorophenyl acetic acid by a procedure analogous to example 42. HPLC RT: 4.38 min. HRMS: 625.0435 (MH⁺).

### Example 70

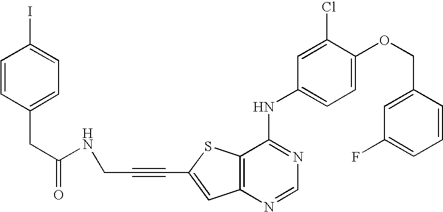

### N-{3-[4-({3-chloro-4-[(3-fluorobenzyl)oxy]phenyl}amino)thieno[3,2-d]pyrimidin-6-yl]prop-2-ynyl}-2-(4-iodophenyl)acetamide

The title compound was prepared from 6-(3-aminoprop-1-ynyl)-N-(1-benzyl-1H-indazol-5-yl)thieno[3,2-d]pyrimidin-4-amine and commercially available 4-iodophenyl acetic acid by a procedure analogous to example 42. HPLC RT: 4.36 min. HRMS: 683.0181 (MH⁺).

### Example 71

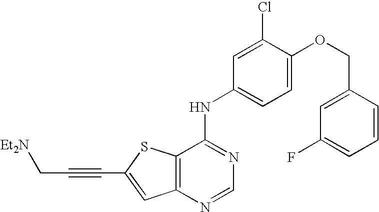

### N-{3-chloro-4-[(3-fluorobenzyl)oxy]phenyl}-6-[3-(diethylamino)prop-1-ynyl]thieno[3,2-d]pyrimidin-4-amine

To a mixture of 6-(3-aminoprop-1-ynyl)-N-{3-chloro-4-[(3-fluorobenzyl)oxy]phenyl}thieno[3,2-d]pyrimidin-4-amine (50 mg, 0.114 mmol) and acetaldehyde (45 mg, 1.02 mmol) in dichloroethane (25 mL) was added acetic acid (38 mg, 0.63 mmol) followed by sodium triacetoxyborohydride (92 mg, 0.43 mmol). The reaction mixture was stirred at room temperature and monitored by LCMS and further additions of sodium triacetoxyborohydride were made until all 6-(3-aminoprop-1-ynyl)-N-{3-chloro-4-[(3-fluorobenzyl)oxy]phenyl}thieno[3,2-d]pyrimidin-4-amine was consumed. The reaction was quenched with saturated sodium bicarbonate solution (20 mL) and the organic phase separated, dried over magnesium sulfate and concentrated. The crude oil was subjected to column chromatography on silica gel (AcOEt) to afford the title compound in 22 mg yield. ¹H NMR (d₆-DMSO) δ 9.68 (s, 1H), 8.55 (s, 1H), 7.92 (d, 1H), 7.59 (dd, 1H), 7.56 (s, 1H), 7.44 (q, 1H), 7.14-7.31 (m, 4H), 5.23 (s, 2H), 3.70 (s, 2H), 2.53 (d, 4H), 1.01 (t, 6H). HRMS: 495.1421 (MH⁺).

### Example 72

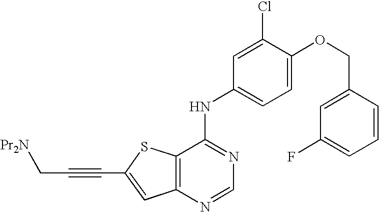

### N-{3-chloro-4-[(3-fluorobenzyl)oxy]phenyl}-6-[3-(dipropylamino)prop-1-ynyl]thieno[3,2-d]pyrimidin-4-amine

Title compound was prepared from 6-(3-aminoprop-1-ynyl)-N-(1-benzyl-1H-indazol-5-yl)thieno[3,2-d]pyrimidin-4-amine and commercially available propioaldehyde by a procedure analogous to example 71. HPLC RT: 3.23 min. HRMS: 523.1735 (MH⁺).

### Example 73

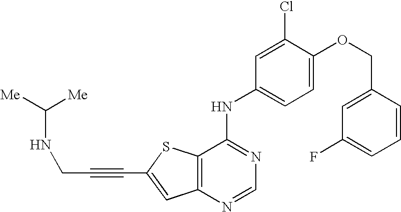

### N-{3-chloro-4-[(3-fluorobenzyl)oxy]phenyl}-6-[3-(isopropylamino)prop-1-ynyl]thieno[3,2-d]pyrimidin-4-amine

The title compound was prepared from 6-(3-aminoprop-1-ynyl)-N-(1-benzyl-1H-indazol-5-yl)thieno[3,2-d]pyrimidin-4-amine and acetone by a procedure analogous to example 71. HPLC RT: 2.92 min. HRMS: 481.1265 (MH⁺).

### Example 74

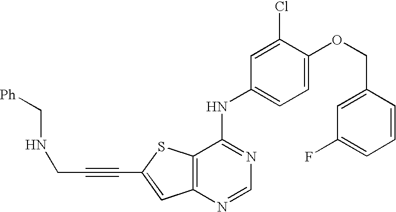

### 6-[3-(benzylamino)prop-1-ynyl]-N-{3-chloro-4-[(3-fluorobenzyl)oxy]phenyl}thieno[3,2-d]pyrimidin-4-amine

The title compound was prepared from 6-(3-aminoprop-1-ynyl)-N-(1-benzyl-1H-indazol-5-yl)thieno[3,2-d]pyrimidin-4-amine and benzaldehyde by a procedure analogous to example 71. HPLC RT: 3.15 min. HRMS: 529.1265 (MH⁺).

### Example 75

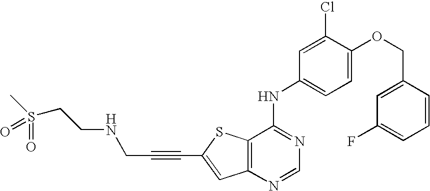

### N-{3-Chloro-4-[(3-fluorobenzyl)oxy]phenyl}-6-(3-{[2-(methylsulfonyl)ethyl]amino}prop-1-ynyl)thieno[3,2-d]pyrimidin-4-amine hydrochloride

6-(3-Aminoprop-1-ynyl)-N-(1-benzyl-1H-indazol-5-yl)thieno[3,2-d]pyrimidin4-amine (example 25) (0.050 g, 0.114 mmol) was combined with methyl vinyl sulfone (0.020 mL, 0.228 mmol) in isopropyl alcohol (3 mL) and heated to 120° C. in a sealed tube for 3 h. The reaction mixture was cooled to r.t. and poured into water (30 mL). The mixture was extracted with ethyl acetate (2×30 mL), washed with brine (20 mL), dried over sodium sulfate, filtered and concentrated in vacuo. Column chromatography (5-10% methanol in dichloromethane gradient) followed by acidification with HCl (4.0M in dioxane) gave 0.017 g of product as the HCl salt. HPLC RT: 3.05 min. HRMS: 545.0892 (MH⁺).

### Example 76

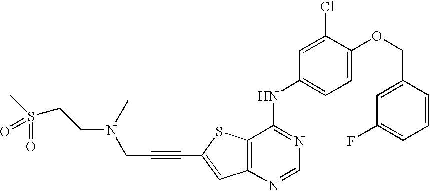

### N-{3-chloro-4-[(3-fluorobenzyl)oxy]phenyl}-6-(3-{methyl[2-(methylsulfonyl)ethyl]amino}prop-1-ynyl)thieno[3,2-d]pyrimidin-4-amine

Title compound was prepared from N-{3-chloro-4-[(3-fluorobenzyl)oxy]phenyl}-6-[3-(methylamino)prop-1-ynyl]thieno[3,2-d]pyrimidin-4-amine by a procedure analogous to that shown in example 75. ¹H NMR (CDCl₃) δ 8.65 (s, 1H), 7.62 (d, 1H), 7.44 (s, 1H), 7.39-7.34 (m, 2H), 7.26-7.21 (m, 2H), 7.08-7.00 (m, 2H), 6.99 (d, 1H), 5.20 (s, 2H), 3.65 (s, 2H), 3.17 (t, 2H), 3.03 (t, 2H), 3.02 (s, 3H), 2.42 (s, 3H). HPLC RT: 3.67 min. HRMS: 559.1036 (MH⁺).

### Example 77

### Preparation of N-{3-chloro-4-[(3-fluorobenzyl)oxy]phenyl}-6-(pyrimidin-2-ylethynyl)thieno[3,2-d]pyrimidin-4-amine

### Step A

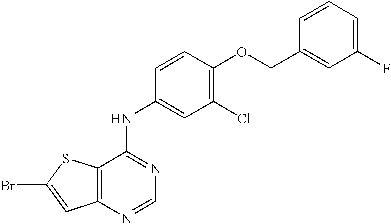

### 6-Bromo-N-{3-chloro-4-[(3-fluorobenzyl)oxy]phenyl}thieno[3,2-d]pyrimidin-4-amine hydrochloride

6-Bromo-4-chlorothieno[3,2-d]pyrimidine (2) (1.05 g, 4 mmol) and 3-chloro-4-[(3-fluorobenzyl)oxy]aniline (986 mg, 3.9 mmol) were heated at 60° C. for 3 h in isopropanol (30 mL). The mixture was concentrated and the resulting material was triturated with ethyl ether and collected by suction filtration to yield the product (1.7 g) as a white solid.

### Step B

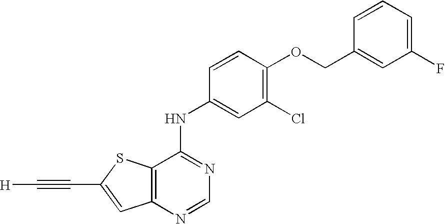

### N-{3-chloro-4-[(3-fluorobenzyl)oxy]phenyl}-6-ethynylthieno[3,2-d]pyrimidin4-amine

6-Bromo-N-{3-chloro-4-[(3-fluorobenzyl)oxy]phenyl}thieno[3,2-d]pyrimidin-4-amine hydrochloride (1.0 g, 2.0 mmol) was combined with Cul (45 mg, 0.24 mmol), dichlorobis(triphenylphosphine)palladium(II) (57 mg, 0.08 mmol), THF (14 mL), triethylamine (0.74 mL, 5.3 mmol), and trimethylsilylacetylene (0.37 mL, 2.62 mmol). The mixture stirred at room temperature for 6 h, concentrated and purified by silica gel chromatography (eluting with 3:1 to 2:1 hexane/ethyl acetate). The resulting silyl acetylene intermediate (618 mg) was dissolved in THF (17 mL) and cooled to 0° C. A 1.0 M solution of TBAF in THF (1.4 mL, 1.4 mmol) was added and the mixture was stirred 1 h. The reaction was partitioned between ethyl acetate and water, the organic layer was separated and dried (Na₂SO₄) filtered and concentrated. The resulting solid was purified by silica gel chromatography (eluting with 7:3 to 6:4 hexane/ethyl acetate) to give the title compound (400 mg) as an orange solid.

ESI MS (positive ion): (M-H) 410.2 ¹H NMR (300 MHz, DMSO) 8 5.03 (s, 1H), 5.25 (s, 2H), 7.14-7.21 (m, 1H), 7.23-7.22 (m, 3H), 7.43-7.50 (d, 1H), 7.61 (dd, J=8.9, 2.6Hz, 1H), 7.71 (s, 1H), 7.92 (d, J=2.5 Hz, 1H), 8.59 (s, 1H), 9.78 (s, 1H).

### Step C

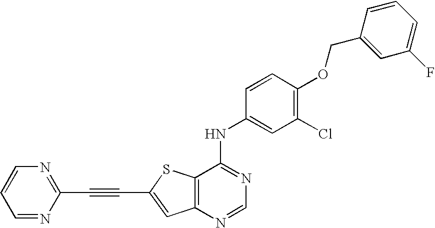

### N-{3-chloro-4-[(3-fluorobenzyl)oxy]phenyl}-6-(pyrimidin-2-ylethynyl)thieno[3,2-d]pyrimidin-4-amine

2-Bromopyrimidine (42 mg, 0.26 mmol), Cul (5 mg, 0.03 mmol), dichlorobis(triphenylphosphine)palladium(II) (8 mg, 0.01 mmol), THF (2 mL), triethylamine (61 μL, 0.44 mmol) and N-{3-chloro-4-[(3-fluorobenzyl)oxy]phenyl}-6-ethynylthieno[3,2-d]pyrimidin-4-amine (90 mg; 0.22 mmol) were combined and heated at 40° C. for 2 h. The mixture was allowed to cool to room temperature and concentrated. The residue was taken up in CHCl₃/MeOH (9:1) and filtered. The filtrate was absorbed onto silica and purified by silica gel chromatography Added the silica gel/crude reaction pad to more silica gel (eluting with 1:1 to 1:4 hexane/ethyl acetate) to yield, after trituration, the title compound (19 mg) as an orange solid, m.p. 236-240° C. ¹H NMR (300 MHz, DMSO-d₆) δ 5.26 (s, 2H), 7.15-7.21 (m, 1H), 7.25-7.34 (m, 3H), 7.43-7.51 (m, 1H), 7.58-7.64 (m, 1H), 7.94 (d, J=2.4 Hz, 1H), 7.98 (s, 1H), 8.63 (s, 1H), 8.91 (d, J=5 Hz), 9.89(s, 1H). HRMS: 488.0755 (MH⁺). Elemental analysis: Found: C, 59.77; H, 3.19; N, 13.48; C₂₅H₁₅ClFN₅OS.3C₃H₈O2.5.H₂0C, 59.85; H, 3.57; N, 13.47.

### Example 78

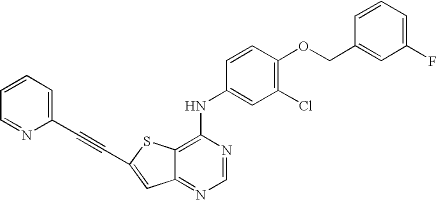

### N-{3-chloro-4-[(3-fluorobenzyl)oxy]phenyl}-6-(pyridin-2-yiethynyl)thieno[3,2-d]pyrimidin-4-amine

A mixture of 2-iodopyridine (60 mg, 0.29 mmol), Cul (5 mg, 0.03 mmol), dichlorobis(triphenylphosphine)palladium(II) (7 mg; 0.01 mmol), THF (2 mL), triethylamine (70 mL; 0.48 mmol) and N-{3-chloro-4-[(3-fluorobenzyl)oxy]phenyl}-6-ethynylthieno[3,2-d]pyrimidin-4-amine (100 mg, 0.24 mmol) was heated and the mixture was worked up according to the foregoing procedure to afford, after silica gel chromatography (1:1 to 1:2hexane/ethyl acetate), 16 mg of the title compound as a yellow solid, mp 242 ° C. HRMS: 487.0808 (MH⁺). Elemental analysis: Found: C, 63.09; H, 3.39; N, 11.18; C₂₆H₁₆ClFN₄OS.0.1C₃H₈O₂.02H₂O C, 63.16; H, 3.51; N, 11.21.

### Example 79

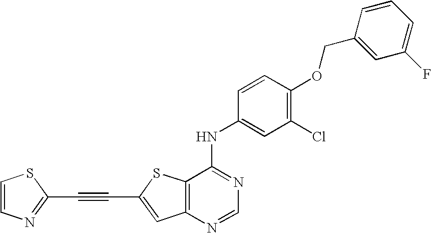

### N-{3-chloro-4-[(3-fluorobenzyl)oxy]phenyl}-6-(1,3-thiazol-2-ylethynyl)thieno[3,2-d]pyrimidin-4-amine

A mixture of 2-bromothiazole (20 μL; 0.23 mmol), Cul (4 mg, 0.02 mmol), dichlorobis(triphenylphosphine)palladium(II) (7 mg, 0.01 mmol), THF (2 mL), triethIylarmine (52 mL; 0.38 mmol), and N-{3-chloro-4-[(3-fluorobenzyl)oxy]phenyl}-6-ethynylthieno[3,2-d]pyrimidin-4-amine (example 77) (77 mg, 0.19 mmol) was heated at 40° C. for 3.5 h. The mixture was worked up according to the above procedure to yield 11 mg of the title compound as a yellow solid, mp 235° C. ¹H NMR (300 MHz, DMSO-d₆) δ 5.26 (s, 2H), 7.18 (m, 1H), 7.25-7.33 (m, 3H), 7.43-7.51(m, 1H), 7.62 (dd, J=9,2.5 Hz, 1H), 7.93 (d, J=2.4 Hz, 1H), 7.96 (s, 1H), 8.08-8.05 (m, 2H), 8.62 (s, 1H), 9.88 (s, 1H).

### Example 80

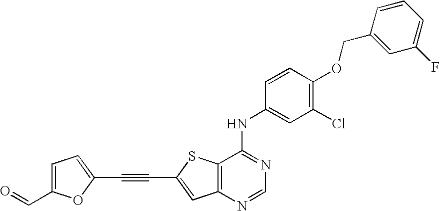

### 5-{[4-({3-chloro-4-[(3-fluorobenzyl)oxy]phenyl}amino)thieno[3,2-d]pyrimidin-6-yl]ethynyl}-2-furaldehyde

A mixture of 5-bromo-2-furaldehyde (41 mg; 0.23 mmol), Cul, (4 mg, 0.02 mmol), dichlorobis(triphenylphosphine)palladium(II) (7 mg; 0.01 mmol), THF (2 mL), triethylamine (55 μL; 0.39 mmol) and N-{3-chloro-4-[(3-fluorobenzyl)oxy]phenyl}-6-ethynylthieno[3,2-d]pyrimidin-4-amine (example 77) (80 mg, 0.20 mmol) was heated at 40° C. for 2 h. The mixture was worked up and purified by silica gel chromatography according to the foregoing procedure to supply the title compound (31 mg) as a yellow solid, mp 229° C.

¹H NMR (300 MHz, DMSO-d₆) δ 5.26 (s, 2H), 7.15-7.21 (m, 1H), 7.25-7.36 (m, 4H), 7.43-7.53 (m, 1H), 7.62 (dd, J=9, 2.5 Hz, 1 H), 7.68 (d, J=3.8 Hz, 1H), 7.93 (d, 1H, J=2.5 Hz), 7.95 (s, 1H), 8.62 (s, 1H), 9.65 (s, 1H), 9.89 (s, 1H)

### Example 81

### Preparation of N-{3-Chloro-4-[(3-fluorobenzyl)oxy]phenyl}-6-ethynylthieno[2,3-d]pyrimidin-4-amine

### Step A

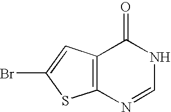

### 6-Bromothieno[2,3-d]pyrimidin-4(3H)-one

To a slurry of commercially available thieno[2,3-d]pyrimidin-4(3H)-one (1.5 g, 9.86 mmol) in glacial acetic acid (26 mL) was added dropwise bromine (1.0 mL, 20 mmol). The dark brown mixture was heated at 80° C. for 1.5 h. The mixture was allowed to cool to ambient temperature and was poured onto a mixture of saturated aqueous NaHCO₃ and ice. The resulting solid was collected by suction filtration, washed with water and dried in vacuo to afford 2.09 of the title compound. ¹H NMR (400 MHz, DMSO-d₆) δ 7.54 (s, 1H), 8.13 (d, 2H, J=3.7 Hz), 12.6 (bs, 1H).

### Step B

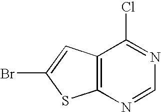

### 6-Bromo-4-chlorothieno[2,3-d]pyrimidine

6-Bromothieno[2,3-d]pyrimidin-4(3H)-one (2.09 g, 9.05 mmol) was covered with phorphorous oxychloride (4.0 mL, 42.9 mmol) and the mixture was heated at 118-120° C. for 2 h. The mixture was allowed to cool to ambient temperature and was poured onto a mixture of saturated aqueous NaHCO₃ and ice. The resulting precipitate was collected by suction filtration and washed with water. The resulting solid was dried in vacuo to afford 2.07 g of the title compound. ¹H NMR (400 MHz, DMSO-d₆) δ 7.88 (s, 1H), 8.93 (s, 1H).

### Step C

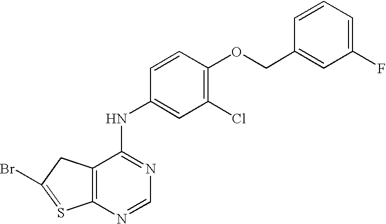

### 6-Bromo-N-{3-chloro-4-[(3-fluorobenzyl)oxy]phenyl}thieno[2,3-d]pyrimidin-4-amine

A mixture of 6-bromo-4-chlorothieno[2,3-d]pyrimidine (2.07 g, 8.29 mmol), 3-chloro-4-[(3-fluorobenzyl)oxy]aniline (2.09 g, 8.29 mmol), triethylamine (2.31 mL, 16.57 mmol) and isopropanol (40 mL) was heated at 85° C. for 16 h. The mixture was allowed to cool to ambient temperature and concentrated to leave a brown residue. The mixture was triturated with ether to afford the title compound (3.34 g) as a tan solid. ¹H NMR (400 MHz, DMSO-d₆) δ 5.24 (s, 2H), 7.18 (m, 1H), 7.26 (d, 1H, J=9.1 Hz), 7.32 (m, 1H), 7.64 (dd, 1H, J=12.1, 2.7 Hz), 8.00 (d, 1H, J=2.5 Hz), 8.02 (s, 1H), 8.48 (s, 1H), 9.63 (s, 1H).

### Step D

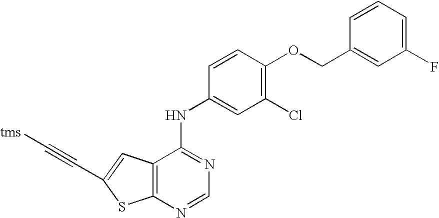

### N-{3-Chloro-4-[(3-fluorobenzyl)oxy]phenyl}-6-[(trimethylsilyl)ethynyl]-4,4a-dihydrothieno[2,3-d]pyrimidin-4-amine

An N₂-flushed flask was charged with 6-bromo-N-{3-chloro-4-[(3-fluorobenzyl)oxy]phenyl}thieno[2,3-d]pyrimidin-4-amine (1.0 g, 2.15 mmol), Cu(I)I (46 mg, 0.24 mmol), dichlorobis(triphenylphosphino)palladium(II) (57 mg, 0.081 mmol), anhydrous THF (13.5 mL), triethylamine (600 μL, 4.3 mmol) and trimethylsilyl acetylene (370 μL, 2.62 mmol) and the resulting mixture was heated at 40° C. for 5 h. The mixture was concentrated with a rotary evaporator and the residue was purified by flash silica gel chromatography (eluting with 5:1 hexanes/ethyl acetate) to afford 623.4 mg of the title compound as a yellow solid. ¹H NMR (400 MHz, DMSO-d₆) δ 0.25 (s, 9H), 5.22 (s, 2H), 7.16 (dt, 1H, J=8.9, 2.5 Hz), 7.24 (d, 1H, J=9.0), 7.29 (m, 1H). 7.43 (m, 1H), 7.62 (dd, 1H, J=8.9, 2.5 Hz), 8.01 (d, 1H, J=2.8 Hz), 8.09 (s, 1H), 8.52 (s, 1H), 9.63 (s, 1H).

### Step E

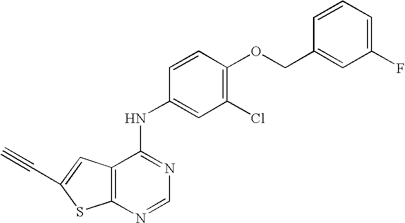

### N-{3-Chloro-4-[(3-fluorobenzyl)oxy]phenyl}-6-ethynylthieno[2,3-d]pyrimidin-4-amine

To a 0° C. solution of N-{3-chloro-4-[(3-fluorobenzyl)oxy]phenyl}-6-[(trimethylsilyl)ethynyl]-4,4a-dihydrothieno[2,3-d]pyrimidin-4-amine (623.4 mg, 1.29 mmol) in anhydrous THF (17 mL) was added 1.0 M TBAF in THF (1.41 mL, 1.41 mmol). The mixture was stirred at 0° C. for 30 min, then partitioned between ethyl acetate and water. The organic layer was separated, dried over Na₂SO₄, filtered and concentrated to give a residue that was purified by silica gel chromatography (eluting with 5:1 hexanes/ethyl acetate) to give 519.3 mg of the title compound as a pale yellow solid, mp 197° C. ¹H NMR (400 MHz, DMSO-d₆) δ 4.89 (s, 1H), 5.27 (s, 2H), 7.21 (t, 1H, J=9.2 Hz), 7.31 (d, 1H, J=9.2 Hz), 7.34-7.36 (m, 1H). 7.45-7.50 (m, 1H), 7.67 (dd, 1H, J=9.0, 2.4 Hz), 8.02 (d, 1H, J=2.4 Hz), 8.10 (s, 1H), 8.56 (s, 1H), 9.75 (s, 1H). ms (MH)+=382.3.

### Example 82

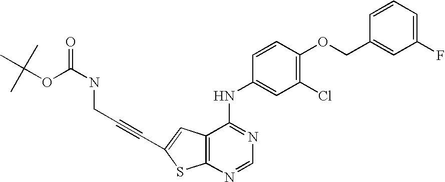

### tert-butyl 3-[4-({3-chloro-4-[(3-fluorobenzyl)oxy]phenyl}amino)thieno[2,3-d]pyrimidin-6-yl]prop-2-ynylcarbamate

6-Bromo-N-{3-chloro-4-[(3-fluorobenzyl)oxy]phenyl}thieno[2,3-d]pyrimidin-4-amine (0.42 g, 0.90 mmol) was combined with N-BOC-propargyl amine (0.17 g, 1.1 mmol), dichlorobis(triphenylphosphine)palladium (II) (0.025 g, 0.036 mmol), Cul (0.017 g, 0.090 mmol), and triethylamine (0.25 mL, 1.8 mmol) in 6 mL THF. The reaction mixture was heated to 60° C. for 1.5 h, then cooled to room temperature and filtered through Celite. Silica gel was added to the filtrate and the solvent was removed in vacuo. The resulting solid was loaded on to a silica gel column and eluted with 1:2 ethyl acetate:hexane to give 0.47 g of product. ¹H NMR (400 MHz, DMSO-d₆) δ 1.39 (m, 9H), 4.04 (m, 2H), 5.22 (s, 2H), 7.16 (m, 1H), 7.26 (m, 3H), 7.43 (m, 2H), 7.62 (m, 1H), 7.97 (br s, 2H), 8.50 (s, 1H), 9.65 (s, 1H). ESI MS m/z 539 (M⁺+H); HPLC RT: 5.41 min.

### Example 83

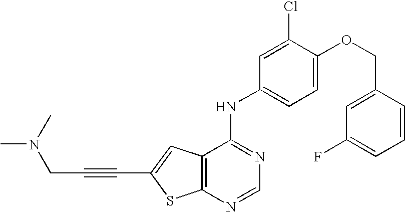

### N-{3-chloro-4-[(3-fluorobenzyl)oxy]phenyl}-6-[3-(dimethylamino)prop-1-ynyl]thieno[2,3-d]pyrimidin-4-amine

The title compound was prepared from 6-bromo-N{3-chloro-4-[(3-fluorobenzyl)oxy]phenyl}thieno[2,3-d]pyrimidin-4-amine by a procedure analogous to example 82 using commercially available N,N-dimethyl-2-propyn-1-amine and known 3-chloro-4-[(3-fluorobenzyl)oxy]aniline. ¹H NMR (400 MHz, DMSO) δ 2.24 (s, 6H), 3.56 (s, 2H), 5.22 (s, 2H), 7.16 (m, 1H), 7.26 (m, 3H), 7.44 (m, 1H), 7.63 (m, 1H), 7.99 (br s, 2H), 8.51 (s, 1H), 9.64 (s, 1H). ESI MS m/z 467 (M⁺+H); HPLC RT: 4.44 min.

### Example 84

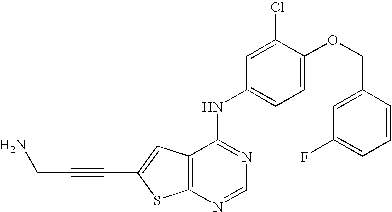

### N-{3-chloro-4-[(3-fluorobenzyl)oxy]phenyl}-6-[3-(methylamino)prop 1-ynyl]thieno[2,3-d]pyrimidin-4-amine

tert-Butyl-3-[4-({3-chloro-4-[(3-fluorobenzyl)oxy]phenyl)amino)thieno[2,3-d]pyrimidin-6-yl]prop-2-ynylcarbamate (0.42 g, 0.78 mmol) was dissolved in trifluoroacetic acid/dichloromethane (3 mL:12 mL) and stirred at room temperature until no starting material was observed by LCMS. Reaction mixture was basified to pH=12 by addition of aqueous 2N sodium hydroxide. The resulting mixture was partitioned, extracted with ethyl acetate, dried over sodium sulfate, filtered, and concentrated to dryness to give a brown solid which was triturated in methanol to yield 0.24 g of title compound as a tan solid. ¹H NMR (400 MHz, DMSO) δ 3.55 (br s, 2H), 5.22 (s, 2H), 7.16 (m, 1H), 7.26 (m, 3H), 7.44 (m, 2H), 7.63 (m, 2H), 7.93 (br s, 1H), 7.99 (br s, 1H), 8.49 (s, 1H), 9.72 (s, 1H). ESI MS mlz 439 (M⁺+H); HPLC RT: 3.07 min.

### Example 85

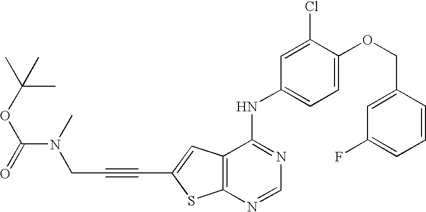

### tert-butyl-3-[4-({3-chloro4-[(3-fluorobenzyl)oxy]phenyl}amino)thieno[2,3-d]pyrimidin-6-yl]prop-2-ynyl(methyl)carbamate

The title compound was prepared from 6-bromo-N-{3-chloro-4-[(3-luorobenzyl)oxy]phenyl}thieno[2,3-d]pyrimidin-4-amine by a procedure analogous to example 82 using known N-methyl-N-(tert-butoxycarbonyl)propargylamine and known 3-chloro-4-[(3-fluorobenzyl)oxy]aniline. ESI MS m/z 553 (M⁺+H); HPLC RT: 4.57 min.

### Example 86

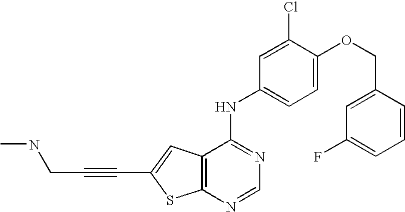

### N-{3chloro-4-[(3-fluorobenzyl)oxy]phenyl}-6-[3-(methylamino)prop-1-ynyl]thieno[2,3-d]pyrimidin-4-amine

The title compound was prepared from tert-butyl-3-[4-({3-chloro-4-[(3-fluorobenzyl)oxy]phenyl}amino)thieno[2,3-d]pyrimidin-6-yl]prop-2-ynyl(methyl)carbamate by a procedure analogous to example 84. ¹H NMR (400 MHz, CDCl₃) δ 2.54 (s, 3H), 3.66 (s, 2H), 5.16 (s, 2H), 6.77 (s, 1H), 6.98 (m, 2H), 7.14 (s, 1H), 7.22 (m, 2H), 7.40 (m, 2H), 7.23 (d, 1H, J=2.4 Hz), 7.99 (br s, 1H), 8.49 (s, 1H). ESI MS m/z 453 (M⁺+H); HPLC RT: 3.09 min.

### Example 87

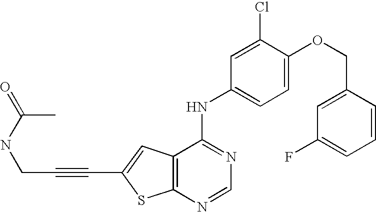

### N-{3-[4-({3-chloro-4-[(3-fluorobenzyl)oxy]pheny}amino)thieno[2,3-d]pyrimidin-6-yl]prop-2-ynyl}acetamide

The title compound was prepared from 6-bromo-N{3-chloro-4-[(3-fluorobenzyl)oxy]phenyl}thieno[2,3-d]pyrimidin-4-amine by a procedure analogous to example 82 using N-2-propynyl-acetamide and known 3-chloro-4-[(3-fluorobenzyl)oxy]aniline. ¹H NMR (400 MHz, DMSO) δ 1.85 (s, 3H), 4.16 (d, 2H, J=1.6 Hz), 5.22 (s, 2H), 7.16 (m, 1H), 7.26 (m, 3H), 7.44 (m, 1H), 7.63 (m, 1H), 7.98 (br s, 2H), 8.44 (br s, 1H), 8.51 (s, 1H), 9.65 (s, 1H). ESI MS m/z 481 (M⁺+H); HPLC RT: 4.08 min.

### Example 88

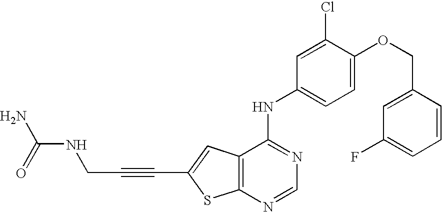

### N-{3-[4-({3-chloro-4-[(3-fluorobenzyl)oxy]phenyl}amino)thieno[2,3-a]pyrimidin-6-yl]prop-2-ynyl}urea

The title compound was prepared from 6-bromo-N-(3-chloro-4-[(3-fluorobenzyl)oxy]phenyl}thieno[2,3-d]pyrimidin-4-amine by a procedure analogous to example 82 using commercially available 2-propynylurea and known 5amino-2-benzyl-1H-benzimidazole. ¹H NMR (400 MHz, DMSO) δ 4.07 (d, 2H, J=5.6 Hz), 5.22 (s, 2H), 5.66 (br s, 1H), 6.42 (br s, 1H), 7.16 (m, 1H), 7.27 (m, 2H), 7.44 (m, 2H), 7.63 (m, 1H), 7.98 (m, 2H), 8.50 (s, 1H), 9.64 (s, 1H). ESI MS m/z 482 (M⁺+H); HPLC RT: 3.98 min.

### Example 89

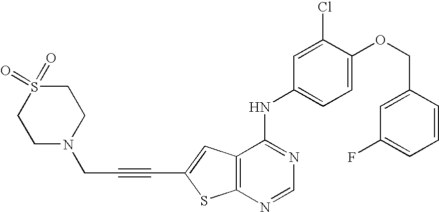

### N-{3-chloro-4-[(3-fluorobenzyl)oxy]phenyl}-6-[3-(1,1-dioxidothiomorpholin-4-yl)prop-1-ynyl]thieno[2,3-d]pyrimidin-4-amine

The title compound was prepared from 6-bromo-N-{3-chloro-4-[(3-fluorobenzyl)oxy]phenyl}thieno[2,3-d]pyrimidin-4-amine by a procedure analogous to example 82 using 4-(2-propynyl)-thiomorpholine 1,1-dioxide and 3-chloro-4-[(3-fluorobenzyl)oxy]aniline. ¹H NMR (400 MHz, DMSO) δ 3.00 (m, 4H), 3.16 (m, 4H), 3.81 (s, 2H), 5.22 (s, 2H), 7.16 (m, 1H), 7.26 (m, 2H), 7.44 (m, 2H), 7.63 (m, 1H), 8.00 (m, 2H), 8.52 (br s, 1H), 9.68 (s, 1H). ESI MS m/z 557 (M⁺+H); HPLC RT: 4.09 min.

### Example 90

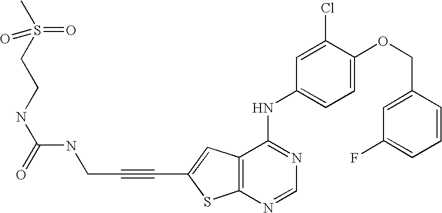

### N-{3-[4-({3-chloro-4-[(3-fluorobenzyl)oxy]phenyl}amino)thieno[2,3-d]pyrimidin-6-yl]prop-2-ynyl}-N′-[2-(methylsulfonyl)ethyl]urea

6-Bromo-N-{3-chloro-4-[(3-fluorobenzyl)oxy]phenyl}thieno[2,3-d]pyrimidin-4-amine (0.040 g, 0.091 mmol) and carbonyidiimidazole (0.015 g, 0.091 mmol) were combined in 1 mL dimethylformamide and the mixture was stirred for 15 min after which time aminoethylmethylsulfone (0.033 g, 0.27 mmol) was added. The mixture was allowed to stir for 18 h after which time the mixture was poured into water, extracted with ethyl acetate, washed with water and brine. The organic layer was dried over sodium sulfate, filtered, and concentrated to a brown oil which was triturated with ether to give 0.031 g of title product as a tan solid. ¹H NMR (400 MHz, DMSO) δ 2.99 (s, 3H), 3.25 (dd, 2H, J=8.8 Hz, 16.8 Hz), 3.45 (dd, 2H, J=8.8 Hz, 16.8 Hz), 4.13 (d, 2H, J=7.6 Hz), 5.24 (s, 2H), 6.30 (t, 1H, J=8.0 Hz), 6.68 (t, 1H, J=7.6 Hz), 7.18 (m, 1H), 7.26 (m, 3H), 7.44 (m, 1H), 7.63 (m, 1H), 7.99 (m, 2H), 8.52 (s, 1H), 9.65 (s, 1H). ESI MS m/z 588 (M⁺+H); HPLC RT: 3.97 min.

### Example 91

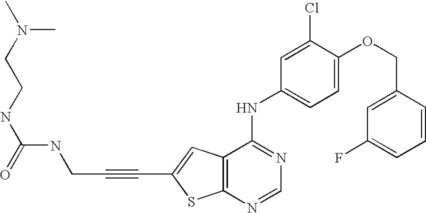

### N-{3-[4-({3-chloro-4-[(3-fluorobenzyl)oxy]phenyl}amino)thieno[2,3-d]pyrimidin-6-yl]prop-2-ynyl)N′-[2-(dimethylamino)ethyl]urea

The title compound was prepared from 6-bromo-N-{3-chloro-4-[(3-fluorobenzyl)oxy]phenyl}thieno[2,3-d]pyrimidin-4-amine by a procedure analogous to example 90 using 2-(N,N-dimethylamino)ethylamine. ¹H NMR (400 MHz, DMSO) δ 2.13 (s, 6H), 2.26 (t, 2H, J=8.4 Hz), 3.10 (m, 2H), 4.11 (d, 2H, J=7.2 Hz), 5.24 (s, 2H), 5.98 (t, 1H, J=7.2 Hz), 6.50 (t, 1H, J=7.2 Hz), 7.18 (m, 1H), 7.26 (m, 3H), 7.44 (m, 1H), 7.63 (m, 1H), 7.99 (m, 2H), 8.52 (s, 1H), 9.65 (s, 1H). ESI MS m/z 553 (M⁺+H); HPLC RT: 3.16 min.

### Example 92

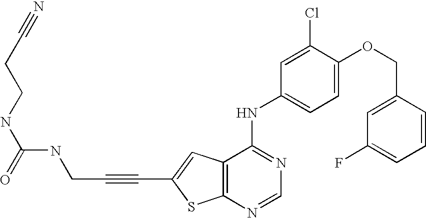

### N-{3-[4-({3-chloro-4-[(3-fluorobenzyl)oxy]phenyl}amino)thieno[2,3-d]pyrimidin-6-yl]prop-2-ynyl}N′-(2-cyanoethyl)urea

The title compound was prepared from 6-bromo-N-{3-chloro-4-[(3-fluorobenzyl)oxy]phenyl}thieno[2,3-d]pyrimidin-4-amine by a procedure analogous to example 90 using 2-cyanoethylamine. ¹H NMR (400 MHz, DMSO) δ 2.62 (t, 2H, J=8.4 Hz), 3.27 (m, 2H), 4.14 (d, 2H, J=7.6 Hz), 5.23 (s, 2H), 6.45 (t, 1H, J=8.0 Hz), 6.62 (t, 1H, J=7.6 Hz), 7.18 (m, 1H), 7.26 (m, 3H), 7.44 (m, 1H), 7.63 (m, 1H), 7.99 (m, 2H), 8.52 (s, 1H), 9.66 (s, 1H). ESI MS m/z 535 (M⁺+H); HPLC RT: 4.03 min.

### Example 93

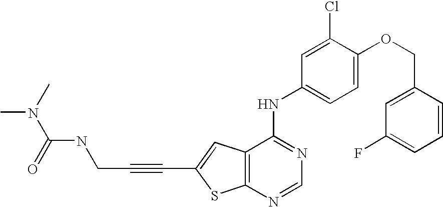

### N′-{3-[4-({3-chloro-4-[(3-fluorobenzyl)oxy]phenyl}amino)thieno[2,3-d]pyrimidin-6-yl]prop-2-ynyl}-N,N-dimethylurea

The title compound was prepared from 6-bromo-N-{3-chloro-4-[(3-fluorobenzyl)oxy]phenyl}thieno[2,3-d]pyrimidin-4-amine by a procedure analogous to example 90 using dimethylamine. ¹H NMR (400 MHz, DMSO) δ 2.80 (s, 6H), 4.11 (d, 2H, J=7.6 Hz), 5.24 (s, 2H), 6.91 (t, 1H, J=7.2 Hz), 7.18 (m, 1H), 7.31 (m, 3H), 7.45 (m, 1H), 7.71 (m, 1H), 8.06 (t, 1H, J=4.0 Hz), 8.14 (s, 1H), 8.52 (s, 1H), 9.92 (s, 1H). ESI MS m/z 510 (M⁺+H); HPLC RT: 4.12 min.

### Example 94

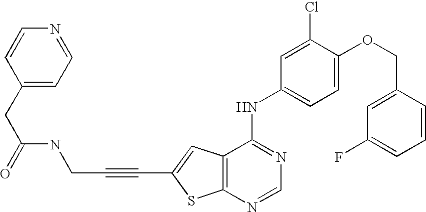

### N-{3-[4-([3-chloro-4-[(3-fluorobenzyl)oxy]phenyl}amino)thieno[2,3-d]pyrimidin-6-yl]prop-2-ynyl}-2-pyridin-4-ylacetamide

6-Bromo-N-{3-chloro-4-[(3-fluorobenzyl)oxy]phenyl}thieno[2,3-d]pyrmidin-4-amine (0.040 g, 0.091 mmol), 4-pyridylacetic acid hydrochloride (0.017 g, 0.10 mmol), triethylamine (0.040 mL, 0.27 mmol), and diethylcyanophosphonate (0.020 mL, 0.15 mmol) were combined in 1 mL dimethylformamide and the mixture was stirred overnight after which time saturated sodium bicarbonate and water were added. The resulting precipitate was filtered, washed with water, and dried in vacuo to give 0.050 g of the title compound as a tan solid. ¹H NMR (400 MHz, DMSO) δ 3.52 (s, 2H), 4.20 (s, 2H), 5.21 (s, 2H), 7.21 (m, 5H), 7.44 (m, 1H), 7.57 (m, 1H), 7.93 (br s, 3H), 8.48 (m, 3H), 8.76 (br s, 1H), 9.70 (br s, 1H). ESI MS m/z 558 (M⁺+H); HPLC RT: 3.62 min.

### Example 95

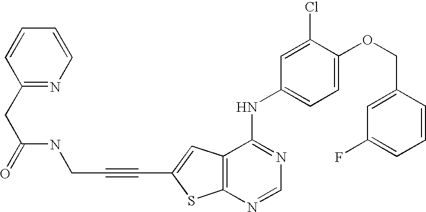

### N-{3-[4-({3-chloro-4-[(3-fluorobenzyl)oxy]phenyl}amino)thieno[2,3-d]pyrimidin-6-yl]prop-2-ynyl}-2-pyridin-2-ylacetamide

Title compound was prepared from 6-bromo-N-{3-chloro-4-[(3-fluorobenzyl)oxy]phenyl}thieno[2,3-d]pyrimidin-4-amine (example 81) by a procedure analogous to example 94 using 2-pyridylacetic acid. ¹H NMR (400 MHz, DMSO) δ 3.65 (s, 2H), 4.20 (s, 2H), 5.21 (s, 2H), 7.25 (m, 6H), 7.44 (m, 1H), 7.58 (m, 1H), 7.72 (m, 1H), 7.94 (m, 2H), 8.45 (br s, 2H), 8.71 (m, 1H), 9.69 (br s, 1H). ESI MS m/z 558 (M⁺+H); HPLC RT: 3.95 min.

### Example 96

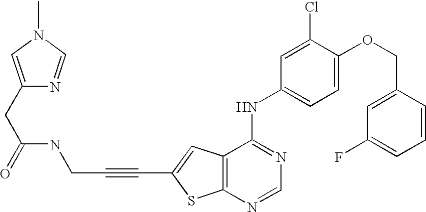

### N-{3-[4-({3-chloro-4-[(3-fluorobenzyl)oxy]pheny{amino)thieno[2,3-d]pyrimidin-6-yl]prop-2-ynyl)-2-(1-methyl-1H-imidazol-4-yl)acetamide

The title compound was prepared from 6-bromo-N-{3-chloro-4-[(3-fluorobenzyl)oxy]phenyl}thieno[2,3-d]pyrimidin-4-amine by a procedure analogous to example 94 using 1-methyl-4-imidazoloacetic acid. ¹H NMR (400 MHz, DMSO) δ 3.32 (s, 3H), 3.57 (s, 2H), 4.18 (br s, 2H), 5.22 (s, 2H), 6.91 (s, 1H), 7.16 (m, 1H), 7.26 (m, 2H), 7.44 (m, 2H), 7.62 (m, 1H), 7.98 (br s, 3H), 8.43 (m, 1H), 8.50 (br s, 1H), 9.65 (m, 1H). ESI MS m/z 561 (M⁺+H); HPLC RT: 3.12 min.

### Example 97

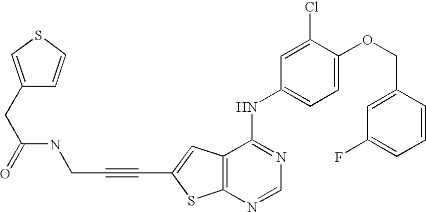

### N-{3-[4-({3-chloro-4-[(3-fluorobenzyl)oxy]phenyl}amino)thieno[2,3-d]pyrimidin-6-yl]prop-2-ynyl)-2-thien-3-ylacetamide

The title compound was prepared from 6-bromo-N-{3-chloro-4-[(3-fluorobenzyl)oxy]phenyl}thieno[2,3-d]pyrimidin-4-amine by a procedure analogous to example 94 using 3-thiopheneacetic acid. ¹H NMR (400 MHz, DMSO) δ 3.47 (s, 2H), 4.18 (br s, 2H), 5.20 (s, 2H), 7.01 (d, 1H, J=4.8 Hz), 7.16 (m, 2H), 7.27 (m, 3H), 7.44 (m, 2H), 7.53 (m, 1H), 7.93 (m, 2H), 8.39 (m, 1H), 8.61 (m, 1H). ESI MS m/z 563 (M⁺+H); HPLC RT: 4.21 min.

### Example 98

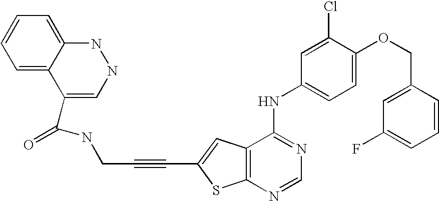

### N-{3-[4-({-3-chloro-4-[(3-fluorobenzyl)oxy]phenyl}amino)thieno[2,3-d]pyrimidin-6-yl]prop-2-ynyl}cinnoline-4-carboxamide

The title compound was prepared from 6-bromo-N{3-chloro-4-[(3-fluorobenzyl)oxy]phenyl}thieno[2,3-d]pyrimidin-4-amine by a procedure analogous to example 94 using cinnoline 4-carboxylic acid. ¹H NMR (400 MHz, DMSO) δ 4.54 (s, 2H), 5.23 (s, 3H), 7.16 (m, 1H), 7.26 (m, 3H), 7.44 (m, 1H), 7.62 (m, 1H), 7.98 (m, 4H), 8.28 (m, 1H), 8.55 (m, 2H), 9.45 (s, 1H), 9.66 (br s, 1H). ESI MS m/z 593 (M-H); HPLC RT: 4.15 min.

### Example 99

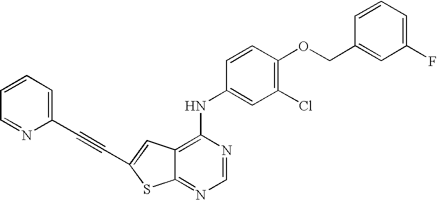

### N-{3-chloro-4-[(3-fluorobenzyl)oxy]phenyl}-6-(pyridin-2-ylethynyl)thieno[2,3-d]pyrimidin-4-amine

N-{3-chloro-4-[(3-fluorobenzyl)oxy]phenyl}-6-ethynylthieno[2,3-d]pyrimidin-4-amine (100 mg, 0.244 mmol), 2-iodopyridine (55 μL, 108 mg, 0.528 mmol), triethylamine (61 μL, 0.44 mmol), Cul (11 mg, 0.058 mmol), and dichlorobis(triphenylphosphine)palladium (II) (8.2 mg, 0.012 mmol) were placed in a N₂-flushed reaction vessel and THF (1.5 mL) was added. The resulting mixture was heated at 40° C. for 1-5 h until no starting material was observed by TLC. The mixture was concentrated, dissolved in 5:1 CHCl₃/MeOH and filtered. The filtrate was absorbed onto silica gel and purified by silica gel chromatography eluting with hexanes/ethyl acetate to afford, after concentration of the relevant fractions 75.3 mg of the title compound as a light yellow powder, mp 180° C. HRMS: 486.0717 (MH)⁺. ¹H NMR (400 MHz, DMSO-d₆) δ 5.23 (s, 2H), 7.16 (t, 1H, J=9.2 Hz), 7.26 (d, 1H, J=7.7 Hz), 7.30-7.32 (m, 2H), 7.42-7.47 (m, 1H), 7.65 (dd, 1H, J=8.9, 2.5 Hz), 7.71 (d, 1H, J=7.7 Hz), 7.88 (t, 1H, J=7.7 Hz), 8.01 (d, 1H, J=2.4 Hz), 8.21 (s, 1H), 8.54 (s, 1H), 8.64 (d, 1H, J=4.3 Hz), 9.78 (s, 1H).

### Example 100

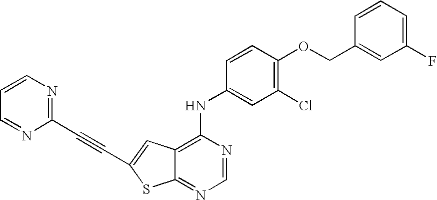

### N-{3-chloro-4-[(3-fluorobenzyl)oxy]phenyl)-6-(pyrimidin-2-ylethynyl)thieno[2,3-d]pyrimidin-4-amine

The procedure of example 99 was followed using N-{3-chloro-4-[(3-fluorobenzyl)oxy]phenyl)6-ethynylthieno[2,3-d]pyrimidin-4-amine (88 mg, 0.215 mmol), 2-bromopyrimidine (42 mg, 0.264 mmol), triethylamine (55 μL, 0.40 mmol), Cu(l)l (4.5 mg, 0.024 mmol), dichlorobis(triphenylphosphine)palladium (II) (6.6 mg, 0.009 mmol) in THF (1.5 mL). Workup and silica gel chromatography afforded 59.7 mg of the title compound as a light yellow powder, mp 225° C. HRMS: 488.0742 (MH)⁺. ¹H NMR (400 MHz, DMSO-d₆) δ 5.23 (s, 2H), 7.17 (t, 1H, J=8.4 Hz), 7.26 (d, 1H, J=9.2 Hz), 7.30-7.32 (m, 2H), 7.42-7.48 (m, 1H), 7.55 (t, 1H, J=7.64 (dd, 1H, J=8.8, 2.5 Hz), 8.00 (d, 1H, J=2.6Hz), 8.30 (s, 1H), 8.56 (s, 1H), 8.87 (s, 1H), 8.88 (s, 1H), 9.82 (s, 1H).

### Example 101

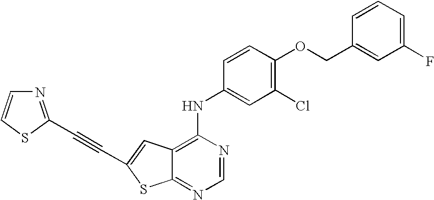

### N-{3-chloro-4-[(3-fluorobenzyl)oxy]phenyl}-6-(1,3-thiazol-2-ylethynyl)thieno[2,3-d]pyrimidin-4-amine

The general procedure of example 99 was followed using N-{3-chloro-4-[(3-fluorobenzyl)oxy]phenyl}-6-ethynylthieno[2,3-d]pyrimidin-4-amine (101.4 mg, 0.247 mmol), 2-bromothiazole (48 μL, 87.4 mg, 0.528 mmol), triethylamine (61 μL, 0.44 mmol), Cu(I)I (8.6 mg, 0.045 mmol), dichlorobis(triphenylphosphine)palladium (II) (8.2 mg, 0.012 mmol) in THF (1.5 mL). Workup and silica gel chromatography supplied 13.1 mg of the title compound as a dark yellow powder, mp 204° C. HRMS: 493.0341 (MH)⁺. ¹H NMR (400 MHz, DMSO-d₆) δ 5.23 (s, 2H), 7.16 (t, 1H, J=8.8 Hz), 7.26 (d, 1H, J=9.7 Hz), 7.30-7.31 (m, 2H), 7.42-7.48 (m, 1H), 7.64 (dd, 1H, J=9.0, 2.4 Hz), 8.00-8.03 (m, 3H), 8.27 (s, 1H), 8.56 (s, 1H), 9.81 (s, 1H).

### Example 102

### Preparation of N-(1-benzyl-1H-indazol-5-yl)-6-ethynylthieno[2,3-d]pyrimidin-4-amine

### Step A

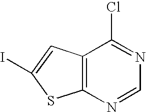

### 6-Iodo4-chlorothieno[2,3-d]pyrimidine

A solution of diisopropylamine (0.841 mL, 6.0 mmol) in THF (10 mL) under N₂ was treated with n-BuLi (2.4 mL of a 2.5 M solution, 42.9 mmol) and the mixture was stirred at 0° C. for 10 min. The solution was cooled to −78° C. and a solution of 4-chlorothieno[2,3-d]pyrimidine (1.02 g, 6.0 mmol) in THF (10 mL) was added dropwise. The mixture was stirred at −78° C. for 40 min, then treated with a solution of iodine (1.52 g, 6.0 mmol) in THF (10 mL). The resulting mixture was allowed to warm to ambient temperature and quenched with water and extracted with CH₂Cl₂. The organic extract was washed with brine, dried over Na₂SO₄, filtered and concentrated to give the crude material. Purification by silica gel chromatography (7.5:1 hexane/ethyl acetate) supplied 1.12 g of the title compound as a yellow solid. ¹H NMR (400 MHz, CDCl₃) δ 7.51 (s, 1H), 8.85 (s, 1H).

### Step B

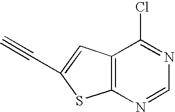

### 4-Chloro-6-ethynylthieno[2,3-d]pyrimidine

A reaction vessel flushed with N₂ was charged with 6-iodo-4-chlorothieno[2,3-d]pyrimidine (950 mg, 3.2 mmol), Cul (61 mg, 0.32 mmol), dichlorobis(triphenylphosphine)palladium(II) (84 mg, 0.12 mmol), triethylamine (0.90 mL, 6.4 mmol), trimethylsilylacetylene (0.38 g, 0.54 mL, 3.84 mmol) and anhydrous THF (20 mL). The mixture was heated at 55° C. for 45 min. The mixture was concentrated by rotary evaporator and purified by silica gel chromatography (30:1 hexane/ethyl acetate eluant) to supply, after concentration of the relevant fractions, the silylacetylene intermediate. This material was taken up in THF (18 mL) and cooled to 0° C. A solution of 1.0 M TBAF in THF (1.66 mL, 1.66 mmol) was added and the mixture was stirred at 0° C. for 10 min. The mixture was partitioned between water and ethyl acetate. The organic layer was separated, dried over Na₂SO₄, filtered and concentrated. The residue was purified by silica gel chromatography (20:1 hexane/ethyl acetate) to afford 203.8 mg of the title compound as 1:1.5 mixture of 4-chloro-6-ethynylthieno[2,3-d]pyrimidine: 4-fluoro-6-ethynylthieno[2,3-d]pyrimidine. ¹H NMR (400 MHz, CDCl₃)δ 3.59 (s, 0.4H), 3.61 (s, 0.6H), 7.57 (s, 0.4H) 7.59 (s, 0.6H), 8.78 (s, 0.4H), 8.87 (0.6H).

### Step C

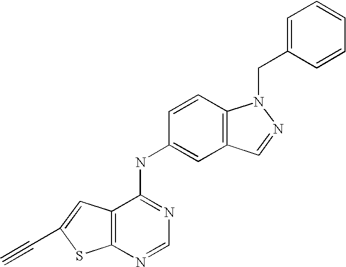

### N-(1-benzyl-1H-indazol-5-yl)-6-ethynylthieno[2,3-d]pyrimidin-4-amine

A mixture of 1-benzyl-1H-indazol-5-amine (80.3 mg, 0.36 mmol) and 4-chloro-6-ethynylthieno[2,3-d]pyrimidine (67.9 mg, 0.36 mmol, used as a 1.5:1 mixture of 4-fluoro-6-ethynylthieno[2,3-d]pyrimidine: 4-chloro-6-ethynylthieno[2,3-d]pyrimidine) was heated at 50-60° C. in isopropanol (2.0 mL) for 19 h. The mixture was allowed to cool to ambient temperature, then partitioned between saturated aqueous NaHCO₃ and ethyl acetate. The organic layer was separated, dried over Na₂SO₄, filtered and concentrated to supply a residue that was purified by silica gel chromatography (2:1 hexane/ethyl acetate) to give 112.9 mg of the title compound as a light tan solid, mp 195° C. ¹H NMR (400 MHz, DMSO-d₆) δ 4.88 (s, 1H), 5.69 (s, 2H), 7.27 (t, 1H, 7.5 Hz), 7.28-7.34 (m, 2H), 7.64 (d, 1H, J=12.0Hz), 7.75 (d, 1H, J=12.0)8.14(s, 1H), 8.16 (s, 1H), 8.24 (s, 1H), 8.52 (s, 1H), 9.83 (s, 1H).

### Example 102

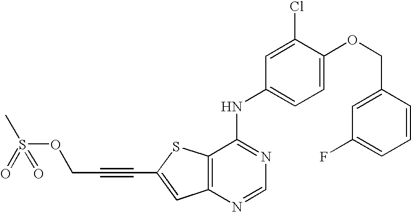

### 3-[4-({3-chloro-4-[(3-fluorobenzyl)oxy]phenyl}amino)thieno[3,2-d]pyrimidin-6-yl]prop-2-ynyl methanesulfonate

3-[4-({3-Chloro4-[3-fluorobenzyl)oxy]phenyl}amino)thieno[3,2-d]pydmidin-6-yl]prop-2-yn-1-ol (0.40 g, 0.91 mmol) was dissolved in 8 mL DMA and 0.5 mL diisopropylethylamine. Methanesulfonic anhydride (0.25 g, 1.5 mmol) was added and the reaction mixture was heated to 60 C for 0.25 h. The reaction mixture was poured into 50 mL water and extracted with tert-butylmethyl ether (3×50 mL). The ethereal layer was washed with water (2×50 mL) and brine (50 mL), dried over sodium sulfate, filtered and concentrated to give 0.42 g mesylate product. HPLC RT: 3.81 min. HRMS: 518.0405 (MH⁺) ¹H NMR (CDCl₃) δ 8.76 (s,1H), 7.61 (d,1H), 7.55 (s,1H), 7.41-7.33 (m 2H), 7.26-7.21 (m, 2H), 7.07-6.99 (m, 2H), 6.84 (br s, 1H), 5.22 (s, 2H), 5.11 (s, 2H), 3.15 (s, 3H).

### Example 103

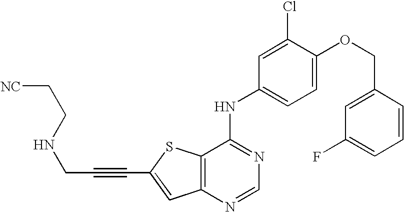

### 3-({3-[4-({3chloro-4-[(3-fluorobenzyl)oxy]phenyl}amino)thieno[3,2-d]pyrimidin-6-yl]prop-2-ynyl}amino)propanenitrile

3-[4-({3-chloro-4-[(3-fluorobenzyl)oxy]phenyl}amino)thieno[3,2-d]pyrimidin-6-yl]prop-2-ynyl methanesulfonate (0.040 g, 0.077 mmol) and cyanoethylamine (0.017 mL, 0.231 mmol) were dissolved in 1 mL DMA and 0.1 mL diisopropylethylamine. The reaction mixture was heated to 60 C for 0.5 h. The reaction mixture was poured into 30 mL water and extracted with tert-butylmethyl ether (2×30 mL). The ethereal layer was washed with water (2×30 mL) and brine (30 mL), dried over sodium sulfate, filtered and concentrated. Column chromatography (5% MeOH in CH₂Cl₂) gave 0.022 g product. HPLC RT: 3.15 min. HRMS: 492.1081 (MH⁺) ¹H NMR (CDCl₃) δ 8.65 (s,1H), 7.61 (d,1H), 7.43 (s,1H), 7.39-7.34 (m, 2H), 7.26-7.21 (m, 2H), 7.04-6.97 (m, 2H), 5.20 (s, 2H), 3.77 (s, 2H), 3.07 (t, 2H), 2.59 (t, 2H), —NH signals not observed due to line broadening.

### Example 104

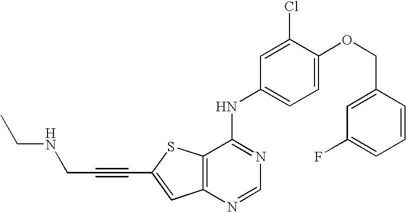

### N-{3-chloro-4-[(3-fluorobenzyl)oxy]phenyl}-6-[3-(ethylamino)prop-1-ynyl]thieno[3,2-d]pyrimidin4-amine

Compound was prepared from and ethylamine 3-[4-({3-chloro-4-[(3-fluorobenzyl)oxy]phenyl}amino)thieno[3,2-d]pyrimidin-6-yl]prop-2-ynyl methanesulfonate following a procedure analogous to Example 103. HPLC RT: 2.85 min. HRMS: 467.1105 (MH⁺).

### Example 105

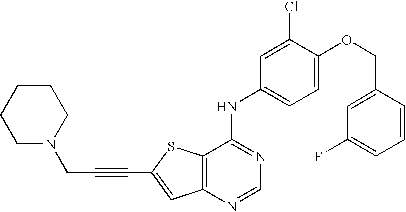

### N-{3-chloro-4-[(3-fluorobenzyl)oxy]phenyl}-6-(3-piperidin-1-ylprop-1-ynyl)thieno[3,2-d]pyrimidin-4-amine

Compound was prepared from 3-[4-({3-chloro-4-[(3-fluorobenzyl)oxy]phenyl}amino)thieno[3,2-d]pyrimidin-6-yl]prop-2-ynyl methanesulfonate and cyclohexylamine following a procedure analogous to Example 103. HPLC RT: 2.95 min. HRMS: 507.1429 (MH⁺).

### Example 106

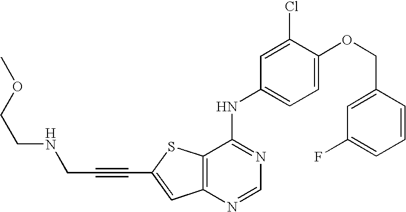

### N-{3-chloro-4-[(3-fluorobenzyl)oxy]phenyl}-6-{3-[(2-methoxyethyl)amino]prop-1-ynyl)thieno[3,2-d]pyrimidin-4-amine

Compound was prepared from 3-[4-({3-chloro-4-[(3-fluorobenzyl)oxy]phenyl}amino)thieno[3,2-d]pyrimidin-6-yl]prop-2-ynyl methanesulfonate and 2-methoxyethylamine following a procedure analogous to Example 103. HPLC RT: 2.89 min. HRMS: 497.1217 (MH⁺).

### Example 107

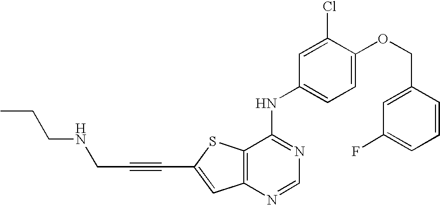

### N-{3-chloro-4-[(3-fluorobenzyl)oxy]phenyl}-6-[3-(propylamino)prop-1-ynyl]thieno[3,2-d]pyrimidin-4-amine

Compound was prepared from 3-[4-({3-chloro-4-[(3-fluorobenzyl)oxy]phenyl}amino)thieno[3,2-d]pyrimidin-6-yl]prop-2-ynyl methanesulfonate and propylamine following a procedure analogous to Example 103. HPLC RT: 2.91 min. HRMS: 481.1260 (MH⁺).

### Example 108

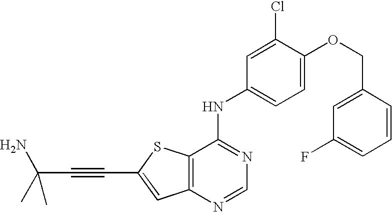

### 6-(3-amino-3-methylbut-1-ynyl)-N-{3-chloro-4-[(3-fluorobenzyl)oxy]phenyl}thieno[3,2-d]pyrimidin4-amine

6-Bromo-N-{3-chloro-4-[(3-fluorobenzyl)oxy]phenyl}thieno[3,2-d]pyrimidin-4-amine hydrochloride (0.060 g, 0.120 mmol) was combined with 1,1-dimethylpropargyl amine (0.027 mL, 0.240 mmol), dichlorobis(triphenylphosphine) palladium (II) (0.005 g), copper (I) iodide (0.002 g, 0.012 mmol), and triethylamine (0.042 mL, 0.300 mmol) in 1 mL THF. The reaction mixture was heated to 60 C for 0.5 h, then cooled to room temperature. Tan precipitate was filtered and rinsed with THF (2 mL) and water (3 mL). Dried in vacuo to give 0.030 g product. HPLC RT: 2.93 min. HRMS: 467.1126 (MH⁺) ¹H NMR (DMSO-d₆) δ 9.89 (s,1H), 8.73 (br s, 2H), 8.59 (s,1 H), 7.91 (d, 1H), 7.69 (s, 1H), 7.62 (dd,1H), 7.50-7.43 (m,1H), 7.32-7.15 (m, 4H), 5.25 (s, 2H), 3.33 (s, 6H).

### Example 109

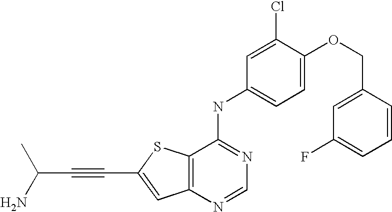

### (R,S)-6-(3-aminobut-1-ynyl)-N-{3-chloro-4-[(3-fluorobenzyl)oxy]phenyl}thieno[3,2-d]pyrimidin-4-amine

### Step A

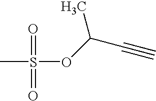

### (R,S)-1-Methylbut-2-ynyl Methanesulfonate

(R,S)3-Butyn-2-ol (2.0 mL, 25.5 mmol) was treated with methanesulfonyl chloride (3.0 mL, 38.2 mmol) and triethylamine (7.2 mL, 51.0 mmol) in 200 mL dichloromethane at −78° C. for 1 h. The reaction was quenched with 50 mL saturated sodium bicarbonate then diluted with 100 mL water. Dichloromethane was removed with a rotary evaporator and the aqueous phase was extracted with ether (2×150 mL). Ether layer was washed with brine and dried over sodium sulfate. Ether was removed with a rotary evaporator to give 3.5 g of the title compound as a colorless oil. ¹H NMR (CDCl3) δ 5.29 (dq, 1H), 3.68 (s, 3H), 2.71 (d, 1H), 1.67 (d, 3H).

### Step B

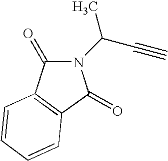

### (R,S)-2-(1-Methylprop-2-ynyl)-1H-isoindole-1,3(2H)dione

1-Methylbut-2-ynyl methanesulfonate (3.0 mL, 23.3 mmol) and potassium phthalimide (5.1 g, 27.5 mmol) were taken up in 200 mL DMF and heated to 75° C. for 16 h. The reaction mixture was poured into 300 mL water and extracted with ethyl acetate (3×150 mL). The combined organic extracts were washed with water (2×100 mL) and 100 mL brine, then dried over sodium sulfate. The solvent was removed with a rotary evaporator and the crude product was adsorbed onto silica gel. Column chromatography with 25-50% ethyl acetate in hexane gradient gave 2.1 g of the title compound as a white solid. LC-MS: 200 (MH+) HPLC RT: 2.85 min.

### Step C

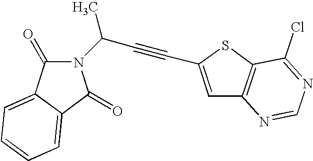

### 2-[3-(4-Chlorothieno[3,2-d]pyrimidin-6-yl)-1-methylprop-2-ynyl]-1H-isoindole-1,3(2H)-dione

6-Bromo-4-chlorothieno[3,2-d]pyrimidine (2.5 g, 10.0 mmol) was combined with 2-(1-methylprop-2-ynyl)-1H-isoindole-1,3(2H)-dione (2.0 g, 10.0 mmol), dichlorobis(triphenylphosphine) palladium (II) (0.38 g), copper (I) iodide (0.38 g, 2.0 mmol), and triethylamine (2.8 mL, 20.2 mmol) in 80 mL THF. The reaction mixture was heated to 60° C. for 0.5 h, then cooled to room temperature and filtered through Celite. Silica gel was added to the filtrate and the solvent was removed with a rotary evaporator. The resulting solid was loaded on to a column of silica gel and eluted with 10-50% ethyl acetate in hexane gradient to give 2.9 g of the title compound as a tan solid. LC-MS: 368 (MH+) HPLC RT: 4.22 min.

### Step D

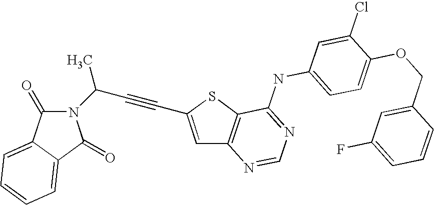

### 2-{3-[4-[(3-Chloro-4-[(3-fluorobenzyl)oxy]phenyl}amino)thieno[3,2-d]pyrimidin-6-yl]-1-methylprop-2-ynyl}-1H-isoindole-1,3(2H)-dione

2-[3-(4-Chlorothieno[3,2-d]pyrimidin-6-yl)1-methylprop-2-ynyl]-1H-isoindole-1,3(2H)-dione (0.30 g, 0.82 mmol) was combined with 3-chloro-4-[(3-fluorobenzyl)oxy]aniline (0.21 g, 0.82 mmol) in 8 mL isopropyl alcohol. The mixture was heated to 65° C. for 4 h. A tan solid precipitated and the reaction mixture was cooled to room temperature. The solid was filtered, rinsed with 10 mL ethyl acetate, and dried with a rotary evaporator to give 0.48 g yellow solid. This crude material was covered with 2 mL IPA and 10 mL saturated sodium bicarbonate and stirred vigorously for 3 h. The heterogeneous mixture was then filtered and dried to give 0.41 g of the title compound as a tan solid. LC-MS: 584 (MH+) HPLC RT: 4.53 min.

### Step E

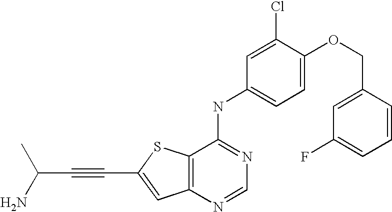

### (R,S)-6-(3-Aminobut-1-ynyl)-N-{3-chloro-4-[(3-fluorobenzyl)oxy]phenyl}thieno[3,2-d]pyrimidin4-amine

2-{3-[4-({3-Chloro-4-[(3-fluorobenzyl)oxy]phenyl]amino)thieno[3,2-d]pyrimidin-6-yl]-1-methylprop-2-ynyl}-1H-isoindole-1,3(2H)-dione was taken up in 40 mL methylamine (2.0 M in methanol) solution and heated to 65° C. for 1.5 h. Methanol was removed with a rotary evaporator and crude was taken up in 50 mL water. The mixture was extracted with ethyl acetate (2×50 mL). The combined organic layers were washed with brine and dried over sodium sulfate. The crude product was adsorbed to silica gel with a rotary evaporator. Flash chromatography (5-15% gradient methanol in ethyl acetate+1% triethylamine) gave 0.26 g of the title compound as a yellow solid. LC-MS: 453 (MH+) HPLC RT: 2.99 min. ¹H NMR (DMSO-d₆): δ 9.69 (br s, 1H), 8.55 (s, 1H), 7.90 (d, 1H), 7.59 (dd,1H), 7.51 (s,1 H), 7.49-7.43 (m,1H), 7.32-7.29 (m, 2H), 7.24 (d, 1H), 7.20-7.15 (m, 1H), 5.24 (s, 2H), 3.89 (q,1H), 2.08 (br s, 2H) 1.32 (d, 3H).

### Example 110

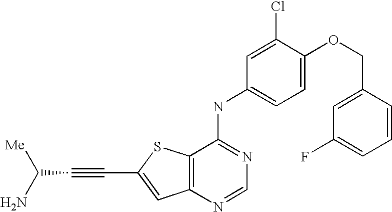

### (R)-6-(3-Aminobut-1-ynyl)-N-}3-chloro-4-[(3-fluorobenzyl)oxy]phenyl}thieno[3,2]pyrimidin-4-amine

The title compound was prepared from 6-bromo-4-chlorothieno[3,2-d]pyrimidine and commercial (S)-(−)-3-butyne-2-ol by a procedure analogous to that used to prepare foregoing Example 109. LC-MS: 453 (MH+) HPLC RT: 2.99 min. ¹H NMR (DMSO-d₆): δ 9.69 (br s, 1H), 8.55 (s, 1H), 7.90 (d, 1H), 7.59 (dd, 1H), 7.51 (s, 1H), 7.49-7.43 (m, 1H), 7.32-7.29 (m, 2H), 7.24 (d, 1H), 7.20-7.15 (m, 1H), 5.24 (s, 2H), 3.89 (q, 1H), 2.12 (br s, 2H) 1.32 (d, 3H). Confirmation of enantiomeric purity (>10:1) was carried out by chiral HPLC analysis of the precursor (R)-2-{3-[4-({3-chloro-4-[(3-fluorobenzyl)oxy]phenyl}amino)thieno[3,2-d]pyrimidin-6-yl]-1-methylprop-2-ynyl}-1H-isoindole-1,3(2H)-dione. ChiralCel RT: 3.53 min.

### Example 111

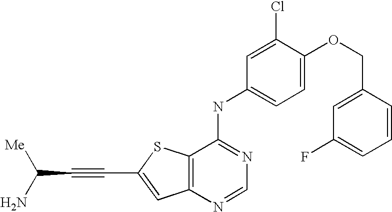

### (S)-6-(3-Aminobut-1-ynyl)-N-(3-chloro-4-[(3-fluorobenzyl)oxy]phenyl}thieno[3,2-d]pyrimidin-4-amine

The title compound was prepared from 6-bromo-4-chlorothieno[3,2-d]pyrimidine and commercial (R)-(−)-3-butyne-2-ol by a procedure similar to that used to prepare Example 109. LC-MS: 453 (MH+) HPLC RT: 2.99 min. ¹H NMR (DMSO-d₆): δ 9.69 (br s,1H), 8.55 (s, 1H), 7.90 (d, 1H), 7.59 (dd, 1H), 7.51 (s, 1H), 7.49-7.43 (m, 1H), 7.32-7.29 (m, 2H), 7.24 (d, 1H), 7.20-7.15 (m, 1H), 5.24 (s, 2H), 3.89 (q, 1H), 2.12 (br s, 2H) 1.32 (d, 3H). Confirmation of enantiomeric purity (>10:1) was carried out by chiral HPLC analysis of the precursor (S)-2-{3-[4-({3-chloro-4-[(3-fluorobenzyl)oxy]phenyl}amino)thieno[3,2-d]pyrimidin-6-yl]-1-methylprop-2-ynyl)1H-isoindole-1,3(2H)-dione. ChiralCel RT: 3.79 min.

### Example 112

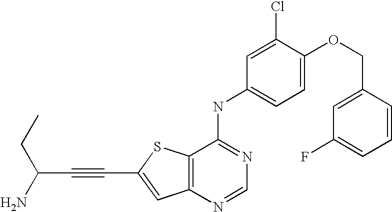

### (R,S)-6-(3-Aminopent-1-ynyl)-N-{3-chloro-4-[(3-fluorobenzyl)oxy]phenyl}thieno[3,2-d]pyrimidin-4-amine

The title compound was prepared from 6-bromo-4-chlorothieno[3,2-d]pyrimidine and 3-pentynol by a procedure similar to Example 109. LC-MS: 467 (MH+) HPLC RT: 3.10 min. ¹H NMR (CDCl₃): δ 8.64 (s, 1H), 7.61 (d, 1H), 7.40-7.33 (m, 3H), 7.26-7.22 (m, 2H), 7.06-7.01 (m, 2H), 6.98 (d,1H), 5.20 (s, 2H), 3.77 (t, 1H), 1.78-1.68 (m, 2H), 1.07 (t, 3H).

The following additional examples 113-125 were prepared in a manner analogous to that used to produce Example 109 and were characterized to be the indicated compound:

### Example 113

### (R,S)-6-(3-aminobut-1-ynyl)-N-[3-chloro-4-(1-naphthyloxy)phenyl]thieno[3,2-d]pyrimidin-4-amine

### Example 114

### (R,S)-6-(3-aminobut-1-ynyl)-N-(2-benzyl-1H-benzimidazol-5-yl)thieno[3,2-d]pyrimidin-4-amine

### Example 115

### (R,S)-6-(3-aminobut-1-ynyl)-N-[1-(pyridin-3-ylmethyl)-1H-indol-5-yl]thieno[3,2-d]pyrimidin-4-amine

### Example 116

### (R,S)—N⁴-[6-(3-aminobut-1-ynyl)thieno[3,2-d]pyrimidin4-yl]-2-chloro-N¹-(3-fluorobenzyl)benzene-1,4-diamine

### Example 117

### (R,S)-6-(3-aminobut-1-ynyl)-N-[1-(3-fluorobenzyl)1H-indazol-5-yl]thieno[3,2-d]pyrimidin-4-amine

### Example 118

### (R,S)-6-(3-aminobut-1-ynyl)-N-{3-fluoro-4-[(3-fluorobenzyl)oxy]phenyl}thieno[3,2-d]pyrimidin4-amine

### Example 119

### (R,S)-6-(3-Aminobut-1-ynyl)-N-(4-benzylphenyl)thieno[3,2-d]pyrimidin-4-amine

### Example 120

### (R,S)-6-(3-Aminobut-1-ynyl)-N-[1-(2-fluorobenzyl)-1H-indazol-5-yl]thieno[3,2-d]pyrimidin-4-amine

### Example 121

### (R,S)-6-(3-Aminobut-1-ynyl)-N-[2-(2-fluorobenzyl)-1H-benzimidazol-5-yl)thieno[3,2d]pyrimidin-4-amine

### Example 122

### (R,S)-6-(3-aminobut-1-ynyl)-N-[1-(2,5-difluorobenzyl)-1H-indol-5-yl]thieno[3,2-d]pyrimidin-4-amine

### Example 123

### (R,S)-6-(3-aminobut-1-ynyl)-N-(1-benzyl-1H-indol-5-yl)thieno[3,2-d]pyrimidin-4-amine

### Example 124

### (R,S)-6-(3-aminobut-1-ynyl)-N-(1-benzyl-1H-indazol-5-yl)thieno[3,2-d]pyrimidin4-amine

### Example 125

### (R,S)-6-(3-aminobut-1-ynyl)-N-[2-(3-fluorobenzyl)-1H-benzimidazol-5-yl]thieno[3,2-d]pyrimidin4-amine

### Example 126

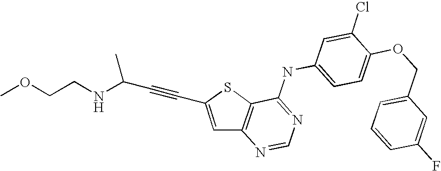

### (R,S)—N-{3-chloro-4-[(3-fluorobenzyl)oxy]phenyl}-6-{3-[(2-methoxyethyl)amino]but-1-ynyl}thieno[3,2-d]pyrimidin4-amine

### Step A

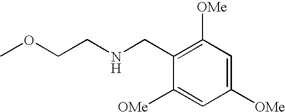

### 2-Methoxy-N-(2,4,6-trimethoxybenzyl)ethanamine

2-Methoxyethylamine (2.0 mL, 23.0 mmol) and 2,4,6-trimethoxybenzaldehyde (4.2 g, 21.2 mmol) were refluxed in 30 mL benzene for 1.5 h where water was removed by Dean-Stark apparatus. The reaction was cooled and benzene was removed with a rotary evaporator to give a white solid. This material was dissolved in 25 mL methanol, cooled in an ice bath, and sodium borohydride (1.3 g, 35 mmol) was added over 3 min. The ice bath was removed and the reaction stirred at ambient temperature for 16 h. The solvent was removed with a rotary evaporator and the residue was taken up in 70 mL water and extracted with (3×50 mL) ethyl acetate. The combined organic layers were washed with brine and dried over sodium sulfate. The solvent was evaporated to give 5.5 g of the title compound as a faintly brown, clear oil. ¹H NMR (CDCl₃) δ 6.12 (d, 2H), 4.72-4.69 (m, 1H), 3.83-3.78 (m, 11H), 3.49 (t, 2H), 3.32 (s, 3H), 2.74 (t, 2H).

### Step B

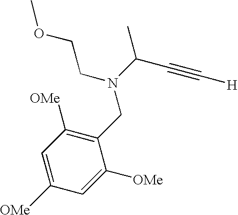

### (2R,S)—N-(2-methoxyethyl)-N-(2,4,6-trimethoxybenzyl)but-3-yn-2-amine

2-Methoxy-N-(2,4,6-trimethoxybenzyl)ethanamine_(0.99 g, 3.9 mmol) was dissolved in 20 mL DMF and potassium carbonate (1.1 g, 7.8 mmol) was added. (2R,S)-1-methylbut-2-ynyl methanesulfonate (0.5 mL, 3.9 mmol) was added and the reaction was heated to 90° C. for 2.5 h. The reaction mixture was poured into 50 mL water and extracted with (3×40 mL) ethyl acetate. The combined organic layers were washed with 50 mL each water and brine, and dried over sodium sulfate. The crude product was passed through a plug of basic alumina with 50% ethyl acetate in hexane to give 1.1 g of the title compound as a clear brown oil. ¹H NMR (CDCl₃) δ 6.12 (d, 2H), 3.83-3.78 (m, 11H), 3.73-3.65 (m, 1H), 3.49 (t, 2H), 3.33 (s, 3H), 2.83-2.71 (m, 1H), 2.69-2.59 (m,1H), 2.21, (d, 1H), 1.32 (d, 3H).

### Step C

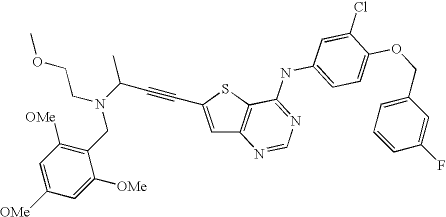

### (2R,S)—N-{3-chloro-4-[(3-fluorobenzyl)oxy]phenyl}-6-{3-[(2-methoxyethyl)(2,4,6-trimethoxybenzyl)amino]but-1-ynyl}thieno[3,2-d]pyrimidin-4-amine

6-Bromo-N-{3-chloro-4-[(3-fluorobenzyl)oxy]phenyl}thieno[3,2-d]pydmidin-4-amine (0.20 g, 0.43 mmol) was combined with (2R,S)—N-(2-methoxyethyl)N-(2,4,6-trimethoxybenzyl)but-3-yn-2-amine (0.40 g, 1.3 mmol), dichlorobis(triphenylphosphine) palladium (II) (0.03 g), copper (I) iodide (0.03 g, 0.16 mmol), and triethylamine (0.30 mL, 2.1 mmol) in 4 mL DMF. The reaction mixture was heated to 60° C. for 0.5 h, then cooled to room temperature and filtered through Celite. The filtrate was poured into 30 mL water and extracted with ethyl acetate (2×30 mL). The combined organic layers were washed with 30 mL each water and brine, and dried over sodium sulfate. The solvent was removed with a rotary evaporator and the crude product was adsorbed to silica gel. Flash chromatography eluting with 50-100% ethyl acetate in hexane gradient gave 0.10 g of the title compound as a tan solid. LC-MS: 692 (MH⁺) HPLC RT: 3.39 min.

### Step D

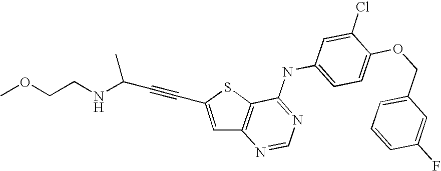

### (2R,S)—N-{3-Chloro-4-[(3-fluorobenzyl)oxy]phenyl}-6-{3-[(2-methoxyethyl)amino]but-1-ynyl}thieno[3,2-d]pyrimidin-4-amine

(2R,S)—N-{3-Chloro-4-[(3-fluorobenzyl)oxy]phenyl}-6-{3-[(2-methoxyethyl)(2,4,6-trimethoxybenzyl)amino]but-1-ynyl}thieno[3,2-d]pyrimidin-4-amine (0.09 g, 0.13 mmol) was dissolved in 0.5 mL dichloromethane and 1.0 mL trifluoroacetic acid was added. The reaction mixture was stirred for 16 h then poured into 30 mL water, where the pH was adjusted to 10 with 6 N NaOH. The aqueous mixture was extracted with chloroform (2×30 mL). The combined organic layers were washed with 30 mL brine, and dried over sodium sulfate. The solvent was removed with a rotary evaporator and the crude product was adsorbed to silica gel. Flash chromatography with 5-10% methanol in dichloromethane gave 0.031 g of the title compound as a tan solid. LC-MS: 511 (MH⁺) HPLC RT: 3.10 min.

### Example 127

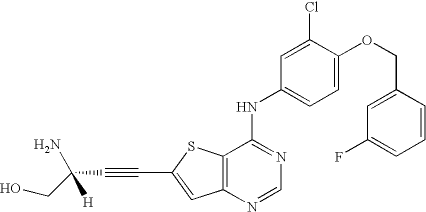

### (2R)-2-amino-4-[4-({3-chloro-4-[(3-fluorobenzyl)oxy]phenyl}amino)thieno[3,2-d]pyrimidin-6-yl]but-3-yn-1-ol

### Step A

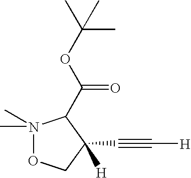

### tert-Butyl (4R)-4-ethynyl-2,2-dimethyl-1,3-oxazolidine-3-carboxylate

The title compound was prepared by combining tert-butyl (4S)-4-formyl-2,2-dimethyl-1,3-oxazolidine-3-carboxylate (Aldrich, 2.3 g, 10 mmol), with dimethyl-1-diazo-2-oxopropyl phosphonate (2.3 g, 12 mmol) and potassium carbonate (2.8 g, 20 mmol) in methanol (60 mL) and stirring at ambient temperature for 18 h. The solution was partially concentrated and diluted with ethyl acetate and extracted with water. The aqueous layer was neutralized with ammonium chloride and re-extracted with ethyl acetate. The combined organic layers were dried with MgSO4, filtered and concentrated. The resulting residue was purified by silica gel chromatography with ethyl acetate:hexanes (1:10) to provide the title compound as a clear oil (1.7 g, 77%). ¹H NMR (DMSO-d₆) δ 4.53 (br s, 1H), 4.00 (dd, 1H), 3.88 (dd, 1H), 3.21 (br s, 1H), 1.50 (s, 3H), 1.42 (s, 9H), 1.40 (s, 3H).

### Step B

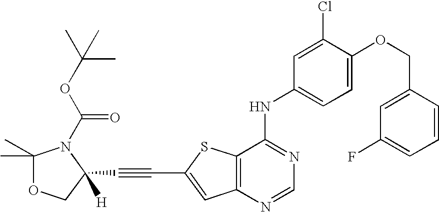

### tert-Butyl (4R)-4-{[4-({3-chloro-4[(3-fluorobenzyl)oxy]phenyl}amino)thieno[3,2-d]pyrimidin-6-yl]ethynyl}-2,2-dimethyl-1,3-oxazolidine-3-carboxylate

6-Bromo-N-{3-chloro-4-[(3-fluorobenzyl)oxy]phenyl}thieno[3,2-d]pyrimidin-4-amine hydrochloride (2.5 g, 5.0 mmol) was combined with Cul (95 mg, 0.5 mmol), dichlorobis(triphenylphosphine)palladium(II) (175 mg, 0.25 mmol), THF (50 mL), triethylamine (2.8 mL, 20 mmol), and tert-butyl (4R)-4-ethynyl-2,2-dimethyl-1,3-oxazolidine-3-carboxylate (1.4 g, 6.2 mmol). The mixture was stirred at 60 C for 4 h, filtered through Celite and concentrated. The residue was dissolved in ethyl acetate and extracted with water. The combined organic layers were dried (MgS0₄), filtered and concentrated. The residue was purified by silica gel chromatography with methanol:dichloromethane (1:100) to give the desired product as a gold solid (2.9 g, 95%). ¹H NMR (DMSO-d₆) δ 9.73 (s, 1H), 8.58 (s, 1H), 7.91 (d, 1H), 7.59 (br s, 2H), 7.51-7.43 (m, 1H), 7.33-7.15 (m, 4H), 5.25 (s, 2H), 4.90 (s, 1H), 4.15-4.05 (m, 2H), 1.57 (s, 3H), 1.45 (s, 9H), 1.42 (s, 3H). MS (ES+): 609 (MH⁺).

### Step C

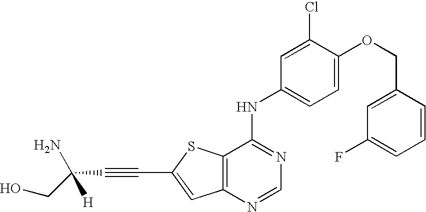

(2R)-2-amino-4-[4-({3-chloro-4-[(3-fluorobenzyl)oxy]phenyl}amino)thieno[3,2-d]pyrimidin-6-yl]but-3-yn-1-ol

The title compound was prepared from tert-butyl (4R)-4-([4-({3-chloro-4-[(3-fluorobenzyl)oxy]phenyl}amino)thieno[3,2-d]pyrimidin-6-yl]ethynyl)-2,2-dimethyl-1,3-oxazolidine-3-carboxylate (1.5 g, 2.47 mmol) by dissolving it in dichloromethane (40 mL) and adding trifluoroacetic acid (10 mL). After five hours the solution was concentrated, then reconstituted in dichloromethane (30 mL) and diisopropylethylamine on polystyrene beads (DIEA-PS) (0.4 g) was added, and stirred gently. After 2 h the resin was filtered off and the crude product was purified on a silica gel column with methanol:dichloromethane (1:20) to give the desired product as a gold solid (0.75 g, 65%). ¹H NMR (DMSO-d₆) δ 9.77 (s,1H), 8.59 (s,1H), 7.90 (d, 1H), 7.60 (dd, 2H), 7.50-7.43 (m, 1H), 7.33-7.15 (m, 4H), 5.25 (s, 2H), 3.4-3.1 (m, 3H). MS (ES+): 469 (MH⁺).

### Example 128

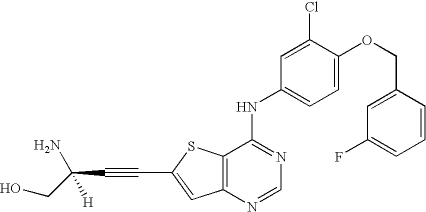

### (2S)-2-amino-4-[4-({3-chloro-4-[(3-fluorobenzyl)oxy]phenyl}amino)thieno[3,2-d]pyrimidin-6-yl]but-3-yn-1-ol

### Step A

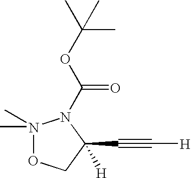

### tert-Butyl (4S)4-ethynyl-2,2-dimethyl-1,3-oxazolidine-3-carboxylate

The title compound was prepared by combining tert-butyl (4R)-4-formyl-2,2-dimethyl-1,3-oxazolidine-3-carboxylate (2.3 g, 10 mmol), with dimethyl-1-diazo-2-oxopropyl phosphonate (2.3 g, 12 mmol) and potassium carbonate (2.8 g, 20 mmol) in methanol (60 mL) and stirring atambient temperaturefor 18 h. The solution was partially concentrated and diluted with ethyl acetate and extracted with water. The aqueous layer was neutralized with ammonium chloride and re-extracted with ethyl acetate. The combined The combined organic layers were dried with MgSO₄, filtered and concentrated. The resulting residue was purified by silica gel chromatography with ethyl acetate:hexanes (1:10) to provide the title compound as a clear oil (1.6 g, 70%). ¹H NMR (DMSO-d₆) δ 4.53 (br s, 1H), 4.00 (dd, 1H), 3.88 (dd, 1H), 3.21 (br s, 1H), 1.50 (s, 3H), 1.42 (s, 9H), 1.40 (s, 3H).

### Step B

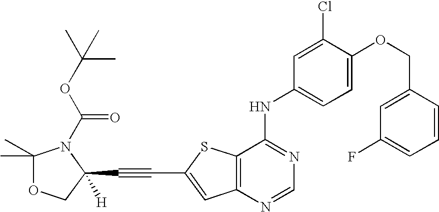

### tert-Butyl (4S)-4-{[4-({3-chloro-4-[(3-fluorobenzyl)oxy]phenyl}amino)thieno[3,2-d]pyrimidin-6-yl]ethynyl}-2,2-dimethyl-1,3-oxazolidine-3-carboxylate

6-Bromo-N-(3-chloro-4-[(3-fluorobenzyl)oxy]phenyl}thieno[3,2-d]pyrimidin4-amine hydrochloride (1.5 g, 3.0 mmol) was combined with Cul (60 mg, 0.3 mmol), dichlorobis(triphenylphosphine)palladium(II) (100 mg, 0.15 mmol), THF (40 mL), triethylamine (1.7 mL, 12 mmol), and tert-butyl (4S)H4-ethynyl-2,2-dimethyl-1,3-oxazolidine-3-carboxylate (0.72 g, 3.2 mmol). The mixture was stirred at 60° C. for 18 h, filtered through Celite and concentrated. The residue was dissolved in ethyl acetate and extracted with water and aqueous sodium bicarbonate. The combined organic layers were dried (MgSO₄), filtered, and concentrated. The residue was purified by silica gel chromatography with methanol:dichloromethane (1:100) to give the desired product as a gold solid (1.8 g , 95%). ¹H NMR (DMSO-d₆) δ 9.73 (s,1H), 8.58 (s, 1H), 7.91 (d,1H), 7.59 (br s, 2H), 7.51-7.43 (m,1H), 7.33-7.15 (m, 4H), 5.25 (s, 2H), 4.90 (s, 1H), 4.15-4.05 (m, 2H), 1.57 (s, 3H), 1.45 (s, 9H), 1.42 (s, 3H). MS (ES+): 609 (MH⁺).

### Step C

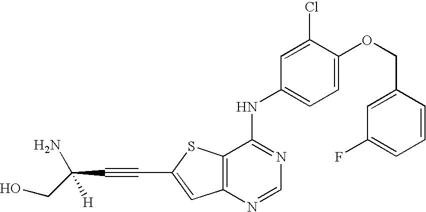

### (2S)-2-amino-4-[4-({3-chloro-4-[(3-fluorobenzyl)oxy]phenyl}amino)thieno[3,2-d]pyrimidin-6-yl]but-3-yn-1-ol

The title compound was prepared from tert-butyl (4S)-4-{[4-({3-chloro-4-[(3-fluorobenzyl)oxy]phenyl}amino)thieno[3,2-d]pyrimidin-6-yl]ethynyl}-2,2-dimethyl-1,3-oxazolidine-3-carboxylate (0.10 g, 0.16 mmol) by dissolving it in dichloromethane (4 mL) and adding trifluoroacetic acid (1 mL). After five hours the solution was concentrated, then reconstituted in dichloromethane (5 mL) and diisopropylethylamine on polystyrene beads (DIEA-PS) (0.15 g) was added, and stirred gently. After 2 h the resin was filtered off and the crude product was purified on a silica gel column with methanol: dichloromethane (1:20) to give the desired product as a gold solid (0.065 g, 87%). ¹H NMR (DMSO-d₆) δ 9.77 (s, 1H), 8.59 (s,1H), 7.90 (d, 1H), 7.60 (dd, 2H), 7.50-7.43 (m, 1H), 7.33-7.15 (m, 4H), 5.25 (s, 2H), 3.4-3.1 (m, 3H). MS (ES+): 469 (MH⁺).

Biological Data:

Compounds of the present invention were tested for ErbB family protein tyrosine kinase inhibitory activity in substrate phosphorylation assays and cell proliferation assays.

Enzyme Assays:

Compounds of the present invention were tested for EGFR, ErbB-2, and ErbB-4 protein tyrosine kinase inhibitory activity in substrate phosphorylation assays using enzymes purified from a baculovirus expression system. Reagent production and assay methodology were conducted essentially as described (Brignola, P.S., et al, (2002) J. Biol. Chem. v. 277 in press).

The method measures the ability of the isolated enzyme to catalyse the transfer of the γ-phosphate from ATP onto tyrosine residues in a biotinylated synthetic peptide (biotin-Ahx-RAHEEIYHFFFAKKK-amide). Reactions were performed in 96-well polystyrene round-bottom plates in a final volume of 45 μL. Reaction mixtures contained 50 mM MOPS (pH 7.5), 2 mM MnCl₂, 10 μM ATP, 0.125 μCi [γ-³³P] ATP per reaction, 2 μM peptide substrate, and I mM dithiothreitol. Reactions were initiated by adding 1 pmol (20 nM) per reaction of the indicated enzyme. The reaction was allowed to proceed for 15 minutes, terminated and quantified using a scintillation proximity assay procedure as described in McDonald, O. B., Antonsson, B., Arkinstal, S., Marshall, C. J., and Wood, E. R. (1999) Analytical Biochemistry, 268, 318-329.

Compounds under analysis were dissolved in Me₂SO to 0.5 mM and serially diluted 1 to 3 with Me₂SO through eleven columns of a 96 well plate. 1 μL of each concentration was transferred to the corresponding well of the assay plate. This creates a final compound concentration range from 0.00019 to 11.1 μM.

The data for dose responses were plotted as % Control calculated with the data reduction formula 100*(U1−C2)/(C1−C2) versus concentration of compound and fitted to the curve described by:
y=((Vmax*x)/(K+x))
where Vmax is the upper asymptote and K is the IC₅₀.
Cellular assays: Methylene Blue Growth Inhibition Assay

Human breast (BT474), head and neck (HN5) and gastric tumor (N87) cell lines and human foreskin Fibroblasts (HFF) were cultured in low glucose DMEM (Life Technologies 12320-032) containing 10% fetal bovine serum (FBS) at 37° C. in a humidified 10% CO₂, 90% air incubator. The SV40 transformed human mammary epithelial cell line HB4a was transfected with either human H-ras cDNA (HB4a r4.2) or the human c-ErbB2 cDNA (HB4a c5.2). The HB4a clones were cultured in RPMI containing 10% FBS, insulin (5 μg/ml), hydrocortisone (5 μg/ml), supplemented with the selection agent hygromycin B (50μg/ml). Cells were harvested using trypsin/EDTA, counted using a haemocytometer, and plated in 100 ml of the appropriate media, at the following densities, in a 96-well tissue culture plate (Falcon 3075): BT474 10,000 cells/well, HN5 3,000 cells/well, N87 10,000 cells/well, HB4a c5.2 3,000 cells/well, HB4a r4.2 3,000 cells/well, HFF 2500 cells/well. The next day, compounds were diluted in DMEM containing 100 mg/ml gentamicin, at twice the final required concentration, from 10 mM stock solutions in DMSO. 100 ml/well of these dilutions were added to the 100 ml of media currently on the cell plates. Medium containing 0.6% DMSO was added to control wells. Compounds diluted in DMEM were added to all cell lines, including the HB4a r4.2 and HB4a c5.2 cell lines. The final concentration of DMSO in all wells was 0.3%. Cells were incubated at 37° C., 10% CO2 for 3 days. Medium was removed by aspiration. Cell biomass was estimated by staining cells with 100 μl per well methylene blue (Sigma M9140, 0.5% in 50:50 ethanol:water), and incubation at room temperature for at least 30 minutes. Stain was removed, and the plates rinsed under a gentle stream of water, and air-dried. To release stain from the cells 100 μl of solubilization solution was added (1% N-lauroyl sarcosine, Sodium salt, Sigma L5125, in PBS), and plates were shaken gently for about 30 minutes. Optical density at 620 nM was measured on a microplate reader. Percent inhibition of cell growth was calculated relative to vehicle treated control wells. Concentration of compound that inhibits 50% of cell growth (IC₅₀) was interpolated using nonlinear regression (Levenberg-Marquardt) and the equation, y=Vₘₐₓ*(1−(x/(K+x)))+Y2, where “K” was equal to the IC₅₀.

Table I illustrates the inhibitory activity of compounds of the present invention as IC₅₀ values in μM against and the BT474 tumor cell line. Using HFF as a representative, human, normal cell line, values for cytotoxicity are supplied as IC50 values in micromolar.

TABLE I

| | Enzyme | Cell |
|---|---|---|
| Compound name | Activity | Activity |
| N-{3-chloro-4-[(3-fluorobenzyl)oxy]phenyl}-6-[3- | + | + |
| (diisobutylamino)prop-1-ynyl]thieno[3,2-d]pyrimidin-4-amine | | |
| N-{3-[4-({3-chloro-4-[(3- | + | + |
| fluorobenzyl)oxy]phenyl}amino)thieno[3,2-d]pyrimidin-6- | | |
| yl]prop-2-ynyl}-2-(3,4-dichlorophenyl)acetamide | | |
| N-{3-[4-({3-chloro-4-[(3- | ++ | ++ |
| fluorobenzyl)oxy]phenyl}amino)thieno[3,2-d]pyrimidin-6- | | |
| yl]prop-2-ynyl}-2-pyridin-4-yl-1,3-thiazole-4-carboxamide | | |
| N-{3-[4-({3-chloro-4-[(3- | ++ | ++ |
| fluorobenzyl)oxy]phenyl}amino)thieno[3,2-d]pyrimidin-6- | | |
| yl]prop-2-ynyl}-3-(4-fluorophenyl)propanamide | | |
| 2,6-dichloro-N-{3-[4-({3-chloro-4-[(3- | ++ | ++ |
| fluorobenzyl)oxy]phenyl}amino)thieno[3,2-d]pyrimidin-6- | | |
| yl]prop-2-ynyl}benzamide | | |
| N-{3-[4-({3-chloro-4-[(3- | ++ | ++ |
| fluorobenzyl)oxy]phenyl}amino)thieno[3,2-d]pyrimidin-6- | | |
| yl]prop-2-ynyl}-1H-indole-5-carboxamide | | |
| N-{3-chloro-4-[(3-fluorobenzyl)oxy]phenyl}-6-(pyrimidin-2- | ++ | ++ |
| ylethynyl)thieno[2,3-d]pyrimidin-4-amine | | |
| N-{3-[4-({3-chloro-4-[(3- | +++ | +++ |
| fluorobenzyl)oxy]phenyl}amino)thieno[3,2-d]pyrimidin-6- | | |
| yl]prop-2-ynyl}-2-(4-methylpiperazin-1-yl)acetamide | | |
| N-{3-chloro-4-[(3-fluorobenzyl)oxy]phenyl}-6-(3-{[2- | +++ | +++ |
| (methylsulfonyl)ethyl]amino}prop-1-ynyl)thieno[3,2- | | |
| d]pyrimidin-4-amine hydrochloride | | |
| N-{3-[4-({3-chloro-4-[(3- | +++ | +++ |
| fluorobenzyl)oxy]phenyl}amino)thieno[2,3-d]pyrimidin-6- | | |
| yl]prop-2-ynyl}-N′-[2-(methylsulfonyl)ethyl]urea | | |
| N-{3-[4-({3-chloro-4-[(3- | +++ | +++ |
| fluorobenzyl)oxy]phenyl}amino)thieno[2,3-d]pyrimidin-6-yl]prop-2- | | |
| ynyl}-N′-[2-(dimethylamino)ethyl]urea | | |

+ Enzyme (ErbB-2) < 7.0 pIC50. Cell (BT474) > 5 μM
++ Enzyme 7.0 < pIC50 < 7.5. Cell < 5 μM, > 1.5 μM
+++ Enzyme > 7.5 pIC50, Cell <1.5 μM

1. A compound of formula (I), or salts thereof, 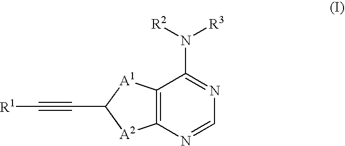
wherein:
one of A¹ and A² is S and the other is CH;
R¹ is H or —(CR¹¹ R ¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂),heteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R ⁶)—C(S)—N(R⁶)(R⁷), —N(R⁶)—C(O)—OR⁷, —N(R⁶)—C(O)—(CH₂)ₙ—R⁷, —N(R⁶)—SO₂R⁶, —(CH₂)ₙNR⁶R⁷, —(CH₂)ₙOR⁷ —(CH₂)ₙSR⁸, —(CH₂)ₙS(O) R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, —(CH₂)ₙS(O)₂R⁸, —(CH₂)ₙR⁸, —CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

2. A compound according to claim 1, wherein:
one of A¹ and A² is S and the other is CH;
R¹ is —(CR¹¹R¹¹)ₙ—R⁵;
R² is H;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂),heteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, (CH₂)ₙS(O)₂R⁸, —(CH₂)ₙR^{8,} and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

3. A compound according to claim 1, wherein:
one of A¹ and A² is S and the other is CH;
R¹ is —(CR¹¹R¹¹)ₙ—R⁵;
R²is H;
R³is aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂),aryl and —(CH₂)ₙNR⁹R¹⁰;
R⁴is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

4. A compound according to claim 1, wherein:
one of A¹ and A² is S and the other is CH;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸,—(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

5. A compound according to claim 1, wherein:
A¹ is S and A² is CH;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and
—(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, ≦CN, —SO₂R⁹, and —(CH₂)ₙNR⁹R¹⁰, a (CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R⁶)—C(S)—N(R⁶)(R⁷), —N(R⁶)—C(O)—OR⁷, —N(R⁶)—C(O)—(CH₂)ₙ—R⁷, —N(R⁶)—SO₂R⁸, ——(CH₂)ₙN R⁶R⁷, —(CH₂)ₙOR⁷, —(CH₂)ₙSR⁸, —(CH₂)ₙS(O) R⁸, —(CH₂)ₙS(O)₂R⁸, —OC(O)R⁸, —OC(O)OR⁸, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R^{9,} and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁶ and R⁷ are independently selected from the group consisting of H, C₁₋₆alkyl, C₃₋₈cycloalkyl, heterocyclyl, (CH₂)ₙNR⁹R¹⁰, —(CH₂)ₙOR⁹, —(CH₂)ₙ—C(O)R⁸, —C(O)₂R⁸, —(CH₂)ₙSR⁸, —(CH₂)ₙS(O)R⁸, —(CH₂)ₙS(O)₂R⁸, —(CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

6. A compound according to claim 1, wherein:
A¹ is CH and A² is S;
R¹ is H or —(CR¹¹R¹¹)ₙ—R⁵;
R² is H or C₁₋₆alkyl;
R³ is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆ alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹,—(CH₂)ₙaryl and —(CH₂)ₙNR⁹R¹⁰;
R⁴ is selected from the group consisting of H, C₁₋₆alkyl, —(CH₂)ₙNR⁹R¹⁰, —(CH₂)ₙheterocyclyl, —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R^{9,} and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of heterocyclyl, —N(R⁶)—C(O)—N(R⁶)(R⁷), —N(R⁶)—C(S)—N(R⁶)(R⁷), —N(R⁶)—C(O)—OR⁷, —N(R⁶)—C(O)—(CH₂)ₙ—R⁷, —N(R⁶)—SO₂R⁶, —(CH₂)ₙNR⁶R⁷₃ —(CH₂)ₙOR⁷, —(CH₂)ₙSR⁸, —(CH₂)ₙS(O)R⁸, —(CH₂)—S(O)₂R⁸, —OC(O)R⁸, —OC(O)OR⁶, —C(O)NR⁶R⁷, heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and aryl optionally substituted with one or more substituents selected from the group consisting of halo, —CF₃, C₁₋₆alkoxy, —NO₂, C₁₋₆alkyl, —CN, —SO₂R^{9,} and —(CH₂)ₙNR⁹R¹⁰;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙC(O)R⁸, —C(O)₂R⁸, —(CH₂)ₙSR⁸, —(CH₂)ₙS(O)R⁸, (CH₂)ₙS(O)₂R⁸, (CH₂)ₙR⁸, —(CH₂)ₙCN, aryl optionally substituted with one or more substituents selected from the group consisting of halo, —CF₃, C₁₋₆alkoxy, —NO₂, C₁₋₆alkyl, —CN, —(CH₂)ₙOR⁸, —(CH₂)ₙheterocyclyl, —(CH₂)ₙheteroaryl, —SO₂R⁹, and —(CH₂)ₙNR⁹¹⁰, and heteroaryl optionally substituted with one or more substituents selected from the group consisting of halo, —CF₃, C₁₋₆alkoxy, —NO₂, C₁₋₆alkyl, —CN, —(CH₂)ₙOR⁸ —(CH₂)ₙheterocyclyl, —(CH₂)ₙheteroaryl, —SO₂R⁹, and —(CH₂)ₙNR⁹R¹⁰, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H, C₁₋₆alkyl, C₃₋₈cycloalkyl, and —C(O)R¹¹ or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

7. A compound according to claim 1, wherein:
A¹ is S and A² is CH;
R¹ is —(CR¹¹R¹¹)ₙ—R⁵;
R is H;
R³ is selected from the group consisting of aryl optionally substituted with one or more substituents selected from the group consisting of halo, alkynyl, —CF₃, —(CH₂)ₙOR⁴, —(CH₂)—SR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

8. A compound according to claim 1, wherein:
A¹ is CH and A² is S;
R¹ is —(CR¹¹R¹¹)ₙ—R⁵;
R² is H;
R³is selected from the group consisting of aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰, and heteroaryl optionally substituted with one or more substituents selected from the group consisting of halo, alkynyl, —CF₃, (CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, arylC₁₋₆alkenylene in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkenylene in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of
N(R⁶)—C(O)—N(R⁶)(R⁷), —N(R⁶)—C(O)—(CH₂)ₙ—R⁷, and —(CH₂)ₙNR ⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

9. A compound according to claim 1, wherein:
A¹ is S and A² is CH;
R¹ is —(CR¹¹R¹¹)ₙ—R⁵;
R² is H;
R³ is aryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

10. A compound according to claim 1, wherein:
A¹ CH and A² is S;
R¹ is —(CR¹¹R¹¹)ₙ—R⁵;
R² is H;
R³ is aryl optionally substituted with one or more substituents selected from the group consisting of halo, alkynyl, —CF₃, —(CH₂)ₙOR⁴, —(CH₂)ₙSR⁴, NO₂, C₁₋₆alkyl, —CN, —SO₂R⁹, —(CH₂)ₙaryl and —(CH₂)ₙNR⁹R¹⁰;
R⁴is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)—S(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

11. A compound according to claim 1, wherein:
A¹ is S and A² is CH;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂),aryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R^{9,} and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, (CH₂)ₙR⁸, and —(CH₂)ₙCN, or R and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C_{1 6}alkyl, and C₃₋₈cycloalkyl; and
n is 0-6.

12. A compound according to claim 1, wherein:
A¹ is CH and A² is S;
R¹ is —(CR¹¹R¹¹)ₙ—R⁵;
R² is H;
R³ is heteroaryl optionally substituted with one or more substituents selected from the group consisting of halo, alkynyl, —CF₃, —(CH₂)ₙOR⁴, —(CH₂)ₙSR⁴, —NO₂, C₁₋₆alkyl, —CN, —SO₂R⁹, —(CH₂)ₙaryl and —(CH₂)ₙNR⁹R¹⁰;
R⁴ is selected from the group consisting of —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and —(CH₂)ₙheteroaryl in which heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, (CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO_{2,} C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

13. A compound according to claim 1, wherein:
A¹ is S and A² is CH;
R¹ is —(CR¹¹R¹¹)ₙ—R⁵;
R² is H;
R³ is aryl optionally substituted with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(C H₂)ₙS(O)₂R⁸, and —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆, alkyl, or R⁹ and R¹⁰, together with the atom to which they are attached, form a 3-8 membered ring;
R¹¹ is independently selected from the group consisting of H, C₁ 6alkyl, and C₃₋₈cycloalkyl; and
n is 0-6.

14. A compound according to claim 1, wherein:
A¹ is S and A² is CH;
R¹ is —(CR¹¹R¹¹)ₙ—R⁵;
R² is H;
R³ is aryl optionally substituted in the para position with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙ; NR⁹R¹⁰, —(CH₂)ₙOR⁹, —(CH₂)ₙS(O)₂R⁸, —(CH₂)ₙR⁸, and —(CH₂)ₙCN, or R⁶ and R⁷, together with the atom to which they are attached, form a 3-8 membered ring;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

15. A compound according to claim 1, wherein:
A¹ is S and A² is CH;
R¹ is —(CR¹¹R¹¹)ₙ—R⁵, n=0-6;
R² is H;
R³ is aryl optionally substituted in the para position with —(CH₂)ₙOR⁴, and in the meta position with halogen, —CN, C₁₋₆alkyl, or alkynyl;
R⁴ is —(CH₂)ₙaryl in which aryl is optionally substituted with halo;
R⁵ is selected from the group consisting of —N(R⁶)—C(O)—N(R⁶)(R⁷), —N(R⁶)—C(O)—(CH₂)ₙ—R⁷, and —(CH₂)ₙNR⁶R⁷;
R⁶ and R⁷ are independently selected from the group consisting of H, C₁₋₆alkyl, heterocyclyl, —(CH₂)ₙNR⁹¹⁰R, —(CH₂)ₙOR⁹, —(CH₂)ₙS(O)₂R⁸, and —(CH₂)ₙCN, or R⁶ and R⁷, together with the atom to which they are attached, form a 3-8 membered ring, n=0-6;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n=0-6.

16. A compound according to claim 1, wherein:
A¹ CH and A² is S;
R¹ is —(CR¹¹R¹¹)ₙ—R⁵;
R² is H;
R³ is aryl optionally substituted in the para position with —(CH₂)ₙOR⁴ and in the meta position with halogen, —CN, C₁₋₆alkyl, or alkynyl;
R⁴is —(CH₂)ₙaryl in which aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁵ is —N(R⁶)—C(O)—N(R⁶)(R⁷) —N(R⁶)—C(O)—(CH₂)ₙ—R⁷ or —(CH₂)ₙNR⁶R⁷;
R⁶ and R⁷ are independently selected from the group consisting of H, C₁₋₆alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

17. A compound according to claim 1, wherein:
A¹ CH and A² is S;
R¹ is —(CR¹¹R¹¹)ₙ—R⁵;
R² is H;
R³ is aryl substituted in the para position with —(CH₂)ₙOR⁴ and in the meta position with halogen, —CN, C₁₋₆alkyl, or alkynyl;
R⁴ is —(CH₂)ₙaryl in which aryl is optionally substituted with halo;
R⁵ is —N(R⁶)—C(O)—N(R⁶)(R⁷), —N(R⁶)—C(O)—(CH₂)ₙ—R⁷, or —(CH₂)ₙNR⁶R⁷;
R⁶ and R⁷ are independently selected from the group consisting of H, C₁₋₆alkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n is 0-6.

18. A compound according to claim 1, wherein:
A¹ CH and A² is S;
R¹ is —(CR¹¹R¹¹)ₙ—R⁵;
R² is H;
R³ is aryl substituted in the para position with —(CH₂)ₙOR⁴ and in the meta position with halogen, —CN, C₁₋₆alkyl, or alkynyl;
R⁴ is —(CH₂)ₙaryl substituted with halo;
R⁵ is —N(R⁶)—C(O)—N(R⁶)(R⁷), —N(R⁶)—C(O)—(CH₂)ₙ—R⁷ or —(CH₂)ₙNR⁶R⁷;
R⁶ and R⁷ are independently selected from the group consisting of H, C¹⁻⁶alkyl, C₃₋₈cycloalkyl, heterocyclyl, —(CH₂)ₙNR⁹R¹⁰, —(CH₂)ₙOR⁹, —(CH₂)ₙS(O)₂R⁸, —(CH₂)ₙR⁸, and —(CH₂)ₙCN;
R⁸ is selected from the group consisting of C₁₋₆alkyl, C₃₋₈cycloalkyl, heterocyclylC₁₋₆alkylene, arylC₁₋₆alkylene wherein said aryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, heteroarylC₁₋₆alkylene wherein said heteroaryl is optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, aryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰, and heteroaryl optionally substituted with one or more substituents selected from the group consisting of halo, —CF₃, C₁₋₆alkoxy, —NO₂, C₁₋₆alkyl, —CN, —SO₂R⁹, and —(CH₂)ₙNR⁹R¹⁰;
R⁹ and R¹⁰ are independently selected from the group consisting of H and C₁₋₆alkyl, or R⁹ and R¹⁰, together with the atom to which they are attached, form a 3-8 membered ring;
R¹¹ is independently selected from the group consisting of H, C₁₋₆alkyl, and C₃₋₈cycloalkyl; and
n=0-6.

19. A compound selected from the group consisting of:
N-(2-benzyl-1H-benzimidazol-5-yl)-6-ethynylthieno[3,2-d]pyrimidin-4-amine;
N-(2-benzyl-1H-benzimidazol-5-yl)-6-(1H-pyrazol-4-ylethynyl)thieno[3,2-d]pyrimidin-4-amine;
N-{3-chloro-4-[(3-fluorobenzyl)oxy]phenyl}-6-ethynylthieno[3,2-d]pyrimidin-4-amine;
N-(2-benzyl-1H-benzimidazol-5-yl)-6-[3-(1,1-dioxidothiomorpholin-4-yl)prop-1-ynyl]thieno[3,2-d]pyrimidin-4-amine;
N-{3-chloro-4-[(3-fluorobenzyl)oxy]phenyl}-6-[3-(1,1-dioxidothiomorpholin-4-yl)prop-1-ynyl]thieno[3,2-d]pyrimidin-4-amine hydrochloride;
N-(1-benzyl-1H-indazol-5-yl)-6-[3-(1,1-dioxidothiomorpholin-4-yl)prop-1-ynyl]thieno[3,2-d]pyrimidin-4-amine hydrochloride;
N-(1-benzyl-1H-indazol-5-yl)-6-ethynylthieno[2,3-d]pyrimidin-4-amine;
N-(2-benzyl-1H-benzimidazol-6-yl)-6-ethynylthieno[2,3-d]pyrimidin-4-amine;
N-{3-chloro-4-[(3-fluorobenzyl)oxy]phenyl}-6-ethynylthieno[2,3-d]pyrimidin-4-amine;
N-(1-benzyl-1H-indazol-5-yl)-6-(3-morpholin-4-ylprop-1-ynyl)thieno[3,2-d]pyrimidin-4-amine hydrochloride;
N-(2-benzyl-1H-benzimidazol-5-yl)-6-(3-morpholin-4-ylprop-1-ynyl)thieno[3,2-d]pyrimidin-4-amine;
N-{3-chloro-4-[(3-fluorobenzyl)oxy]phenyl}-6-(3-morpholin-4-ylprop-1-ynyl)thieno[3,2-d]pyrimidin-4-amine hydrochloride;
N-(1-benzyl-1H-benzimidazol-5-yl)-6-(3-morpholin-4-ylprop-1-ynyl)thieno[3,2-d]pyrimidin-4-amine hydrochloride;
N-{3-[4-({3-chloro-4-[(3-fluorobenzyl)oxy]phenyl}amino)thieno[3,2-d]pyrimidin-6-yl]prop-2-ynyl}urea hydrochloride;
N-(3-{4-[(1-benzyl-1H-indazol-5-yl)amino]thieno[3,2-d]pyrimidin-6-yl}prop-2-ynyl)urea hydrochloride;
N-(3-{4-[(2-benzyl-1,3-benzoxazol-6-yl)amino]thieno[3,2-d]pyrimidin-6-yl}prop-2-ynyl)urea hydrochloride;
N-(2-benzyl-1,3-benzoxazol-6-yl)-6-ethynylthieno[3,2-d]pyrimidin-4-amine hydrochloride;
N-(1-benzyl-1H-indol-5-yl)-6-ethynylthieno[3,2-d]pyrimidin-4-amine hydrochloride;
N-(2-benzyl-1-benzofuran-5-yl)-6-ethynylthieno[3,2-d]pyrimidin-4-amine hydrochloride;
N-(3-{4-[(2-benzyl-1H-benzimidazol-5-yl)amino]thieno[3,2-d]pyrimidin-6-yl}prop-2-ynyl)urea hydrochloride;
N-(2-benzyl-1,3-benzothiazol-6-yl)-6-ethynylthieno[3,2-d]pyrimidin-4-amine hydrochloride;
N-(2-benzyl-1,3-benzothiazol-5-yl)-6-ethynylthieno[3,2-d]pyrimidin-4-amine hydrochloride;
N-(4-benzylphenyl)-6-ethynylthieno[3,2-d]pyrimidin-4-amine;
6-ethynyl-N-[4-(1-naphthyloxy)phenyl]thieno[3,2-d]pyrimidin-4-amine hydrochloride;
6-ethynyl-N-[4-(3-methoxyphenoxy)phenyl]thieno[3,2-d]pyrimidin-4-amine hydrochloride;
6-ethynyl-N-[4-(4-methylphenoxy)phenyl]thieno[3,2-d]pyrimidin-4-amine hydrochloride;
6-ethynyl-N-[4-(4-methylphenoxy)phenyl]thieno[3,2-d]pyrimidin-4-amine;
N-{3-chloro-4-[(3-fluorobenzyl)oxy]phenyl}-6-[3-(dimethylamino)prop-1-ynyl]thieno[3,2-d]pyrimidin-4-amine hydrochloride;
N-(3-{[4-({3-chloro-4-[(3-fluorobenzyl)oxy]phenyl}amino)thieno[3,2-d]pyrimidin-6-yl]ethynyl}phenyl)acetamide hydrochloride;
N-[3-({4-[(1-benzyl-1H-indazol-5-yl)amino]thieno[3,2-d]pyrimidin-6-yl}ethynyl)phenyl]acetamide hydrochloride;
N-[3-({4-[(2-benzyl-1H-benzimidazol-5-yl)amino]thieno[3,2-d]pyrimidin-6-yl}ethynyl)phenyl]acetamide hydrochloride;
tert-butyl 3-[4-({3-chloro-4-[(3-fluorobenzyl)oxy]phenyl}amino)thieno[3,2-d]pyrimidin-6-yl]prop-2-ynylcarbamate hydrochloride;
tert-butyl 3-{4-[(1-benzyl-1H-indazol-5-yl)amino]thieno[3,2-d]pyrimidin-6-yl}prop-2-ynylcarbamate hydrochloride;
N-{3-[4-({3-chloro-4-[(3-fluorobenzyl)oxy]phenyl}amino)thieno[3,2-d]pyrimidin-6-yl]prop-2-ynyl}acetamide hydrochloride;
N-(3-{4-[(1-benzyl-1H-indazol-5-yl)amino]thieno[3,2-d]pyrimidin-6-yl}prop-2-ynyl)acetamide hydrochloride;
N-(3-{4-[(2-benzyl-1H-benzimidazol-5-yl)amino]thieno[3,2-d]pyrimidin-6-yl}prop-2-ynyl)acetamide hydrochloride;
6-ethynyl-N-(4-{[3-(trifluoromethyl)phenyl]thiophenyl)thieno[3,2-d]pyrimidin-4-amine;
6-ethynyl-N-[2-(3-methoxybenzyl)-1H-benzimidazol-5-yl]thieno[3,2-d]pyrimidin-4-amine;
6-(3-aminoprop-1-ynyl)-N-(1-benzyl-1H-indazol-5-yl)thieno[3,2-d]pyrimidin-4-amine;
6-(3-aminoprop-1-ynyl)-N-{3-chloro-4-[(3-fluorobenzyl)oxy]phenyl}thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-N′-methylurea;
N-{3-[4-({3-chloro-4-[(3-fluorobenzyl)oxy]phenyl}amino)thieno[3,2-d]pyrimidin-6-yl]prop-2-ynyl}-N′-cyclopentylurea;
N-{3-[4-({3-chloro-4-[(3-fluorobenzyl)oxy]phenyl}amino)thieno[3,2-d]pyrimidin-6-yl]prop-2-ynyl}-4-methyibenzenesulfonamide;
N-{3-[4-({3-chloro-4-[(3-fluorobenzyl)oxy]phenyl}amino)thieno[3,2-d]pyrimidin-6-yl]prop-2-ynyl}-N′-phenylurea;
N-{3-[4-({3-chloro-4-[(3-fluorobenzyl)oxy]phenyl}amino)thieno[3,2-d]pyrimidin-6-yl]prop-2-ynyl}-2-(4-methylpiperazin-1-yl)acetamide;
N-{3-[4-({3-chloro-4-[(3-fluorobenzyl)oxy]phenyl}amino)thieno[3,2-d]pyrimidin-6-yl]prop-2-ynyl}-4-(morpholin-4-ylmethyl)benzamide;
N-{3-[4-({3-chloro-4-[(3-fluorobenzyl)oxy]phenyl}amino)thieno[3,2-d]pyrimidin-6-yl]prop-2-ynyl}-2-(1-methyl-1H-imidazol-4-yl)acetamide;
N-{3-chloro-4-[(3-fluorobenzyl)oxy]phenyl}-6-(3-{[2-(methylsulfonyl)ethyl]amino}prop-1-ynyl)thieno[3,2-d]pyrimidin-4-amine hydrochloride;
N-{3-[4-({3-chloro-4-[(3-fluorobenzyl)oxy]phenyl}amino)thieno[3,2-d]pyrimidin-6-yl]prop-2-ynyl}-4-[(4-methylpiperazin-1-yl)methyl]benzamide;
N-{3-[4-({3-chloro-4-[(3-fluorobenzyl)oxy]phenyl}amino)thieno[3,2-d]pyrimidin-6-yl]prop-2-ynyl}-4-[(dimethylamino)methyl]benzamide;
N-{3-[4-({3-chloro-4-[(3-fluorobenzyl)oxy]phenyl}amino)thieno[3,2-d]pyrimidin-6-yl]prop-2-ynyl}-4-(1H-imidazol-1-ylmethyl)benzamide;
N˜1˜-{3-[4-({3-chloro-4-[(3-fluorobenzyl)oxy]phenyl}amino)thieno[3,2-d]pyrimidin-6-yl]prop-2-ynyl}-N˜2˜,N˜2˜-dimethylglycinamide;
N-{3-[4-({3-chloro-4-[(3-fluorobenzyl)oxy]phenyl}amino)thieno[3,2-d]pyrimidin-6-yl]prop-2-ynyl}-2-pyridin-3-ylacetamide;
N-{3-[4-({3-chloro-4-[(3-fluorobenzyl)oxy]phenyl}amino)thieno[3,2-d]pyrimidin-6-yl]prop-2-ynyl}-2-pyridin-4-ylacetamide;
N-[({3-[4-({3-chloro-4-[(3-fluorobenzyl)oxy]phenyl}amino)thieno[3,2-d]pyrimidin-6-yl]prop-2-ynyl}amino)carbonyl]-4-methylbenzenesulfonamide;
N-(3-{4-[(3-chloro-4-fluorophenyl)amino]thieno[3,2-d]pyrimidin-6-yl}prop-2-ynyl)urea hydrochloride;
N-{3-[4-({3-chloro-4-[(3-fluorobenzyl)oxy]phenyl}amino)thieno[3,2-d]pyrimidin-6-yl]prop-2-ynyl}-4-methylpiperazine-1-carboxamide;
N′-{3-[4-({3-chloro-4-[(3-fluorobenzyl)oxy]phenyl}amino)thieno[3,2-d]pyrimidin-6-yl]prop-2-ynyl}-N,N-dimethylurea;
N-{3-[4-({3-chloro-4-[(3-fluorobenzyl)oxy]phenyl}amino)thieno[3,2-d]pyrimidin-6-yl]prop-2-ynyl}-4-(methylsulfonyl)benzenesulfonamide;
N˜1˜-{3-[4-({3-chloro-4-[(3-fluorobenzyl)oxy]phenyl}amino)thieno[3,2-d]pyrimidin-6-yl]prop-2-ynyl}-N˜4˜-phenylsuccinamide;
N-{3-[4-({3-chloro-4-[(3-fluorobenzyl)oxy]phenyl}amino)thieno[3,2-d]pyrimidin-6-yl]prop-2-ynyl}-5-nitro-1H-pyrrole-3-carboxamide;
N-{3-[4-({3-chloro-4-[(3-fluorobenzyl)oxy]phenyl}amino)thieno[3,2-d]pyrimidin-6-yl]prop-2-ynyl}-2-pyridin-4-yl-1,3-thiazole-4-carboxamide;
N-{3-[4-({3-chloro-4-[(3-fluorobenzyl)oxy]phenyl}amino)thieno[3,2-d]pyrimidin-6-yl]prop-2-ynyl}-1,3-benzothiazole-6-carboxamide;
N-{3-[4-({3-chloro-4-[(3-fluorobenzyl)oxy]phenyl}amino)thieno[3,2-d]pyrimidin-6-yl]prop-2-ynyl}-2,2,3,3-tetramethylcyclopropanecarboxamide;
N-{3-[4-({3-chloro-4-[(3-fluorobenzyl)oxy]phenyl}amino)thieno[3,2-d]pyrimidin-6-yl]prop-2-ynyl}-3-(4-fluorophenyl)propanamide;
N-{3-[4-({3-chloro-4-[(3-fluorobenzyl)oxy]phenyl}amino)thieno[3,2-d]pyrimidin-6-yl]prop-2-ynyl}-2-(methylsulfonyl)acetamide;
N-{3-[4-({3-chloro-4-[(3-fluorobenzyl)oxy]phenyl}amino)thieno[3,2-d]pyrimidin-6-yl]prop-2-ynyl}-2-thien-3-ylacetamide;
2,6-dichloro-N-{3-[4-({3-chloro-4-[(3-fluorobenzyl)oxy]phenyl}amino)thieno[3,2-d]pyrimidin-6-yl]prop-2-ynyl}benzamide;
N-{3-[4-({3-chloro-4-[(3-fluorobenzyl)oxy]phenyl}amino)thieno[3,2-d]pyrimidin-6-yl]prop-2-ynyl}-1H-indole-5-carboxamide;
N-{3-[4-({3-chloro-4-[(3-fluorobenzyl)oxy]phenyl}amino)thieno[3,2-d]pyrimidin-6-yl]prop-2-ynyl}-2-tetrahydro-2H-pyran-4-ylacetamide;
4-(benzyloxy)-N-{3-[4-({3-chloro-4-[(3-fluorobenzyl)oxy]phenyl}amino)thieno[3,2-d]pyrimidin-6-yl]prop-2-ynyl}benzamide;
N-{3-[4-({3-chloro-4-[(3-fluorobenzyl)oxy]phenyl}amino)thieno[3,2-d]pyrimidin-6-yl]prop-2-ynyl}-2-pyridin-2-ylacetamide;
N-{3-[4-({3-chloro-4-[(3-fluorobenzyl)oxy]phenyl}amino)thieno[3,2-d]pyrimidin-6-yl]prop-2-ynyl}-2-(2-furyl)acetamide;
N-{3-[4-({3-chloro-4-[(3-fluorobenzyl)oxy]phenyl}amino)thieno[3,2-d]pyrimidin-6-yl]prop-2-ynyl}isonicotinamide;
N-{3-[4-({3-chloro-4-[(3-fluorobenzyl)oxy]phenyl}amino)thieno[3,2-d]pyrimidin-6-yl]prop-2-ynyl}quinoline-2-carboxamide;
N′-{3-[4-({3-chloro-4-[(3-fluorobenzyl)oxy]phenyl}amino)thieno[3,2-d]pyrimidin-6-yl]prop-2-ynyl}-N,N-diisopropylurea;
N-{3-[4-({3-chloro-4-[(3-fluorobenzyl)oxy]phenyl}amino)thieno[3,2-d]pyrimidin-6-yl]prop-2-ynyl}-1-methyl-1H-pyrrole-2-carboxamide;
N-{3-[4-({3-chloro-4-[(3-fluorobenzyl)oxy]phenyl}amino)thieno[3,2-d]pyrimidin-6-yl]prop-2-ynyl}cinnoline-4-carboxamide;
2-(benzyloxy)-N-{3-[4-({3-chloro-4-[(3-fluorobenzyl)oxy]phenyl}amino)thieno[3,2-d]pyrimidin-6-yl]prop-2-ynyl}acetamide;
(2E)-N-{3-[4-({3-chloro-4-[(3-fluorobenzyl)oxy]phenyl}amino)thieno[3,2-d]pyrimidin-6-yl]prop-2-ynyl}-3-(4-methylphenyl)prop-2-enamide;
N-{3-[4-({3-chloro-4-[(3-fluorobenzyl)oxy]phenyl}amino)thieno[3,2-d]pyrimidin-6-yl]prop-2-ynyl}-1H-indazole-3-carboxamide;
(4R)-N-{3-[4-({3-chloro-4-[(3-fluorobenzyl)oxy]phenyl}amino)thieno[3,2-d]pyrimidin-6-yl]prop-2-ynyl}-2-oxo-1,3-thiazolidine-4-carboxamide;
N-{3-[4-({3-chloro-4-[(3-fluorobenzyl)oxy]phenyl}amino)thieno[3,2-d]pyrimidin-6-yl]prop-2-ynyl}-4-(dimethylamino)butanamide;
N-{3-[4-({3-chloro-4-[(3-fluorobenzyl)oxy]phenyl}amino)thieno[3,2-d]pyrimidin-6-yl]prop-2-ynyl}-4-(1H-indol-3-yl)butanamide;
N-{3-[4-({3-chloro-4-[(3-fluorobenzyl)oxy]phenyl}amino)thieno[3,2-d]pyrimidin-6-yl]prop-2-ynyl}morpholine-4-carboxamide;
N-{3-[4-({3-chloro-4-[(3-fluorobenzyl)oxy]phenyl}amino)thieno[3,2-d]pyrimidin-6-yl]prop-2-ynyl)-N′-[2-(methylsulfonyl)ethyl]urea;
N-{3-[4-({3-chloro-4-[(3-fluorobenzyl)oxy]phenyl}amino)thieno[3,2-d]pyrimidin-6-yl]prop-2-ynyl}-N′-(2-morpholin-4-ylethyl)urea;
N-{3-[4-({3-chloro-4-[(3-fluorobenzyl)oxy]phenyl}amino)thieno[3,2-d]pyrimidin-6-yl]prop-2-ynyl}-N′-[2-(dimethylamino)ethyl]urea;
3-[4-({3-chloro-4-[(3-fluorobenzyl)oxy]phenylamino)thieno[3,2-d]pyrimidin-6-yl]prop-2-yn-1-ol hydrochloride;
N-{3-[4-({3-chloro-4-[(3-fluorobenzyl)oxy]phenyl}amino)thieno[3,2-d]pyrimidin-6-yl]prop-2-ynyl}-N′-(2-cyanoethyl)urea;
tert-butyl-3-[4-({3-chloro-4-[(3-fluorobenzyl)oxy]phenyl}amino)thieno[3,2-d]pyrimidin-6-yl]prop-2-ynyl(methyl)carbamate;
N-{3-chloro-4-[(3-fluorobenzyl)oxy]phenyl}-6-[3-(methylamino)prop-1-ynyl]thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-2-(1H-indol-3-yl)acetamide;
N-{3-[4-({3-chloro-4-[(3-fluorobenzyl)oxy]phenyl}amino)thieno[3,2-d]pyrimidin-6-yl]prop-2-ynyl}-2-(3,4-dichlorophenyl)acetamide;
N-{3-[4-({3-chloro-4-[(3-fluorobenzyl)oxy]phenyl}amino)thieno[3,2-d]pyrimidin-6-yl]prop-2-ynyl}-2-(4-iodophenyl)acetamide;
6-(3-aminoprop-1-ynyl)-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-[3-(dipropylamino)prop-1-ynyl]thieno[3,2-d]pyrimidin-4-amine;
N-{3-chloro-4-[(3-fluorobenzyl)oxy]phenyl}-6-[3-(diethylamino)prop-1-ynyl]thieno[3,2-d]pyrimidin-4-amine;
N-{3-chloro-4-[(3-fluorobenzyl)oxy]phenyl}-6-[3-(isopropylamino)prop-1-ynyl]thieno[3,2-d]pyrimidin-4-amine;
6-[3-(benzylamino)prop-1-ynyl]-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-(3-{methyl[2-(methylsulfonyl)ethyl]amino}prop-1-ynyl)thieno[3,2-d]pyrimidin-4-amine;
3-[4-({3-chloro-4-[(3-fluorobenzyl)oxy]pheny}amino)thieno[3,2-d]pyrimidin-6-yl]prop-2-ynyl methanesulfonate;
3-({3-[4-(}3-chloro-4-[(3-fluorobenzyl)oxy]pheny}amino)thieno[3,2-d]pyrimidin-6-yl]prop-2-ynyl}amino)propanenitrile;
N-{3-chloro-4-[(3-fluorobenzyl)oxy]phenyl}-6-[3-(ethylamino)prop-1-ynyl]thieno[3,2-d]pyrimidin-4-amine;
N-{3-chloro-4-[(3-fluorobenzyl)oxy]phenyl}-6-[3-(propylamino)prop-1-ynyl]thieno[3,2-d]pyrimidin-4-amine;
6-(3-amino-3-methylbut-1-ynyl)-N-{3-chloro-4-[(3-fluorobenzyl)oxy]phenyl}thieno[3,2-d]pyrimidin-4-amine;
N-{3-[4-({3-chloro-4-[(3-fluorobenzyl)oxy]phenyl}amino)thieno[3,2-d]pyrimidin-6-yl]prop-2-ynyl}-2-(1H-indol-3-yl)acetamide;
N-{3-[4-({3-chloro-4-[(3-fluorobenzyl)oxy]phenyl}amino)thieno[3,2-d]pyrimidin-6-yl]prop-2-ynyl}-2-(4-iodophenyl)acetamide;
N-{3-[4-({3-chloro-4-[(3-fluorobenzyl)oxy]phenyl}amino)thieno[3,2-d]pyrimidin-6-yl]prop-2-ynyl}-2-(3,4-dichlorophenyl)acetamide;
N-{3-chloro-4-[(3-fluorobenzyl)oxy]phenyl}-6-[3-(diethylamino)prop-1-ynyl]thieno[3,2-d]pyrimidin-4-amine;
6-(3-aminoprop-1-ynyl)-N-[4-(1-naphthyloxy)phenyl]thieno[3,2-d]pyrimidin-4-amine;
N-{3-chloro-4-[(3-fluorobenzyl)oxy]phenyl}-6-[3-(methylamino)prop-1-ynyl]thieno[3,2-d]pyrimidin-4-amine;
tert-butyl 3-[4-({3-chloro-4-[(3-fluorobenzyl)oxy]phenyl}amino)thieno[3,2-d]pyrimidin-6-yl]prop-2-ynyl(methyl)carbamate hydrochloride;
N-{3-chloro-4-[(3-fluorobenzyl)oxy]phenyl}-6-[3-(dimethylamino)prop-1-ynyl]thieno[2,3-d]pyrimidin-4-amine;
tert-butyl 3-[4-({3-chloro-4-[(3-fluorobenzyl)oxy]phenyl}amino)thieno[2,3-d]pyrimidin-6-yl]prop-2-ynylcarbamate;
N-{3-chloro-4-[(3-fluorobenzyl)oxy]phenyl}-6-[3-(isopropylamino)prop-1-ynyl]thieno[3,2-d]pyrimidin-4-amine;
N-{3-chloro-4-[(3-fluorobenzyl)oxy]phenyl}-6-(3-{methyl[2-(methylsulfonyl)ethyl]amino}prop-1-ynyl)thieno[3,2-d]pyrimidin-4-amine;
N-{3-chloro-4-[(3-fluorobenzyl)oxy]phenyl}-6-[3-(dipropylamino)prop-1-ynyl]thieno[3,2-d]pyrimidin-4-amine hydrochloride;
N-{3-chloro-4-[(3-fluorobenzyl)oxy]phenyl}-6-[3-(diisobutylamino)prop-1-ynyl]thieno[3,2-d]pyrimidin-4-amine;
6-(3-aminoprop-1-ynyl)-N-{3-chloro-4-[(3-fluorobenzyl)oxy]phenyl}thieno[2,3-d]pyrimidin-4-amine;
N-{3-chloro-4-[(3-fluorobenzyl)oxy]phenyl}-6-(pyrimidin-2-ylethynyl)thieno[3,2-d]pyrimidin-4-amine;
6-[3-(benzylamino)prop-1-ynyl]-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-(pyrimidin-2-ylethynyl)thieno[2,3-d]pyrimidin-4-amine;
N-{3-chloro-4-[(3-fluorobenzyl)oxy]phenyl}-6-[3-(methylamino)prop-1-ynyl]thieno[2,3-d]pyrimidin-4-amine;
N-{3-chloro-4-[(3-fluorobenzyl)oxy]phenyl}-6-{3-[(pyridin-4-ylmethyl)amino]prop-1-ynyl}thieno[3,2-d]pyrimidin-4-amine;
N-{3-chloro-4-[(3-fluorobenzyl)oxy]phenyl}-6-(pyridin-2-ylethynyl)thieno[2,3-d]pyrimidin-4-amine;
N-{3-chloro-4-[(3-fluorobenzyl)oxy]phenyl}-6-(pyridin-2-ylethynyl)thieno[3,2-d]pyrimidin-4-amine;
N-{3-[4-({3-chloro-4-[(3-fluorobenzyl)oxy]phenyl}amino)thieno[2,3-d]pyrimidin-6-yl]prop-2-ynyl}-N′-[2-(methylsulfonyl)ethyl]urea;
N-{3-[4-({3-chloro-4-[(3-fluorobenzyl)oxy]phenyl}amino)thieno[2,3-d]pyrimidin-6-yl]prop-2-ynyl}-N′-(2-cyanoethyl)urea;
N′-{3-[4-({3-chloro-4-[(3-fluorobenzyl)oxy]phenyl}amino)thieno[2,3-d]pyrimidin-6-yl]prop-2-ynyl}-N, N-dimethylurea;
N-{3-[4-({3-chloro-4-[(3-fluorobenzyl)oxy]phenyl}amino)thieno[2,3-d]pyrimidin-6-yl]prop-2-ynyl}-N′-[2-(dimethylamino)ethyl]urea;
N-(3-chloro-4-[(3-fluorobenzyl)oxy]phenyl)-6-[3-(diisopentylamino)prop-1-ynyl]thieno[3,2-d]pyrimidin-4-amine hydrochloride;
N-{3-chloro-4-[(3-fluorobenzyl)oxy]phenyl}-6-[3-(1,1-dioxidothiomorpholin-4-yl)prop-1-ynyl]thieno[2,3-d]pyrimidin-4-amine;
N-{3-[4-({3-chloro-4-[(3-fluorobenzyl)oxy]phenyl}amino)thieno[2,3-d]pyrimidin-6-yl]prop-2-ynyl}urea;
N-{3-[4-({3-chloro-4-[(3-fluorobenzyl)oxy]phenyl}amino)thieno[2,3-d]pyrimidin-6-yl]prop-2-ynyl}acetamide;
N-{3-chloro-4-[(3-fluorobenzyl)oxy]phenyl}-6-(1,3-thiazol-2-ylethynyl)thieno[3,2-d]pyrimidin-4-amine;
N-{3-chloro-4-[(3-fluorobenzyl)oxy]phenyl}-6-(3-piperidin-1-ylprop-1-ynyl)thieno[3,2-d]pyrimidin-4-amine;
5-{[4-({3-chloro-4-[(3-fluorobenzyl)oxy]phenyl}amino)thieno[3,2-d]pyrimidin-6-yl]ethynyl)-2-furaldehyde;
N-{3-[4-({3-chloro-4-[(3-fluorobenzyl)oxy]phenyl}amino)thieno[2,3-d]pyrimidin-6-yl]prop-2-ynyl}-2-pyridin-4-ylacetamide;
N-{3-[4-({3-chloro-4-[(3-fluorobenzyl)oxy]phenyl}amino)thieno[2,3-d]pyrimidin-6-yl]prop-2-ynyl}-2-pyridin-2-ylacetamide;
N-{3-[4-({3-chloro-4-[(3-fluorobenzyl)oxy]phenyl}amino)thieno[2,3-d]pyrimidin-6-yl]prop-2-ynyl}-2-(1-methyl-1H-imidazol-4-yl)acetamide;
N-{3-[4-({3-chloro-4-[(3-fluorobenzyl)oxy]phenyl}amino)thieno[2,3-d]pyrimidin-6-yl]prop-2-ynyl}-2-thien-3-ylacetamide;
N-{3-[4-({3-chloro-4-[(3-fluorobenzyl)oxy]phenyl}amino)thieno[2,3-d]pyrimidin-6-yl]prop-2-ynyl}cinnoline-4-carboxamide;
3-[4-(13-chloro-4-[(3-fluorobenzyl)oxy]phenyl}amino)thieno[3,2-d]pyrimidin-6-yl]prop-2-ynyl methanesulfonate;
N-{3-chloro-4-[(3-fluorobenzyl)oxy]phenyl}-6-(1,3-thiazol-2-ylethynyl)thieno[2,3-d]pyrimidin-4-amine;
6-(3-amino-3-methylbut-1-ynyl)-N-{3-chloro-4-[(3-fluorobenzyl)oxy]phenyl}thieno[3,2-d]pyrimidin-4-amine;
N-{3-chloro-4-[(3-fluorobenzyl)oxy]phenyl}-6-[3-(ethylamino)prop-1-ynyl]thieno[3,2-d]pyrimidin-4-amine;
3-(}3-[4-(13-chloro-4-[(3-fluorobenzyl)oxy]phenyl}amino)thieno[3,2-d]pyrimidin-6-yl]prop-2-ynyl}amino)propanenitrile;
N-{3-chloro-4-[(3-fluorobenzyl)oxy]phenyl)-6-{3-[(2-methoxyethyl)amino]prop-1-ynyl}thieno[3,2-d]pyrimidin-4-amine;
N-{3-chloro-4-[(3-fluorobenzyl)oxy]phenyl}-6-[3-(propylamino)prop-1-ynyl]thieno[3,2-d]pyrimidin-4-amine;
N-{3-chloro-4-[(3-fluorobenzyl)oxy]phenyl}-6-{[5-({[2-(methylsulfonyl)ethyl]aminolmethyl)-2-furyl]ethynylthieno[3,2-d]pyrimidin-4-amine;
N-{3-chloro-4-[(3-fluorobenzyl)oxy]phenyl}-6-(3-{[2-(1H-imidazol-4-yl)ethyl]amino}prop-1-ynyl)thieno[3,2-d]pyrimidin-4-amine;
4-[4-({3-chloro-4-[(3-fluorobenzyl)oxy]phenyl}amino)thieno[3,2-d]pyrimidin-6-yl]-2-methylbut-3-yn-2-ol;
N-(3-chloro-4-[(3-fluorobenzyl)oxy]phenyl}-6-(1H-imidazol-4-ylethynyl)thieno[3,2-d]pyrimidin-4-amine;
4-[4-((3-chloro-4-[(3-fluorobenzyl)oxy]phenylamino)thieno[3,2-d]pyrimidin-6-yl]but-3-yn-1-ol;
N-{3-chloro-4-[(3-fluorobenzyl)oxy]phenyl}-6-(phenylethynyl)thieno[3,2-d]pyrimidin-4-amine;
N-{3-chloro-4-[(3-fluorobenzyl)oxy]phenyl}-6-{[6-({[2-(methylsulfonyl)ethyl]aminomethyl)pyridin-2-yl]ethynylthieno[2,3-d]pyrimidin-4-amine hydrochloride; and
6-{[4-({3-chloro-4-[(3-fluorobenzyl)oxy]phenyl}amino)thieno[2,3-d]pyrimidin-6-yl]ethynyl}pyridine-2-carbaldehyde.

20. A compound selected from the group consisting of:
(R,S)-6-(3-aminobut-1-ynyl)-N-{3-chloro-4-[(3-fluorobenzyl)oxy]phenyl}thieno[3,2-d]pyrimidin-4-amine;
(R)-6-(3-aminobut-1-ynyl)-N-{3-chloro-4-[(3-fluorobenzyl)oxy]phenyl}thieno[3,2-d]pyrimidin-4-amine;
(S)-6-(3-aminobut-1-ynyl)-N-{3-chloro-4-[(3-fluorobenzyl)oxy]phenyl}thieno[3,2-d]pyrimidin-4-amine;
(R,S)-6-(3-aminopent-1-ynyl)-N-{3-chloro-4-[(3-fluorobenzyl)oxy]phenyl}thieno[3,2-d]pyrimidin-4-amine;
(R,S)-6-(3-aminobut-1-ynyl)-N-[3-chloro-4-(1-naphthyloxy)phenyl]thieno[3,2-d]pyrimidin-4-amine;
(R,S)-6-(3-aminobut-1-ynyl)-N-(2-benzyl-1H-benzimidazol-5-yl)thieno[3,2-d]pyrimidin-4-amine;
(R,S)-6-(3-aminobut-1-ynyl)-N-[1-(pyridin-3-ylmethyl)-1H-indol-5-yl]thieno[3,2-d]pyrimidin-4-amine;
(R,S)—N⁴-[6-(3-aminobut-1-ynyl)thieno[3,2-d]pyrimidin-4-yl]-2-chloro-N¹-(3-fluorobenzyl)benzene-1,4-diamine;
(R,S)-6-(3-aminobut-1-ynyl)-N-[1-(3-fluorobenzyl)-1H-indazol-5-yl]thieno[3,2-d]pyrimidin-4-amine;
(R,S)-6-(3-aminobut-1-ynyl)-N-{3-fluoro-4-[(3-fluorobenzyl)oxy]phenyl}thieno[3,2-d]pyrimidin-4-amine;
(R,S)-6-(3-aminobut-1-ynyl)-N-(4-benzylphenyl)thieno[3,2-d]pyrimidin-4-amine;
(R,S)-6-(3-aminobut-1-ynyl)-N-[1-(2-fluorobenzyl)-1H-indazol-5-yl]thieno[3,2-d]pyrimidin-4-amine;
(R,S)-6-(3-aminobut-1-ynyl)-N-[2-(2-fluorobenzyl)-1H-benzimidazol-5-yl]thieno[3,2-d]pyrimidin-4-amine;
(R,S)-6-(3-aminobut-1-ynyl)-N-[1-(2,5-difluorobenzyl)-1H-indol-5-yl]thieno[3,2-d]pyrimidin-4-amine;
(R,S)-6-(3-aminobut-1-ynyl)-N-(1-benzyl-1H-indol-5-yl)thieno[3,2-d]pyrimidin-4-amine;
(R,S)-6-(3-aminobut-1-ynyl)-N-(1-benzyl-1H-indazol-5-yl)thieno[3,2-d]pyrimidin-4-amine;
(R,S)-6-(3-aminobut-1-ynyl)-N-[2-(3-fluorobenzyl)-1H-benzimidazol-5-yl]thieno[3,2-d]pyrimidin-4-amine;
(2R,S)—N-{3-chloro-4-[(3-fluorobenzyl)oxy]phenyl)-6-{3-[(2-methoxyethyl)amino]but-1-ynyl}thieno[3,2-d]pyrimidin-4-amine;
(2R)-2-amino-4-[4-({3-chloro-4-[(3-fluorobenzyl)oxy]phenyl}amino)thieno[3,2-d]pyrimidin-6-yl]but-3-yn-1-ol; and
(2S)-2-amino-4-[4-({3-chloro-4-[(3-fluorobenzyl)oxy]phenyl}amino)thieno[3,2-d]pyrimidin-6-yl]but-3-yn-1-ol.

21. A method for treating a disorder in a mammal characterized by aberrant activity of at least one protein tyrosine kinase, said method comprising administering to said mammal a therapeutically effective amount of a compound according to claim 1, or a salt thereof.

22. A method according to claim 21, wherein said protein tyrosine kinase is EGFR, c-Erb-B2,or c-Erb-B4.

23. A method according to claim 21, wherein said method further comprises administering at least one additional anti-neoplastic agent.

24. A method according to claim 23, wherein said additional anti-neoplastic agent is selected from the group consisting of paclitaxel, docetaxel, vinblastine, vincristine, vindesine, vinorelbine, 5-fluorouracil, fluorodeoxyuridine, allopurinol, fludurabine, methotrexate, cladrabine, cytarabine, mercaptopurine, thioguanine, 9-amino camptothecin, irinotecan, CPT-11, 7-(4-methylpiperazino-methylene)-10, 11-ethylenedioxy-20-camptothecin, melphalan, chlorambucil, cyclophosphamide, mechlorethamine, hexamethylmelamine, busulfan, carmustine, lomustine, dacarbazine, doxorubicin, daunomycin, epirubicin, idarubicin, mitomycin-C, dacttinomycin, mithramycin, cisplatin, carboplatin, oxaliplatin, tamoxifen, toremifene, raloxifene, droloxifene, iodoxyfene, megestrol acetate, anastrozole, letrazole, vorazole, exemestane, flutamide, nilutamide, bicalutamide, cyproterone acetate, goserelin acetate, uprolide, finasteride, marimastat, antiprogestogens, urokinase plasminogen activator receptor function inhibitors, celecoxib, VEGFR inhibitors, TIE-2inhibitors, growth factor function inhibitors, and inhibitors of CDK2and CDK4.

25. A method for treating a disorder in a mammal characterized by aberrant activity of at least one ErbB family protein tyrosine kinase, said method comprising administering to said mammal a therapeutically effective amount of a compound according to claim 19, or a salt thereof.

26. A method according to claim 25, wherein said protein tyrosine kinase is EGFR, c-Erb-B2,or c-Erb-B4.

27. A method according to claim 25, wherein said method further comprises administering at least one additional anti-neoplastic agent.

28. A method according to claim 27, wherein said additional neo-plastic agent is selected from the group consisting of paclitaxel, docetaxel, vinblastine, vincristine, vindesine, vinorelbine, 5-fluorouracil, fluorodeoxyuridine, allopurinol, fludurabine, methotrexate, cladrabine, cytarabine, mercaptopurine, thioguanine, 9-amino camptothecin, irinotecan, CPT-11, 7-(4-methylpiperazino-methylene)-10,11-ethylenedioxy-20-camptothecin, emelphalan, chlorambucil, cyclophosphamide, mechlorethamine, hexamethylmelamine, busulfan, carmustine, lomustine, dacarbazine, doxorubicin, daunomycin, epirubicin, idarubicin, mitomycin-C, dacttinomycin, mithramycin, cisplatin, carboplatin, oxaliplatin, tamoxifen, toremifene, raloxifene, droloxifene, iodoxyfene, megestrol acetate, anastrozole, letrazole, vorazole, exemestane, flutamide, nilutamide, bicalutamide, cyproterone acetate, goserelin acetate, uprolide, finasteride, marimastat, antiprogestogens, urokinase plasminogen activator receptor function inhibitors, celecoxib, VEGFR inhibitors, TIE-2inhibitors, growth factor function inhibitors, and inhibitors of CDK2and CDK4.

29-32. (Cancelled):

33. A pharmaceutical composition for the treatment of cancer and malignant tumors in an animal, said composition comprising a compound according to claim 1, or a salt thereof.

34. A pharmaceutical composition for the treatment of cancer and malignant tumors in an animal, said composition comprising a compound according to claim 19, or a salt thereof.

35. A method for the treatment of cancer and malignant tumors in an animal, said method comprising administering a therapeutically effective amount of a compound according to claim 1, or a salt thereof.

36. A method for the treatment of cancer and malignant tumors in an animal, said method comprising administering a therapeutically effective amount of a compound according to claim 19, or a salt thereof.

37-38. (Cancelled)

39. A method for treating a disorder in a mammal characterized by aberrant activity of at least one ErbB family protein tyrosine kinase, said method comprising administering to said mammal a therapeutically effective amount of a compound according to claim 20, or a salt thereof.

40. A method according to claim 39, wherein said protein tyrosine kinase is EGFR, c-Erb-B2,or c-Erb-B4.

41. A method according to claim 39, wherein said method further comprises administering at least one additional anti-neoplastic agent.

42. A method according to claim 41, wherein said additional neo-plastic agent is selected from the group consisting of paclitaxel, docetaxel, vinblastine, vincristine, vindesine, vinorelbine, 5-fluorouracil, fluorodeoxyuridine, allopurinol, fludurabine, methotrexate, cladrabine, cytarabine, mercaptopurine, thioguanine, 9-amino camptothecin, irinotecan, CPT-11, 7-(4-methylpiperazino-methylene)-10,11-ethylenedioxy-20-camptothecin, melphalan, chlorambucil, cyclophosphamide, mechlorethamine, hexamethylmelamine, busulfan, carmustine, lomustine, dacarbazine, doxorubicin, daunomycin, epirubicin, idarubicin, mitomycin-C, dacttinomycin, mithramycin, cisplatin, carboplatin, oxaliplatin, tamoxifen, toremifene, raloxifene, droloxifene, iodoxyfene, megestrol acetate, anastrozole, letrazole, vorazole, exemestane, flutamide, nilutamide, bicalutamide, cyproterone acetate, goserelin acetate, uprolide, finasteride, marimastat, antiprogestogens, urokinase plasminogen activator receptor function inhibitors, celecoxib, VEGFR inhibitors, TIE-2inhibitors, growth factor function inhibitors, and inhibitors of CDK2and CDK4.

43. A pharmaceutical composition for the treatment of cancer and malignant tumors in an animal, said composition comprising a compound according to claim 20, or a salt thereof.

44. A method for the treatment of cancer and malignant tumors in an animal, said method comprising administering a therapeutically effective amount of a compound according to claim 20, or a salt thereof.
